SEQUENCE LISTING (89 sequences)

SEQ ID NO: 1 - DNA, 66 bp, mouse mammary tumour virus
tacgcgtcggctggctgcaagaatttcttctggaagactttcactagttgcgcgtataccgcgtcc

SEQ ID NO: 2 - DNA, 2064 bp, mouse mammary tumour virus
atgccgaaacaccaatctgggtccccgatcggttcatccgaccttttactgagcggaaagaagcaacgcccacacctggcactgcggagaaaacgccgccgcgagatgagaaagatcaacagaaaagtccggaggatgaatctagcccccatcaaagagaagacggcttggcaacatctgcaggcgttaatcttcgaagcggaggaggttcttaaaacctcacaaactccccaaacctctttgactttatttcttgctttgttgtctgtcctcggccccccgcctgtgaccggggaaagttattgggcttacctacctaaaccacctattctccatcccgtgggatggggaaatacagaccccattagagttctgaccaatcaaaccatatatttgggtgggtcgcctgactttcacgggtttagaaacatgtctggcaatgtacattttgaggggaagtctgatacgctccccatttgcttttccttctccttttctacccccacaggctgctttcaagtagataagcaagtatttctttctgatacacccacggttgataataataaacctgggggaaagggtgataaaaggcgtatgtgggaactctggttgactactttggggaactcaggggccaatacaaaactggtccctataaagaagaagttgccccccaaatatcctcactgccagatcgcctttaagaaggacgccttctgggagggagacgagtctgctcctccacggtggttgccttgcgccttccctgaccagggggtgagtttttctccaaaaggggcccttgggttactttgggatttctcccttccctcgcctagtgtagatcagtcagatcagattaaaagcaaaaaggatctatttggaaattatactccccctgtcaataaagaggttcatcgatggtatgaagcaggatgggtagaacctacatggttctgggaaaattctcctaaggatcccaatgatagagattttactgctctagttccccatacagaattgtttcgcttagttgcagcctcaagatatcttattctcaaaaggccaggatttcaagaacatgacatgattcctacatctgcctgtgttacttaccctcatgccatattattaggattacctcagctaatagatatagagaaaagaggatctacttttcatatttcctgttcttcttgtagattgactaattgtttagattcttctgcctacgactatgcagcgatcatagtcaagaggccgccatacgtgctgctacctgtagatattggtgatgaaccatggtttgatgattctgccattcaaacctttaggtatgccacagatttaattcgagccaagcgattcgtcgctgccattattctgggcatatctgctttaattgctattatcacttcctttgctgtagctactactgctttagttaaggagatgcaaactgctacgtttgttaataatcttcataggaatgttacattagctttatctgaacaaagaataatagatttaaaattagaagctagacttaatgctttagaagaagtagttttagagttgggacaagatgtggcaaacttaaagaccagaatgtccaccaggtgtcatgcaaattatgattttatctgcgttacacctttaccatataatgcttctgagagctgggaaagaaccaaagctcatttattgggcatttggaatgacaatgagatttcatataacatacaagaattaaccaacctgattagtgatatgagcaaacaacatattgacacagtggacctcagtggcttggctcagtcctttgccaatggagtaaaggctttaaatccattagattggacacaatatttcatttttataggtgttggagccctgcttttagtcatagtgcttatgattttccccattgttttccagtgccttgcgaagagccttgaccaagtgcagtcagatcttaacgtgcttcttttaaaaaagaaaaaagggggaaatgccgcgcctgcagcagaaatggttgaactcccgagagtgtcctacacc

SEQ ID NO: 3 - DNA, 15 bp, mouse mammary tumour virus
tttcacgggtttaga

SEQ ID NO: 4 - DNA, 21 bp, mouse mammary tumour virus
gactttcacgggtttagaaac

SEQ ID NO: 5 - DNA, 27 bp, mouse mammary tumour virus
cctgactttcacgggtttagaaacatg

SEQ ID NO: 6 - DNA, 36 bp, mouse mammary tumour virus
tcgcctgactttcacgggtttagaaacatgtctggc

SEQ ID NO: 7 - DNA, 39 bp, mouse mammary tumour virus
gggtcgcctgactttcacgggtttagaaacatgtctggc

SEQ ID NO: 8 - DNA, 42 bp, mouse mammary tumour virus
ggtgggtcgcctgactttcacgggtttagaaacatgtctggc

SEQ ID NO: 9 - protein, 21 aa, mouse mammary tumour virus
YASAGCKNFFWKTFTSCYTAS

SEQ ID NO: 10 - protein, 688 aa, mouse mammary tumour virus
MPKHQSGSPIGSSDLLLSGKKQRPHLALRRKRRREMRKINRKVRRMNLAPIKEKTAWQHLQALIFEAEEVLKTSQTPQTSLTLFLALLSVLGPPPVTGESYWAYLPKPPILHPVGWGNTDPIRVLTNQTIYLGGSPDFHGFRNMSGNVHFEGKSDTLPICFSFSFSTPTGCFQVDKQVFLSDTPTVDNNKPGGKGDKRRMWELWLTTLGNSGANTKLVPIKKKLPPKYPHCQIAFKKDAFWEGDESAPPRWLPCAFPDQGVSFSPKGALGLLWDFSLPSPSVDQSDQIKSKKDLFGNYTPPVNKEVHRWYEAGWVEPTWFWENSPKDPNDRDFTALVPHTELFRLVAASRYLILKRPGFQEHDMIPTSACVTYPHAILLGLPQLIDIEKRGSTFHISCSSCRLTNCLDSSAYDYAAIIVKRPPYVLLPVDIGDEPWFDDSAIQTFRYATDLIRAKRFVAAIILGISALIAIITSFAVATTALVKEMQTATFVNNLHRNVTLALSEQRIIDLKLEARLNALEEVVLELGQDVANLKTRMSTRCHANYDFICVTPLPYNASESWERTKAHLLGIWNDNEISYNIQELTNLISDMSKQHIDTVDLSGLAQSFANGVKALNPLDWTQYFIFIGVGALLLVIVLMIFPIVFQCLAKSLDQVQSDLNVLLLKKKKGGNAAPAAEMVELPRVSYT

SEQ ID NO: 11 - protein, 5 aa, mouse mammary tumour virus
FHGFR

SEQ ID NO: 12 - protein, 7 aa, mouse mammary tumour virus
DFHGFRN

SEQ ID NO: 13 - protein, 9 aa, mouse mammary tumour virus
PDFHGFRNM

SEQ ID NO: 14 - protein, 11 aa, mouse mammary tumour virus
SPDFHGFRNMS

SEQ ID NO: 15 - protein, 12 aa, mouse mammary tumour virus
SPDFHGFRNMSG

SEQ ID NO: 16 - protein, 14 aa, mouse mammary tumour virus
GGSPDFHGFRNMSG

SEQ ID NO: 17 - DNA, 57 bp, mouse mammary tumour virus
caaaccatatatttgggtgggtcgcctgactttcacgggtttagaaacatgtctggc

SEQ ID NO: 18 - DNA, 117 bp, mouse mammary tumour virus
ggtgggtcgcctgactttcacgggtttagaaacatgtctggcaatgtacattttgaggggaagtctgatacgctccccatttgcttttccttctccttttctacccccacaggctgc

SEQ ID NO: 19 - protein, 8 aa, Artificial
  Artificially ligated
DYKDDDDK

SEQ ID NO: 20 - protein, 8 aa, Artificial
  Artificially ligated
DYKDEDDK

SEQ ID NO: 21 - protein, 9 aa, Artificial
  Artificial
AWRHPQFGG

SEQ ID NO: 22 - protein, 13 aa, Influenza virus
YPYDVPDYAIEGR

SEQ ID NO: 23 - protein, 3 aa, Artificial
  Artificially ligated
EEF

SEQ ID NO: 24 - protein, 6 aa, Artificial
  Artificially ligated
HHHHHH

SEQ ID NO: 25 - protein, 11 aa, Homo sapiens
EQKLLSEEDLN

SEQ ID NO: 26 - DNA, 117 bp, mouse mammary tumour virus; identical to SEQ ID NO: 18

SEQ ID NO: 27 - DNA, 27 bp, mouse mammary tumour virus
ataaagaagaagttgccccccaaatat

SEQ ID NO: 28 - DNA, 33 bp, mouse mammary tumour virus
cctataaagaagaagttgccccccaaatatcct

SEQ ID NO: 29 - DNA, 39 bp, mouse mammary tumour virus
gtccctataaagaagaagttgccccccaaatatcctcac

SEQ ID NO: 30 - DNA, 45 bp, mouse mammary tumour virus
ctggtccctataaagaagaagttgccccccaaatatcctcactgc

SEQ ID NO: 31 - DNA, 51 bp, mouse mammary tumour virus
aaactggtccctataaagaagaagttgccccccaaatatcctcactgccag

SEQ ID NO: 32 - DNA, 57 bp, mouse mammary tumour virus
cctgggggaaagggtgataaaaggcgtatgtgggaactctggttgactactttgggg

SEQ ID NO: 33 - protein, 9 aa, mouse mammary tumour virus
IKKKLPPKY

SEQ ID NO: 34 - protein, 11 aa, mouse mammary tumour virus
PIKKKLPPKYP

SEQ ID NO: 35 - protein, 12 aa, mouse mammary tumour virus
PIKKKLPPKYPH

SEQ ID NO: 36 - protein, 15 aa, mouse mammary tumour virus
LVPIKKKLPPKYPHC

SEQ ID NO: 37 - protein, 17 aa, mouse mammary tumour virus
KLVPIKKKLPPKYPHCQ

SEQ ID NO: 38 - protein, 19 aa, mouse mammary tumour virus
TKLVPIKKKLPPKYPHCQI

SEQ ID NO: 39 - DNA, 1995 bp, mouse mammary tumour virus
atggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtgggactcctctctcccctacccaacttccaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagcca

SEQ ID NO: 40 - protein, 665 aa, mouse mammary tumour virus
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 41 - DNA, 2019 bp, mouse mammary tumour virus
atggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttacgcgtcggctggctgcaagaatttcttctggaagactttcactagttgcgcgtataccgcgtccgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtgggactcctctctcccctacccaacttccaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagcca

SEQ ID NO: 42 - protein, 672 aa, mouse mammary tumour virus
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPYASAGCKNFFWKTFTSCYTASEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 43 - DNA, 235 bp, mouse mammary tumour virus
gcagcagaaatggttgaactcccgagagtgtcctacacctaggggagaagcagccgtcatagtgcttatgattttccccattgttttccagtgccttgcgaagagccttgaccaagtgcagtcagatcttaacgtgcttcttttaaaaaagaaaaaagggggaaatgccgcgcctttaaatccattagattggacacaatatttcatttttataggtgttggagccctgctttta

SEQ ID NO: 44 - protein, 73 aa, mouse mammary tumour virus
LNPLDWTQYFIFIGVGALLLVIVLMIFPIVFQCLAKSLDQAAEMVELPRVSYTVQSDLNVLLLKKKKGGNAAP

SEQ ID NO: 45 - protein, 68 aa, mouse mammary tumour virus
LKPIEYEPLIVLLMILLFGPCILNRLVQFVKDRISVVQALVLTQQYHQEGLFNRSPWFTTLISTIMGP

SEQ ID NO: 46 - DNA, 207 bp, mouse mammary tumour virus
gagggactgtttaacagatccccttggtttaccaccttgatatctaccattatgggacccctcattgtactcctaatgattttgctcttcggaccctgcattcttaatcgattagtccaactgaagcctatagagtacgagccatagtttgttaaagacaggatatcagtggtccaggctctagttttgactcaacaatatcaccag

SEQ ID NO: 47 - DNA, 21 bp, mouse mammary tumour virus
cttgtgtttttccacaggatg

SEQ ID NO: 48 - DNA, 33 bp, mouse mammary tumour virus
tgcgaattcctatcgcttggctcgaattaaatc

SEQ ID NO: 49 - DNA, 42 bp, mouse mammary tumour virus
tgttgctcagggggcagcagcccaggctgttccagagactgc

SEQ ID NO: 50 - protein, 14 aa, mouse mammary tumour virus
CCSGGSSPGCSRDC

SEQ ID NO: 51 - protein, 14 aa, mouse mammary tumour virus
LGGSPDFHGFRNMS

SEQ ID NO: 52 - protein, 9 aa, mouse mammary tumour virus
YLGGSPDFH

SEQ ID NO: 53 - protein, 19 aa, mouse mammary tumour virus
QTIYLGGSPDFHGFRNMSG

SEQ ID NO: 54 - protein, 39 aa, mouse mammary tumour virus
GGSPDFHGFRNMSGNVHFEGKSDTLPICFSFSFSTPTGC

SEQ ID NO: 55 - protein, 44 aa, mouse mammary tumour virus
QTIYLGGSPDFHGFRNMSGNVHFEGKSDTLPICFSFSFSTPTGC

SEQ ID NO: 56 - DNA, 21 bp, mouse mammary tumour virus
aagaagaagttgccccccaaa

SEQ ID NO: 57 - DNA, 45 bp, mouse mammary tumour virus
acaaaactggtccctataaagaagaagttgccccccaaatatcct

SEQ ID NO: 58 - DNA, 117 bp, mouse mammary tumour virus
cctgggggaaagggtgataaaaggcgtatgtgggaactctggttgactactttggggaactcaggggccaatacaaaactggtccctataaagaagaagttgccccccaaatatcct

SEQ ID NO: 59 - DNA, 102 bp, mouse mammary tumour virus
aagggtgataaaaggcgtatgtgggaactctggttgactactttggggaactcaggggccaatacaaaactggtccctataaagaagaagttgccccccaaa

SEQ ID NO: 60 - DNA, 66 bp, mouse mammary tumour virus
cctgggggaaagggtgataaaaggcgtatgtgggaactctggttgactactttggggaactcaggg

SEQ ID NO: 61 - DNA, 192 bp, mouse mammary tumour virus
aaacctgggggaaagggtgataaaaggcgtatgtgggaactctggttgactactttggggaactcaggggccaatacaaaactggtccctataaagaagaagttgccccccaaatatcctcactgccagatcgcctttaagaaggacgccttctgggagggagacgagtctgctcctccacggtggttgcct

SEQ ID NO: 62 - protein, 7 aa, mouse mammary tumour virus
KKKLPPK

SEQ ID NO: 63 - protein, 15 aa, mouse mammary tumour virus
TKLVPIKKKLPPKYP

SEQ ID NO: 64 - protein, 39 aa, mouse mammary tumour virus
PGGKGDKRRMWELWLTTLGNSGANTKLVPIKKKLPPKYP

SEQ ID NO: 65 - protein, 34 aa, mouse mammary tumour virus
KGDKRRMWELWLTTLGNSGANTKLVPIKKKLPPK

SEQ ID NO: 66 - protein, 6 aa, mouse mammary tumour virus
KGDKRR

SEQ ID NO: 67 - protein, 19 aa, mouse mammary tumour virus
PGGKGDKRRMWELWLTTLG

SEQ ID NO: 68 - protein, 22 aa, mouse mammary tumour virus
PGGKGDKRRMWELWLTTLGNSG

SEQ ID NO: 69 - protein, 64 aa, mouse mammary tumour virus
KPGGKGDKRRMWELWLTTLGNSGANTKLVPIKKKLPPKYPHCQIAFKKDAFWEGDESAPPRWLP

SEQ ID NO: 70 - DNA, 42 bp, mouse mammary tumour virus; identical to SEQ ID NO: 49

SEQ ID NO: 71 - DNA, 15 bp, mouse mammary tumour virus
tgttccagagactgc

SEQ ID NO: 72 - DNA, 25 bp, mouse mammary tumour virus
ctgttccagagactgcgaagaacct

SEQ ID NO: 73 - DNA, 132 bp, mouse mammary tumour virus
ccatcttattgggggctagaatatcaatcccctttttcttctcccccggggcccccttgttgctcagggggcagcagcccaggctgttccagagactgcgaagaacctttaacctccctcacccctcggtgc

SEQ ID NO: 74 - DNA, 117 bp, mouse mammary tumour virus
ctagaatatcaatcccctttttcttctcccccggggcccccttgttgctcagggggcagcagcccaggctgttccagagactgcgaagaacctttaacctccctcacccctcggtgc

SEQ ID NO: 75 - DNA, 153 bp, mouse mammary tumour virus
tgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgc

SEQ ID NO: 76 - protein, 14 aa, mouse mammary tumour virus
CCSGGSSPGCSRDC

SEQ ID NO: 77 - protein, 5 aa, mouse mammary tumour virus
CSRDC

SEQ ID NO: 78 - protein, 43 aa, mouse mammary tumour virus
CMLAHHGPSYWGLEYQSPFSSPPGPPCCSGGSSPGCSRDCEEP

SEQ ID NO: 79 - protein, 44 aa, mouse mammary tumour virus
PSYWGLEYQSPFSSPPGPPCCSGGSSPGCSRDCEEPLTSLTPRC

SEQ ID NO: 80 - protein, 39 aa, mouse mammary tumour virus
LEYQSPFSSPPGPPCCSGGSSPGCSRDCEEPLTSLTPRC

SEQ ID NO: 81 - protein, 51 aa, mouse mammary tumour virus
CMLAHHGPSYWGLEYQSPFSSPPGPPCCSGGSSPGCSRDCEEPLTSLTPRC

SEQ ID NO: 82 - DNA, 18 bp, mouse mammary tumour virus
aagggtgataaaaggcgt

SEQ ID NO: 83 - DNA, 30 bp, Artificial
  Artificially ligated
tacgcgtccgaagaacctttaacctccctc

SEQ ID NO: 84 - DNA, 20 bp, mouse mammary tumour virus
agggggccccgggggagaag

SEQ ID NO: 85 - DNA, 57 bp, Artificial
  Artificially ligated
cgcgtcggctggctgcaagaatttcttctggaagactttcactagttgcgcgtatac

SEQ ID NO: 86 - DNA, 57 bp, Artificial
  Artificially ligated
cgcggtatacgcgcaactagtgaaagtcttccagaagaaattcttgcagccagccga

SEQ ID NO: 87 - protein, 686 aa, homo sapiens
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGGGGAGCKNFFWKTFTSCSGGGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 88 - protein, 679 aa, mouse mammary tumour virus
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGAGCKNFFWKTFTSCPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 89 - protein, 688 aa, mouse mammary tumour virus
MARSTLSKPLKNKVNPRGPLIPLILLMLRGVSTSGGGGAGCKNFFWKTFTSCSGGGASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

(19) United States
(12) Patent Application Publication
(10) Pub. No.: US 2007/0003522 A1
(43) Pub. Date: Jan. 4, 2007

(54) Methods and compositions for improved retroviral gene and drug delivery

(76) Inventor: Lorraine M. Albritton, Memphis, TN (US)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/175,401

(22) Filed: Jul. 7, 2005

Related U.S. Application Data
(60) Provisional application No. 60/586,025, filed on Jul. 8, 2004.

(51) Int. Cl. A61K 48/00; C12N 15/861

(52) U.S. Cl. 424/93.2; 435/456; 977/802; 977/906

(57) ABSTRACT

The present invention provides recombinant viral particles for gene therapy and liposome compositions for drug delivery comprising an env protein of MMTV. The invention also provides retroviral or lentiviral env proteins comprising a mutation in a receptor-binding motif. The invention also provides nucleic acids, proteins, and compositions comprising the recombinant viral particles, nucleic acids, and proteins. The invention also provides methods for enhancing delivery of a gene or compound of interest to a target cell, and methods for targeting a compound of interest to an acidified compartment of a cell.

### CROSS REFERENCE TO RELATED APPLICATION

This application claims priority of U.S. Provisional Application Ser. No. 60/586,025, filed Jul. 8, 2004. This application is hereby incorporated in its entirety by reference herein.

### STATEMENT REGARDING FEDERALLY SPONSORED RESEARCH OR DEVELOPMENT

The invention described herein was supported in part by a grant from The National Institutes of Health (Grant No. R01 CA 81171 and R01 AI 33410). The U.S. Government may have certain rights in this invention

### FIELD OF THE INVENTION

The present invention provides recombinant viral particles for gene therapy and liposome compositions for drug delivery comprising a receptor-binding sequence of an envelope (env) protein, mutants thereof, nucleic acids encoding same, proteins and compositions comprising same. The invention also provides methods for enhancing delivery of a nucleic acid or compound of interest to a target cell, and methods for targeting same.

### BACKGROUND OF THE INVENTION

Recombinant viral particles show great promise for many therapeutic applications. Many applications in gene and drug therapy require targeting, or preferentially directing, a viral particle or drug delivery vehicle to a subset of cells in the patient, known as “target cells.” Targeting a viral particle to a specific cell type enhances efficacy of gene and drug therapy by increasing the number of target cells infected or receiving the drug, and improves safety by decreasing the systemic dose required to infect or deliver drug to a given number of target cells and by protecting non-target cells and germline cells from introduction of therapeutic genes and drugs. Targeting is achieved by engineering a viral particle or drug delivery vehicle to utilize a molecule expressed predominantly or exclusively on the target cell.

Retroviruses and lentiviruses are useful agents for gene therapy. These viruses integrate a copy of their genome into the host cell, allowing stable expression of genes contained therein and enhancing effectiveness in gene therapy applications. One class of potentially useful retroviruses is Murine Leukemia Viruses (MLV), which belong to the gamma retrovirus subfamily of retroviruses. Another potentially useful retrovirus is MMTV, which belongs to the beta retrovirus family of retroviruses.

Lentiviruses have the additional advantage of infecting non-dividing as well as dividing cells. Additionally, lentiviruses may not insert their genes upstream of a cellular proto-oncogene, limiting their potential to cause cancer, a complication to date with gene therapy for severe combined immunodeficiency syndrome (Check E et al, Nature 419, 545-546; 2002). However, lentiviruses known to date only infect those that express the CD4 molecule, limiting their utility. Therefore it is important for a variety of clinical applications to design a retroviral or lentiviral delivery vehicle without these limitations.

Drug delivery vehicles are useful agents for delivery of pharmacological therapeutics. Drug delivery vehicles contain high concentrations of drugs or proteins or other pharmacological agent. They fuse with cells, dispensing their contents into the cell and enhancing effectiveness in drug delivery applications. One class of potentially useful drug delivery vehicles is liposomes. However, liposomes to date fuse with any cell that they encounter regardless of whether delivery to that cell would give therapeutic benefit, limiting their effectiveness and utility. Therefore it is important for a variety of clinical applications to design a liposome drug delivery vehicle that gives greater fusion with a specific target cell than to other cells.

Mouse mammary tumor virus (MMTV) is a retrovirus that infects cells expressing mouse transferrin receptor 1 (TfR1). The MMTV env protein directs high affinity/strong retrovirus particle binding to TfR1 and the retrovirus particle-TfR1 complex internalizes to low pH compartments of the host cell where the acidic conditions induce the MMTV env protein to fuse the retrovirus membrane with the cell membrane, delivering a copy of the virus genome into the cell.

Moloney murine leukemia virus (MoMLV) and Friend-MLV are retroviruses that exclusively infects cells expressing mouse cationic amino acid transporter type 1 (ATRC1, also referred to as MCAT-1). The MoMLV envelope (env) protein or/and the Friend-MLV env protein direct high affinity retrovirus particle binding to ATRC1 and the retrovirus particle-ATRC1 complex internalizes to intracellular compartments of the host cell where the interactions of env protein with ATRC1 induce the MoMLV and the Friend-MLV env protein to fuse the retrovirus membrane with the cell membrane, delivering a copy of the virus genome into the cell.

The present invention delineates the receptor binding domain (RBD) of MMTV env protein, the receptor-binding motif (RBM), and the heparin sulfate-binding motif (HBM) of MMTV env protein, and the receptor-binding motif of MoMLV env protein, and describes modifications to the env, RBD, RBM, and HBM of MMTV env, MoMLV env, lentiviral env and other retroviral env that have utility in gene therapy and drug delivery, directing a retrovirus or lentivirus to infect, or liposome to fuse with a target cell, and many other applications.

### SUMMARY OF THE INVENTION

The present invention provides recombinant viral particles for gene therapy and liposome compositions for drug delivery comprising a receptor-binding sequence of an envelope (env) protein, mutants thereof, nucleic acids encoding same, proteins and compositions comprising same. The invention also provides methods for enhancing delivery of a nucleic acid or compound of interest to a target cell, and methods for targeting same.

In one embodiment, the present invention provides an isolated nucleic acid encoding for a RBM of an MMTV env protein, the isolated nucleic acid having a nucleotide sequence selected from the sequences set forth in SEQ ID No 3-8, 17, 18, and 26.

In another embodiment, the present invention provides an isolated nucleic acid encoding an MMTV env protein, the isolated nucleic acid comprising a mutation in a RBM (RBM) of the env protein, the RBM having a nucleic acid sequence selected from the sequences set forth in SEQ ID No 3-8, 17, 18, and 26.

In another embodiment, the present invention provides a method for delivering a nucleic acid of interest or compound of interest to a target cell, comprising contacting the target cell with a recombinant viral particle or liposome comprising (a) nucleic acid of interest or compound of interest; and (b) a mutated retroviral or lentiviral env protein comprising a heterologous peptide; whereby the heterologous peptide mediates uptake of the recombinant viral particle or liposome via a cellular target molecule, thereby delivering a nucleic acid of interest to a target cell.

In another embodiment, the present invention provides a method for enhancing an ability of a recombinant retroviral or lentiviral particle to infect a target cell, comprising contacting the target cell with an inhibitor of a lysosomal protease, whereby the inhibitor of a vacuolar enzyme prevents or impedes intracellular degradation of the recombinant retroviral or lentiviral particle, thereby enhancing delivery of a recombinant retroviral or lentiviral particle to a target cell.

In another embodiment, the present invention provides an isolated nucleic acid encoding for a heparin-binding motif of an MMTV env protein, the isolated nucleic acid having a nucleotide sequence selected from the sequences set forth in SEQ ID No 27-32, 56-61, and 82.

In another embodiment, the present invention provides recombinant nucleic acid molecule comprising a heterologous nucleotide, die heterologous nucleotide corresponding to an isolated nucleic acid of the present invention.

In another embodiment, the present invention provides an isolated nucleic acid encoding for a RBM of an MoMLV env protein, the isolated nucleic acid having a nucleotide sequence selected from the sequences set forth in SEQ ID No 70-75.

In another embodiment, the present invention provides a recombinant nucleic acid molecule comprising a heterologous nucleotide, the heterologous nucleotide corresponding to an isolated nucleic acid of the present invention.

In another embodiment, the present invention provides an isolated nucleic acid encoding a mutated MoMLV env protein, the isolated nucleic acid comprising a mutation in a RBM of the env protein, the RBM having a nucleic acid sequence selected from the sequences set forth in SEQ ID No 70-75.

In another embodiment, the present invention provides a method for delivering a nucleic acid of interest to a target cell, comprising contacting the target cell with a recombinant viral particle comprising a nucleic acid of interest and the isolated polypeptide of the present invention, whereby the isolated polypeptide mediates uptake of the recombinant viral particle via a cellular molecule, thereby delivering a nucleic acid of interest to a target cell.

In another embodiment, the present invention provides a method for delivering a nucleic acid of interest or compound of interest to a target cell via a clathrin-independent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-dependent endocytosis, and wherein the mutated version of a wild-type env protein comprises an insertion of a heterologous peptide that binds a cellular surface protein that capable of being internalized via a clathrin-independent endocytosis, thereby delivering a nucleic acid of interest or compound of interest to a target cell via a clathrin-independent endocytosis.

In another embodiment, the present invention provides a method for delivering a nucleic acid of interest or compound of interest to a target cell via a clathrin-dependent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-independent endocytosis, and wherein the mutated version of a wild-type env protein comprises an insertion of a heterologous peptide that binds a cellular surface protein that capable of being internalized via a clathrin-dependent endocytosis, thereby delivering a nucleic acid of interest or compound of interest to a target cell via a clathrin-dependent endocytosis.

### BRIEF DESCRIPTION OF THE DRAWINGS

FIG. 1. Diagram of retroviral env proteins. Retroviral env proteins consist of two subunits: the surface protein (SU) and the transmembrane (TM) protein.

FIG. 2. Domains of ecotropic MLV env protein: N, amino-terminus; SP, cleaved signal peptide; VRA, variable region A; VRB, variable region B; PRR, proline rich region; FP, fusion peptide; N—HR, amino-terminal heptad repeat; C—HR, carboxy-terminal heptad repeat; TD, transmembrane anchor domain: R, R-peptide; C, carboxy-terminus; NTD, N-terminal domain; S—S, disulfide bond.

FIG. 3. Insertion of an Sst peptide in the RBM of MoMLV env protein confers upon pseudotyped virus ability to infect cells expressing human somatostatin receptor (SstR). (A) Domain structure of mutant MoMLV env proteins. (B). Infection of SstR-transfected cells by recombinant viral particles containing wild-type MoMLV, MoMLV-Sst-RBM1, MoMLV-Sst-PRR, and MoMLV-Sst-230 env proteins. (C). SstR expression in SstR-transfected cells.

FIG. 4. Infection by MoMLV-Sst-RBM1 recombinant viral particle is mediated by interaction with SstR on target cells. Graph depicts the number of SstR-transfected cells infected by the Sst-RBM1 recombinant viral particle in the presence of soluble recombinant somatostatin (Sst-14), expressed as a percentage of the number of cells infected in the absence of Sst-14.

FIG. 5. Entry into cells through the natural MoMLV receptor is abrogated by the insertion of Sst peptide in the RBM of MoMLV env protein. Efficiency of infection of NIH 3T3 cells by wild-type MoMLV, MoMLV-Sst-RBM1, MoMLV-Sst-PRR, MoMLV-Sst-230, and recombinant viral particle lacking env protein.

FIG. 6. MoMLV-Sst-RBM1 viral particles are unable to infect human neuroblastoma cells due to cathepsin activation. A. Infection by MoMLV-Sst-RBM1 of SstR-transfected cells, SK-N-SH cells, and NB 1643 cells. B. Infection of NIH 3T3 cells by wild-type MoMLV in the presence of an inhibitor of cathepsins B, S, and L, expressed as a percentage of the number of cells infected in the absence of cathepsin inhibitor FIG. 7. Identification of an HBM and an RBM in MMTV SU via sequence and structural alignment with other proteins. (A). Structural alignment of MMTV and F-MLV env protein sequences. The RBM (residues 34-47) is shaded, and the HBM in MMTV and F-MLV env are boxed (residues 122-130 in MMTV) are boxed. β strands, α helices and 3₁₀ helical turns identified in the F-MLV crystal structure are underlined and numbered. Variable regions that change with tropism of the different MLVs (VR regions) are marked by dotted lines. N-linked glycosylation sites are in italics. An arrowhead marks the Arg codon at position 246 used to define the end of the proline-rich region in the MMTV env. (B). Three-dimensional model of MMTV and F-MLV SU proteins, based on secondary structure predicted by sequence of MMTV env. Left panel: Structure of the F-MLV receptor binding domain depicted from the crystal coordinates (Protein Data Base 1AOL), Center panel: Model of the MMTV receptor-binding domain generated using Swiss Model. Right panel: Modified model illustrating potential disulfide bonds between cysteines 62 and 73 and cysteines 133 and 156. The five amino acids comprising the putative RBM of MMTV (residues 40-44) and the amino acid residues identified by mutation analysis to be involved in F-MLV SU/receptor interaction (see FIG. 1) are shown as circled group of space-filled atoms. Long arrows indicate the N-linked glycosylation sites in F-MLV and putative sites in MMTV, which are depicted as space-filled atoms. Dark and light short arrows indicate cysteine residues with potential for stabilizing putative VRA loop and VRC loops, respectively, with disulfide bonds. Abbreviations: N, amino terminus; C, carboxyl terminus; HBM, heparin-binding motif: Structures in the left and center panels were depicted using RasMol 2.7.1.1 (Bernstein, H J et al, Trends Biochem Sci. 25: 453-455, 2000; Sayle, R A et al., Trends Biochem Sci. 20: 374, 1995). Diagram in the right panel was drawn from the RasMol depiction in the center panel. (C). Amino acid sequence comparison of two isolates of wild-type MMTV (RIII and C3H), an MMTV virus adapted to the breast cancer cell line (the RIIIM strain), and two MMTV-like elements (h-MTVs) isolated from primary breast cancer samples. The RBM is boxed, D. Domain structure of the MMTV C3H envelope protein. The signal peptide sequence appears in italics. The receptor binding motif appears in bolded text. The heparin binding motif is highlighted in gray. The proline rich region is underlined with a wavy line. The transmembrane anchor sequence is underlined with a solid line. The sequence encoded by nucleotides of env gene that overlap with the nucleotide coding sequence of sag gene is underlined with a dashed line

FIG. 8. The HBM of MMTV env is not necessary for virus infection. (A). Infection levels of NMuMG cells by recombinant viral particles comprising a wild-type MMTV env (WT), env with a mutated HBM (HBM_{K-A}), or no env protein (pcDNA). The titer for each virus was calculated (LFU/ml) and is presented as the percent wild type infection levels. (B). Expression of WT and HBM_{K-A} env proteins in recombinant viral particles, mock-infected cells (M), or purified virus (V). Supernatants from equal numbers of 293T cells co-transfected with pENV_{C3H} or HBM_{K-A}, pHIT111 and pHIT60 were pelleted by centrifugation through 30% sucrose, then the pellets (supernatant) or the transfected cell extracts (intracellular) were subjected to SDS-PAGE followed by Western blot analysis, using anti-gp52 antisera Arrow shows env (gp52); upper band in the extract lanes is unprocessed env polyprotein. Abbreviations: M, mock-infected; V, purified virus. (C). Relative infectivity of recombinant viral particles comprising a wild-type MMTV env (solid bars) or an env with the heparin-binding motif deleted (open bars), in the presence of heparan sulfate, presented as the percent wild type infection levels without heparan sulfate. Closed bars: wild type pseudovirus; open bars, HBM_{K-A}.

FIG. 9. The RBM of MMTV env is necessary for infectivity. A. Virion protein expression in pseudotyped viruses comprising wild-type MMTV env protein (pENV), or env protein with indicated point mutations. Arrows point to SU (gp52) and TM (gp36). B. Infection efficiency of NMuMG cells by MMTV pseudotyped viruses. Data is presented as the titer (LFU/ml) with the standard deviation for each infection.

FIG. 10. The RBM of MMTV env is necessary for virus binding to mouse cells. (A-B). Histograms showing the binding of wild-type virus (thick line), or Ser₄₀ mutant virus (thin line) to NMuMG cells in the absence (A) and presence (B) of 100 μg/ml heparin sulfate. NMuMG cells were incubated with MMTV pseudotypes, stained with anti-MMTV antisera and FITC-labeled secondary antibodies, and subjected to FACS analysis. Inset shows a Western blot of 10 μl each of concentrated virus preparation or milk-borne MMTV (MMTV) as a positive control. Abbreviations: NV, no virus.

FIG. 11. The RBM of MMTV env is necessary for virus binding to mouse TfR1. The effect of MMTV recombinant viral particles on surface staining of the transferrin receptor was measured using fluorescence antibody. Depicted is mean channel fluorescence value±standard deviation. Abbreviations: 293T, untransfected control; TRH3— virus, transfected 293 cells, no virus: wt, transfected cells, wild-type MMTV_{C3H} virus; Ser40, transfected cells, wild-type MMTV_{C3H} Ser₄₀ virus; wt αMMTV, transfected cells, wild-type MMTV_{C3H} virus that was pre-incubated with anti-MMTV serum; αTfR, transfected cells incubated with anti-TfR monoclonal antibody and wild-type MMTV_{C3H} virus.

FIG. 12. Monoclonal antibodies that block infection recognize the RBM. (A). Efficiency of infection of NMuMG cells by wild-type MMTV_{C3H} virus in the presence of various monoclonal anti-SU antibodies, expressed as a percentage of infection without antibody +/− standard deviation. (B). Recognition of RBM-GST fusion protein by monoclonal antibodies: Lane 1, GST alone; Lane 2, RBM-GST fusion protein; Lane 3, extract from cells transfected with wild-type MMTV env.

FIG. 13. Alignment of the nucleotide sequences encoding nucleotides 8403-8637 and amino acid residues 518-590 of the MMTV C3H env gene and protein (SEQ ID No 43 and 44, respectively; top 2 lines) with the nucleotide sequence encoding amino acid residues 565-632 of the mature Mo-MLV env protein and the nucleotide sequence encoding them in Mo-MLV env gene (SEQ ID No 45 and 46, respectively; bottom two lines). The transmembrane anchor sequence is underlined. The termination or stop codon for the envelope protein is bolded. The naturally occurring Bgl II and Avr II restriction endonuclease recognition sites are indicated in italics and underlining. Note that the Avr II recognition site includes the sequence of the termination or stop codon for the MMTV C3H envelope protein. MMTV C3H full-length sequences (SEQ ID No 2 and 10) are from Genbank Accession number AF228552, and Moloney full-length MLV sequences (SEQ ID No 39 and 40) are from Genbank Accession number MLMCG.

FIG. 14. The R95D mutation increased targeted infection of MoMLV-Sst-RBM1.

FIG. 15. (A) MoMLV-Sst-RBM1 efficiently infects human embryonic kidney 293 cells stably expressing SSTR2a. (B). Stable expression of SSTR2a on HEK 293 cells.

FIG. 16. Flow cytometry of HEK 293 cells stably expressing different SSTR shows that each target receptor type is present on the cell surface in similar numbers.

FIG. 17. MoMLV-Sst-RBM1 can be internalized via somatostatin receptors that are internalized by either clathrin mediated endocytosis or non-clathrin mediated endocytosis.

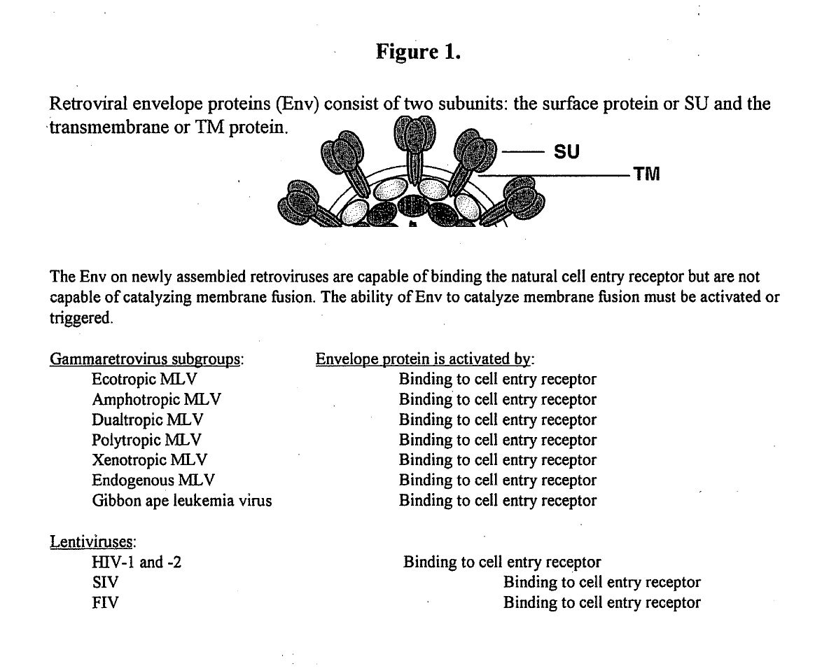

### DETAILED DESCRIPTION OF THE PRESENT INVENTION

The present invention provides recombinant viral particles for gene therapy and liposome compositions for drug delivery comprising a receptor-binding sequence of an envelope (env) protein, mutants thereof, nucleic acids encoding same, proteins and compositions comprising same. The invention also provides methods for enhancing delivery of a nucleic acid or compound of interest to a target cell, and methods for targeting same. In another embodiment, the present invention provides an isolated nucleic acid encoding for a RBM of an MoMLV env protein, the isolated nucleic acid having a nucleotide sequence selected from the sequences set forth in SEQ ID No 70-75.

In one embodiment, the present invention provides an isolated nucleic acid encoding for a RBM of an MMTV env protein, the isolated nucleic acid having a nucleotide sequence selected from the sequences set forth in:

(SEQ ID No 3)

TTTCACGGGTTTAGA.

(SEQ ID NO 4)

GACTTTCACGGGTTTAGAAAC.

(SEQ ID NO 5)

CCTGACTTTCACGGGTTTAGAAACATG.

(SEQ ID NO 6)

TCGCCTGACTTTCACGGGTTTAGAAACATGTCTGGC.

(SEQ ID NO 7)

GGGTCGCCTGACTTTCACGGGTTTAGAAACATGTCTGGC.

(SEQ ID No 8)

GGTGGGTCGCCTGACTTTCACGGGTTTAGAAACATGTCTGGC.

(SEQ ID NO 17)

CAAACCATATATTTGGGTGGGTCGCCTGACTTTCACGGGTTTAGAAACAT
GTC

(SEQ ID NO 18)

GGTGGGTCGCCTGACTTTCACGGGTTTAGAAACATGTCTGGCAATGTACA
TTTTGAGGGGAAGTCTGATACGCTCCCCATTTGCTTTTCCTTCTCCTTTT
CTACCCCCACAGGCTGC

(SEQ ID No 26)

GGTGGGTCGCCTGACTTTCACGGGTTTAGAAACATGTCTGGCAATGTACA
TTTTGAGGGGAAGTCTGATACGCTCCCCATTTGCTTTTCCTTCTCCTTTT
CTACCCCACAGGCTGC

nucleicIn another embodiment, the sequence of the isolated nucleic acid encoding for a RBM is homologous to a nucleotide sequence selected from SEQ ID No 3-8, 17, 18, and 26. In another embodiment, the present invention provides a recombinant nucleic acid molecule comprising a heterologous nucleotide, the heterologous nucleotide corresponding to an isolated nucleic acid encoding for a RBM of the present invention in another embodiment, the present invention provides an isolated polypeptide that functions as an RBM, the isolated polypeptide encoded for by an isolated nucleic acid of the present invention. In another embodiment, the present invention provides an isolated nucleic acid encoding an MMTV env protein, the isolated nucleic acid comprising a mutation in a RBM of the env protein, the RBM having a nucleic acid sequence selected from the sequences set forth in SEQ ID No 3-8, 17, 18, and 26. In another embodiment, the present invention provides a recombinant viral particle, comprising (a) an isolated polypeptide isolated polypeptide of the present invention that functions as an RBM; and (b) a heterologous nucleic acid of interest.

The following is a listing of the sequences in this document and their SEQ ID Nos:

| SEQ ID No: | Type | Name | Description |
|---|---|---|---|
| 1 | NT | Sst | Somatostatin |
| 2 | NT | MMTV env | Entire protein |
| 3-8, | NT | MMTV RBM | Receptor binding motif |
| 17-18, 26 | | | |
| 9 | AA | Sst | Somatostatin |
| 10 | AA | MMTV env | Entire protein |
| 11-16, | AA | MMTV RBM | Receptor binding motif |
| 51-55 | | | |
| 19-25 | AA | Epitope tags | As described below |
| 27-32, | NT | MMTV HBD | Heparin binding domain |
| 56-61, 82 | | | |
| 33-38, | AA | MMTV HBD | Heparin binding domain |
| 62-69 | | | |
| 39 | NT | MoMLV env | Entire protein |
| 40 | AA | MoMLV env | Entire protein |
| 41 | NT | MoMLV-Sst- | Mutated env with Sst |
| | | RBM1 | insertion. |
| 42 | AA | MoMLV-Sst- | Mutated env with Sst |
| | | RBM1 | insertion. |
| 43 | NT | MoMLV tail | Sequence including tail region |
| 44 | AA | MoMLV tail | Sequence including tail region |
| 45 | AA | MMTV tail | Sequence including tail region |
| 46 | NT | MMTV tail | Sequence including tail region |
| 47-48 | NT | Sequencing | As described below |
| | | primers | |
| 49, 70-75 | NT | MoMLV RBM | Receptor binding motif |
| 50, 76-81 | AA | MoMLV RBM | Receptor binding motif |
| 83-86 | NT | Subcloning | As described below |
| | | primers | |
| 87 | AA | Sst-PRR | Mutant MoMLV env protein |
| 88 | AA | MoMLV | Mutant MoMLV env protein |
| | | Sst-230 | |
| 89 | AA | MoMLV Sst-N | Mutant MoMLV env protein |

In one embodiment, the present invention provides an isolated nucleic acid comprising a nucleic acid sequence encoding for an RBM of an MoMLV env protein, corresponding to a nucleotide sequence selected from:

(SEQ ID No 70)

TGTTGCTCAGGGGGCAGCAGCCCAGGCTGTTCCAGAGACTGC.

(SEQ ID No 71)

TGTTCCAGAGACTGC.

(SEQ ID No 72)

TGTATGTTAGCCCACCATGGACCATCTTATTGGGGGCTAGAATATCAATC
CCCTTTTTCTTCTCCCCCGGGGCCCCCTTGTTGCTCAGGGGGCAGCAGCC
CAGGCTGTTCCAGAGACTGCGAAGAACCT.

(SEQ ID No 73)

CCATCTTATTGGGGGCTAGAATATCAATCCCCTTTTTCTTCTCCCCCGGG
GCCCCCTTGTTGCTCAGGGGGCAGCAGCCCAGGCTGTTCCAGAGACTGCG
AAGAACCTTTAACCTCCCTCACCCCTCGGTGC.

(SEQ ID No 74)

CTAGAATATCAATCCCCTTTTTCTTCTCCCCCGGGGCCCCCTTGTTGCTC
AGGGGGCAGCAGCCCAGGCTGTTCCAGAGACTGCGAAGAACCTTTAACCT
CCCTCACCCCTCGGTGC.

(SEQ ID No 75)

TGTATGTTAGCCCACCATGGACCATCTTATTGGGGGCTAGAATATCAATC
CCCTTTTTCTTCTCCCCCGGGGCCCCCTTGTTGCTCAGGGGGCAGCAGCC
CAGGCTGTTCCAGAGACTGCGAAGAACCTTTAACCTCCCTCACCCCTCGG
TGC.

In another embodiment, the sequence of the isolated nucleic acid corresponds to a nucleotide sequence selected from SEQ ID No 70-75. In another embodiment, the sequence of the isolated nucleic acid is homologous to a nucleotide sequence selected from SEQ ID No 70-75.

In another embodiment, the present invention provides a recombinant nucleic acid molecule comprising a heterologous nucleotide, the heterologous nucleotide corresponding to an isolated nucleic acid of the present invention that encodes a MoMLV RBM.

In another embodiment the present invention provides an isolated nucleic acid encoding a mutated MoMLV env protein, the isolated nucleic acid comprising a mutation in a RBM of the env protein, the RBM having a nucleic acid sequence selected from the sequences set forth in SEQ ID No 70-75.

In another embodiment, the present invention provides a method for delivering a nucleic acid of interest to a target cell, comprising contacting the target cell with a recombinant viral particle comprising a nucleic acid of interest and a mutated MoMLV env protein of the present invention, whereby the mutated MoMLV env protein mediates uptake of the recombinant viral particle via a cellular molecule, thereby delivering a nucleic acid of interest to a target cell.

Env proteins mediate binding and/or entry of retroviruses and lentiviruses into cells in the infection process. An RBM is, in one embodiment, a region of an env protein that mediates interaction with a viral receptor. In another embodiment, an RBM mediates entry into a target cell. In another embodiment, an RBM mediates binding to a target cell.

In one embodiment, a viral receptor is a molecule that participates in entry of a viral particle into a target cell. In another embodiment, a viral receptor binds to or interacts with the viral particle, facilitating entry by a different cellular molecule. In another embodiment, a viral receptor binds to or interacts with the viral particle without facilitating entry. In one embodiment, the viral receptor is on the surface of the target cell. In another embodiment, the viral receptor resides in an internal membrane of the target cell.

In one embodiment, a cellular molecule is any molecule inside, on the surface of, or associated with a cell. In another embodiment, a cellular molecule is any molecule produced by a cell. In another embodiment, a cellular molecule is a molecule introduced into a cell from an external source. Each cellular molecule represents a separate embodiment of the present invention.

In one embodiment, the RBD is the domain mediating the receptor binding functions. In another embodiment, the terms “RBD” and “RBM” are used interchangeably. In one embodiment, an RBM of the present invention is constitutive. In another embodiment, an RBM of the present invention is inducible; e.g. by allosteric activation of the env protein. In another embodiment, an RBM of die present invention is any other type of RBM known in the art. Each possibility represents a separate embodiment of the present invention.

In one embodiment, the terms “enter” and “infect” are used interchangeably and refer to entry of a virus or recombinant viral particle into a cell. In another embodiment, the term refers to a process also encompassing one or more events subsequent to entry, such as translocation to the nucleus of the cell, replication of a nucleic acid comprising part of the virus or viral particle, integration of a copy of the virus genome into chromosomes, or expression of the nucleic acid. In another embodiment, the term refers to replication of the virus or recombinant viral particle inside the cell. The term is applicable whether the virus or recombinant viral particle is a wild-type virus or recombinant virus, and applies whether or not the virus is replication competent.

Many techniques are known in the art for measuring in a quantitative or qualitative sense the ability of a virus to infect cells. In one embodiment, infection is measured by assaying the expression of a viral product by one of the techniques for measuring protein expression described herein. In another embodiment, infection is measured by assaying the level of a viral nucleic acid inside the cells or in the supernatant. In another embodiment, infection is measured by assaying the expression of a heterologous marker gene that has been subcloned into the virus (Example 1). Techniques for measuring viral infection are well known in the art, and are described, for example, in Seisenberger G et al, Science. 294: 1929-32 (2001); Loew R et al. Mol Ther. 9: 738-46 (2004); Bower J F et al, J Virol 78: 4710-9 (2004); Barin F et al, J. Infect. Dis. 189: 322-7 (2004); Yang, X et al, J Virol 75: 1165-71 (2001); DiStefano D J et al, J Virol Methods 55: 199-208 (1995); Usuba O et al, Viral Immunol 3: 237-41 (1990); Robb H A, Virology 41: 761-2 (1970); and Scotti P D, J Gen Virol 35: 393-6 (1977). Each such technique represents a separate embodiment of the present invention.

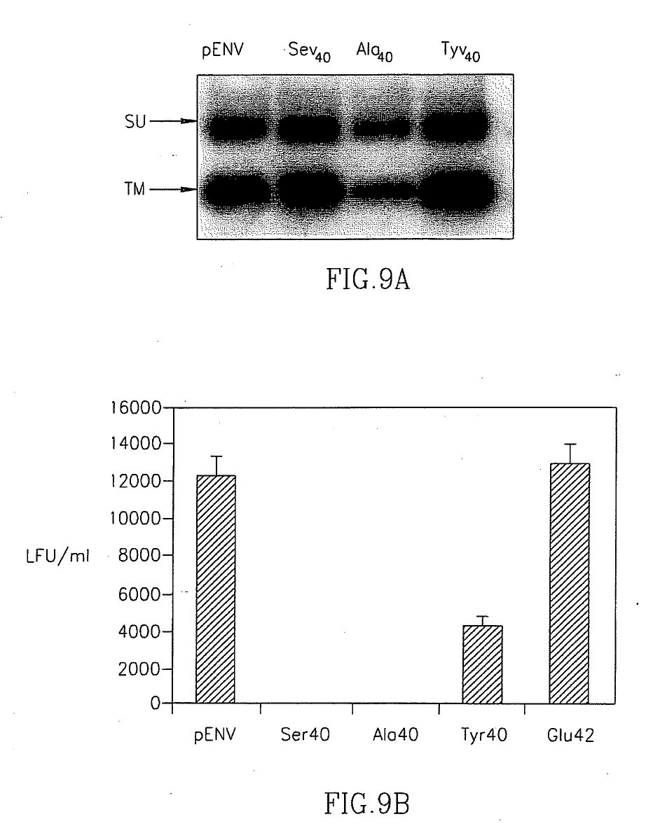

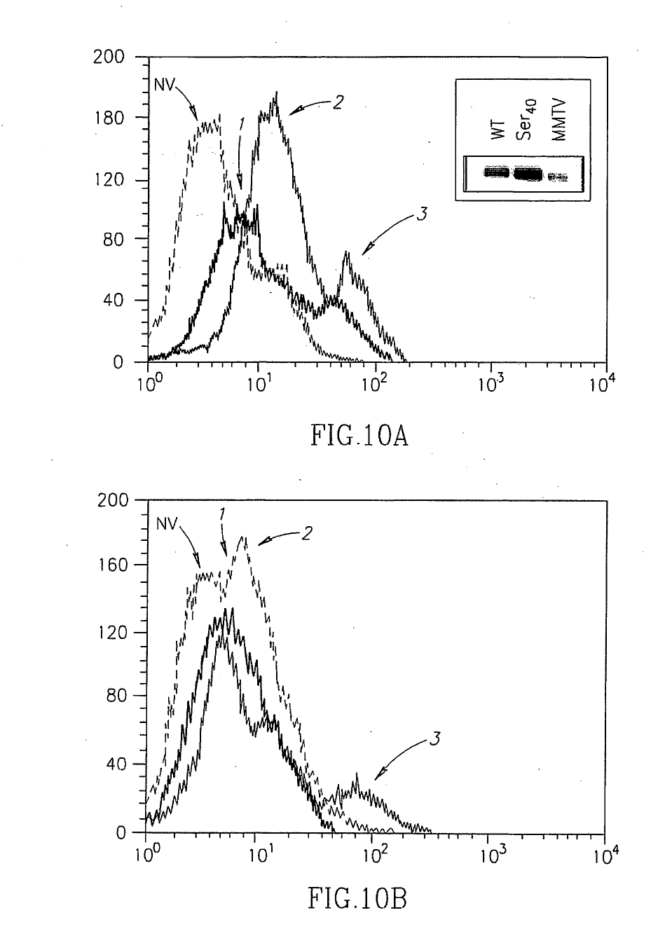

MMTV enters cells via an interaction between MMTV en, protein and the viral receptor TfR1. MMTV env, is produced as a polyprotein precursor, and is cleaved after synthesis into surface protein (SU) and transmembrane protein (TM). The present invention has delineated the RBM of MMTV env protein (FIG. 7), and has shown that the RBM is necessary for binding to mouse cells (FIG. 10) and for infection (FIG. 9).

In one embodiment, the sequence encoding for an RBM is part of a gene encoding an MoMLV env protein or a homologue thereof. The MoMLV env gene has, in one embodiment, the following sequence:

ATGGCGCGTTCAACGCTCTCAAAACCCCTTAAAAATAAGGTTAACCCGCG
AGGCCCCCTAATCCCCTTAATTCTTCTGATGCTCAGAGGGGTCAGTACTG
CTTCGCCCGGCTCCAGTCCTCATCAAGTCTATAATATCACCTGGGAGGTA
ACCAATGGAGATCGGGAGACGGTATGGGCAACTTCTGGCAACCACCCTCT
GTGGACCTGGTGGCCTGACCTTACCCCAGATTTATGTATGTTAGCCCACC
ATGGACCATCTTATTGGGGGCTAGAATATCAATCCCCTTTTTCTTCTCCC
CCGGGGCCCCCTTGTTGCTCAGGGGGCAGCAGCCCAGGCTGTTCCAGAGA
CTGCGAAGAACCTTTAACCTCCCTCACCCCTCGGTGCAACACTGCCTGGA
ACAGACTCAAGCTAGACCAGACAACTCATAAATCAAATGAGGGATTTTAT
GTTTGCCCCGGGCCCCACCGCCCCCGAGAATCCAAGTCATGTGGGGGTCC
AGACTCCTTCTACTGTGCCTATTGGGGCTGTGAGACAACCGGTAGAGCTT
ACTGGAAGCCCTCCTCATCATGGGATTTCATCACAGTAAACAACAATCTC
ACCTCTGACCAGGCTGTCCAGGTATGCAAAGATAATAAGTGGTGCAACCC
CTTAGTTATTCGGTTTACAGACGCCGGGAGACGGGTTACTTCCTGGACCA
CAGGACATTACTGGGGCTTACGTTTGTATGTCTCCGGACAAGATCCAGGG
CTTACATTTGGGATCCGACTCAGATACCAAAATCTAGGACCCCGCGTCCC
AATAGGGCCAAACCCCGTTCTGGCAGACCAACAGCCACTCTCCAACGCCC
AAACCTGTTAAGTCGCCTTCAGTCACCAAACCACCCAGTGGGACTCCTCT
CTCCCCTACCCAACTTCCACCGGCGGGAACGGAAAATAGGCTGCTAAACT
TAGTAGACGGAGCCTACCAAGCCCTCAACCTCACCAGTCCTGACAAAACC
CAAGAGTGCTGGTTGTGTCTAGTAGCGGGACCCCCCTACTACGAAGGGGT
TGCCGTCCTGGGTACCTACTCCAACCATACCTCTGCTCCAGCCAACTGCT
CCGTGGCCTCCCAACACAAGTTGACCCTGTCCGAAGTGACCGGACAGGGA
CTCTGCATAGGAGCAGTTCCCAAAACACATCAGGCCCTATGTAATACCAC
CCAGACAAGCAGTCGAGGGTCCTATTATCTAGTTGCCCCTACAGGTACCA
TGTGGGCTTGTAGTACCGGGCTTACTCCATGCATCTCCACCACCATACTG
AACCTTACCACTGATTATTGTGTTCTTGTCGAACTCTGGCCAAGAGTCAC
CTATCATTCCCCCAGCTATGTTTACGGCCTGTTTGAGAGATCCAACCGAC
ACAAAAGAGAACCGGTGTCGTTAACCCTGGCCCTATTATTGGGTGGACTA
ACCATGGGGGGAATTGCCGCTGGAATAGGAACAGGGACTACTGGTCTAAT
GGCCACTCAGCAATTCCAGCAGCTCCAAGCCGCAGTACAGGATGATCTCA
GGGAGGTTGAAAAATCAATCTCTAACCTAGAAAAGTCTCTCACTTCCCTG
TCTGAAGTTGTCCTACAGAATCGAAGGGGCCTAGACTTGTTATTTCTAAA
AGAAGGAGGGCTGTGTGCTGCTCTAAAAGAAGAATGTTGCTTCTATGCGG
ACCACACAGGACTAGTGAGAGACAGCATGGCCAAATTGAGAGAGAGGCTT
AATCAGAGACAGAAACTGTTTGAGTCAACTCAAGGATGGTTTGAGGGACT
GTTTAACAGATCCCCTTGGTTTACCACCTTGATATCTACCATTATGGGAC
CCCTCATTGTACTCCTAATGATTTTGCTCTTCGGACCCTGCATTCTTAAT
CGATTAGTCCAATTTGTTAAAGACAGGATATCAGTGGTCCAGGCTCTAGT
TTTGACTCAACAATATCACCAGCTGAAGCCTATAGAGTACGAGCCA
(SEQ ID No:39, GenBank accession # MLMCG) (Example
1), or, in other embodiments, to one of the se-
quences set forth in GenBank accession # J02256,
J02257, or M76668.

In another embodiment, the MoMLV env gene has a sequence homologous to one of the above sequences.

In another embodiment, the nucleic acid encoding for an RBM corresponds to nucleotides 313-354 of SEQ ID No 39 (TGTTGCTCAGGGGGCAGCAGCCCAGGCTGTTCCAGAGACTGC, SEQ ID No 49), or a fragment thereof.

In one embodiment, the sequence encoding for an RBM comprises part of a gene encoding an MMTV env protein or a homologue thereof. The MMTV env gene has, in one embodiment, the following sequence:

ATGCCGAAACACCAATCTGGGTCCCCGATCGGTTCATCCGACCTTTTACT
GAGCGGAAAGAAGCAACGCCCACACCTGGCACTGCGGAGAAAACGCCGCC
GCGAGATGAGAAAGATCAACAGAAAAGTCCGGAGGATGAATCTAGCCCCC
ATCAAAGAGAAGACGGCTTGGCAACATCTGCAGGCGTTAATCTTCGAAGC
GGAGGAGGTTCTTAAAACCTCACAAACTCCCCAAACCTCTTTGACTTTAT
TTCTTGCTTTGTTGTCTGTCCTCGGCCCCCCGCCTGTGACCGGGGAAAGT
TATTGGGCTTACCTACCTAAACCACCTATTCTCCATCCCGTGGGATGGGG
AAATACAGACCCCATTAGAGTTCTGACCAATCAAACCATATATTTGGGTG
GGTCGCCTGACTTTCACGGGTTTAGAAACATGTCTGGCAATGTACATTTT
GAGGGGAAGTCTGATACGCTCCCCATTTGCTTTTCCTTCTCCTTTTCTAC
CCCCACAGGCTGCTTTCAAGTAGATAAGCAAGTATTTCTTTCTGATACAC
CCACGGTTGATAATAATAAACCTGGGGGAAAGGGTGATAAAAGGCGTATG
TGGGAACTCTGGTTGACTACTTTGGGGAACTCAGGGGCCAATACAAAACT
GGTCCCTATAAAGAAGAAGTTGCCCCCCAAATATCCTCACTGCCAGATCG
CCTTTAAGAAGGACGCCTTCTGGGAGGGAGACGAGTCTGCTCCTCCACGG
TGGTTGCCTTGCGCCTTCCCTGACCAGGGGGTGAGTTTTTCTCCAAAAGG
GGCCCTTGGGTTACTTTGGGATTTCTCCCTTCCCTCGCCTAGTGTAGATC
AGTCAGATCAGATTAAAAGCAAAAAGGATCTATTTGGAAATTATACTCCC
CCTGTCAATAAAGAGGTTCATCGATGGTATGAAGCAGGATGGGTAGAACC
TACATGGTTCTGGGAAAATTCTCCTAAGGATCCCAATGATAGAGATTTTA
CTGCTCTAGTTCCCCATACAGAATTGTTTCGCTTAGTTGCAGCCTCAAGA
TATCTTATTCTCAAAAGGCCAGGATTTCAAGAACATGACATGATTCCTAC
ATCTGCCTGTGTTACTTACCCTCATGCCATATTATTAGGATTACCTCAGC
TAATAGATATAGAGAAAAGAGGATCTACTTTTCATATTTCCTGTTCTTCT
TGTAGATTGACTAATTGTTTAGATTCTTCTGCCTACGACTATGCAGCGAT
CATAGTCAAGAGGCCGCCATACGTGCTGCTACCTGTAGATATTGGTGATG
AACCATGGTTTGATGATTCTGCCATTCAAACCTTTAGGTATGCCACAGAT
TTAATTCGAGCCAAGCGATTCGTCGCTGCCATTATTCTGGGCATATCTGC
TTTAATTGCTATTATCACTTCCTTTGCTGTAGCTACTACTGCTTTAGTTA
AGGAGATGCAAACTGCTACGTTTGTTAATAATCTTCATAGGAATGTTACA
TTAGCTTTATCTGAACAAAGAATAATAGATTTAAAATTAGAAGCTAGACT
TAATGCTTTAGAAGAAGTAGTTTTAGAGTTGGGACAAGATGTGGCAAACT
TAAAGACCAGAATGTCCACCAGGTGTCATGCAAATTATGATTTTATCTGC
GTTACACCTTTACCATATAATGCTTCTGAGAGCTGGGAAAGAACCAAAGC
TCATTTATTGGGCATTTGGAATGACAATGAGATTTCATATAACATACAAG
AATTAACCAACCTGATTAGTGATATGAGCAAACAACATATTGACACAGTG
GACCTCAGTGGCTTGGCTCAGTCCTTTGCCAATGGAGTAAAGGCTTTAAA
TCCATTAGATTGGACACAATATTTCATTTTTATAGGTGTTGGAGCCCTGC
TTTTAGTCATAGTGCTTATGATTTTCCCCATTGTTTTCCAGTGCCTTGCG
AAGAGCCTTGACCAAGTGCAGTCAGATCTTAACGTGCTTCTTTTAAAAAA
GAAAAAAGGGGGAAATGCCGCGCCTGCAGCAGAAATGGTTGAACTCCCGA
GAGTGTCCTACACC (SEQ ID No 2, GenBank accession #
AF228552).

In another embodiment, the nucleic acid encoding for an RBM corresponds to nucleotides 400-438 of SEQ ID No 2 (set forth above in SEQ ID NO 7). In another embodiment, the nucleotide sequence of the RBM corresponds to SEQ ID No 8.

In another embodiment, the nucleic acid encoding for an RBM corresponds to an RBM of an env gene homologous to SEQ ID No 2 or SEQ ID No 39.

In another embodiment, the MMTV env gene corresponds to or is homologous to an MMTV env nucleotide sequence such as that disclosed in NCBI's Entrez nucleotide database, having the Accession Number: X01811, AF346816, U41642, AF071010, M11024, K00556, BC018102, AF033807, AF263910, AF228551, AF228550. M22028 or M15122.

In one embodiment of the present invention, “nucleic acid” refers to a string of at least two base-sugar-phosphate combinations. The term includes, in one embodiment, deoxyribonucleic acid (DNA) and ribonucleic acid (RNA). “Nucleotide” refers, in one embodiment, to a monomeric unit of a nucleic acid polymer RNA is in the form of a tRNA (transfer RNA), snRNA (small nuclear RNA), rRNA (ribosomal RNA), mRNA (messenger RNA), anti-sense RNA, small inhibitory RNA (siRNA), micro RNA (mRNA) or ribozymes. The use of siRNA and mRNA has been described (Caudy A A et al. Genes & Devel 16:2491-96 (2002), Paddison P J et al., Methods Mol Biol. 265:85-100 (2004), Paddison P J et al., Proc Natl Acad Sci USA. 99:1443-8 (2002) and references cited therein). DNA is in the form of plasmid DNA, viral DNA, linear DNA, or chromosomal DNA or derivatives of these groups. In addition, these forms of DNA and RNA is single, double, triple, or quadruple stranded. The term also includes, in one embodiment, artificial nucleic acids that may contain other types of backbones but the same bases. Examples of artificial nucleic acids are PNAs (peptide nucleic acids), phosphorothioates, and other variants of the phosphate backbone of native nucleic acids. PNA may contain peptide backbones and nucleotide bases, and is able to bind both DNA and RNA molecules. The use of phosphothiorate nucleic acids and PNA are known to those skilled in the art, and are described in, for example, Nielsen P E, Curr Opin Struct Biol 9:353-57 (1999), Nielsen P E., Mol Biotechnol. 26:233-48 (2004), Rebuffat A G et al., FASEB J. 16:1426-8 (2002), Inui T et al., J. Biol. Chem. 272:8109-12 (1997), Chasty R et al., Leuk Res. 20:391-5 (1996) and references cited therein; and Raz N K et al Biocliem Biophys Res Commun. 297:1075-84. In another embodiment, the term includes any derivative of any type of RNA or DNA known in the art. The production and use of nucleic acids is known to those skilled in art and is described, for example, in Molecular Cloning, Sambrook and Russell, eds. (2001), and Methods in Enzymology: Guide to Molecular Cloning Techniques (2001) Berger and Kimmel, eds. Each nucleic acid derivative represents a separate embodiment of die present invention.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences may, in one embodiment, have variations as compared to wild type sequences, nevertheless encoding the protein or peptide of interest, or fragments thereof, retaining wild type function exhibiting the same biological effect, despite these variations. Each fragment, derivative, or variation represents a separate embodiment of this present invention.

The nucleic acids can be produced by any synthetic or recombinant process that is known in the art. Nucleic acids can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the invention can also be chemically synthesized by any method known in the art. For example, the DNA can be synthesized chemically from the four nucleotides in whole or, in part by methods known in the art. Such methods include those described in Caruthers M H. Science 230:281-5 (1985). DNA can also be synthesized by preparing overlapping double-stranded oligonucleotides, filling in the gaps, and ligating the ends together (see, generally, Molecular Cloning (ibid) and Glover R P et al., Rapid Commun Mass Spectrom 9:897-901, 1995). DNA expressing functional homologues of the protein can be prepared from wild-type DNA by site-directed mutagenesis (see, for example, Molecular Biology: Current Innovations and Future Trends. A. M. Griffin and H. G. Griffin, Eds. (1995); and Kim D F et al, Cold Spring Harb Symp Quant Biol. 66:1119-26 (2001). The DNA obtained can be amplified by methods known in the art. One suitable method is the polymerase chain reaction (PCR) method described in Molecular Cloning (ibid). Each of these methods represents a separate embodiment of the present invention.

Methods for modifying nucleic acids to achieve specific purposes are disclosed in the art, for example, in Molecular Cloning (ibid). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention. Each of these methods and variations represents a separate embodiment of the present invention.

In one embodiment, the terms “homology”, “homologue” or “homologous” indicate a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. In another embodiment, the terms indicate that the sequence referred to, whether an amino acid sequence, or a nucleic acid sequence, exhibits at least 70% correspondence with a sequence of the present invention. In another embodiment, the correspondence is at least 72%. In another embodiment, the correspondence is at least 75%. In another embodiment, the correspondence is at least 77%. In another embodiment, the correspondence is at least 80%. In another embodiment, the correspondence is at least 82%. In another embodiment, the correspondence is at least 85%. In another embodiment, the correspondence is at least 87%. In another embodiment, the correspondence is at least 90%. In another embodiment, the correspondence is at least 92%. In another embodiment, the correspondence is at least 95%. In another embodiment, the sequence exhibits 95%-100% correspondence to the indicated sequence. In another embodiment, the reference to a correspondence to a particular sequence includes both direct correspondence, as well as homology to that sequence as herein defined.

Homology is determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of candidate sequence hybridization, methods of which are well described in the art (See, for example, Nucleic Acid Hybridization. Hames and Higgins, Eds. (1985); Molecular Cloning. Sambrook and Russell, eds. (2001), and Current Protocols in Molecular Biology, Ausubel et al. eds, 1989). For example, methods of hybridization is, in one embodiment, carried out under moderate to stringent conditions, to the complement of a DNA encoding a native peptide or protein of interest. Hybridization conditions is, for example, overnight incubation at 42° C. in a solution comprising: 10-20% formamide, 5×SSC (150 millimolar (mM) NaCl, 15 mM trisodium citrate), 50 mM sodium phosphate (pH 7.6), 5× Denhardt's solution, 10% dextran sulfate, and 20 micrograms (μg)/milliliter (ml) denatured, sheared salmon sperm DNA. Each method represents a separate embodiment of the present invention.

In another embodiment, the present invention provides a vector, cell, or packaging cell line comprising any isolated nucleic acid of the present invention. In one embodiment, “vector” refers to a vehicle that facilitates expression of a nucleic acid molecule inserted therein in a cell. In another embodiments a vector facilitates expression in an expression system such as a reticulocyte extract. A vector comprises, in one embodiment, a nucleic acid comprising non-coding nucleic acid sequences or coding sequences other than the inserted nucleic acid.

A large number of vectors known in the art may be used in this embodiment. A vector includes, in some embodiments, an appropriate selectable marker. In other embodiments, the vector further includes an origin of replication, or is a shuttle vector, which can propagate both in bacteria, such as, for example, E. coli (wherein the vector comprises an appropriate selectable marker and origin of replication) or be compatible for propagation in vertebrate cells, or integration in the genome of an organism of choice. The vector according to this aspect of the present invention is, for example, a plasmid, a bacmid, a phagemid, a cosmid, a phage, a modified or unmodified virus, an artificial chromosome, or any other vector known in the art. Many such vectors are commercially available, and their use is well known to those skilled in the art (see, for example, Molecular Cloning, (2001), Sambrook and Russell, eds.). Each vector represents a separate embodiment of the present invention.

In another embodiment, the nucleotide molecule present in the vector is a plasmid, cosmid, or the like, or a vector or strand of nucleic acid. In another embodiment, the nucleotide molecule is genetic material of a living organism, virus, phage, or material derived from a living organism, virus, or phage. The nucleotide molecule is, in one embodiment, linear, circular, or concatemerized, and is of any length. Each tripe of nucleotide molecule represents a separate embodiment of the present invention.

According to another embodiment, nucleic acid vectors comprising the isolated nucleic acid sequence include a promoter for regulating expression of the isolated nucleic acid. Such promoters are known to be cis-acting sequence elements required for transcription, as they serve to bind DNA-dependent RNA polymerase, which transcribes sequences present downstream thereof. Each vector disclosed herein represents a separate embodiment of the present invention.

In one embodiment, the isolated nucleic acid is subcloned into the vector. “Subcloning”, in all the applications disclosed herein, refers, in one embodiment, to inserting an oligonucleotide into a nucleotide molecule. For example, in one embodiment isolated DNA encoding an RNA transcript can be inserted into an appropriate expression vector that is suitable for the host cell such that the DNA is transcribed to produce the RNA.

The insertion into a vector can, in one embodiment, be accomplished by ligating the DNA fragment into a vector that has complementary cohesive termini. However, if the complementary restriction sites used to fragment the DNA are not present in the cloning vector, the ends of the DNA molecules may, in another embodiment, be enzymatically modified. Alternatively, any site desired is produced by ligating nucleotide sequences (linkers) onto the DNA termini: these ligated linkers may comprise specific chemically synthesized oligonucleotides encoding restriction endonuclease recognition sequences Methods for subcloning are known to those skilled in the art, and are described, for example in Molecular Cloning, (2001), Sambrook and Russell, eds. Each of these methods represents a separate embodiment of the present invention.

“Packaging cell line” refers, in one embodiment, to a cell comprising all or a portion of a viral genome and capable of producing viral particles. In one embodiment, the packaging cell line requires that additional viral sequences be supplied exogenously (for example, in a vector, plasmid, or the like) in order to produce viral particles. In another embodiment, the packaging cell line does not require additional viral sequences to produce viral particles. The construction and use of packaging cell lines is well known in the art, and is described, for example, in U.S. Pat. No. 6,589,763 and Kalpana G V et al, Semin Liver Disease 19:27-37 (1999). Each packaging cell line known in the art represents a separate embodiment of the present invention.

In another embodiment, the present invention provides an isolated polypeptide, comprising an RBM of an MMTV env protein. In one embodiment, the RBM has the sequence: FHGFR (SEQ ID No 11). In another embodiment, the RBM has the sequence: DFHGFRN, (SEQ ID No 12); or, in another embodiment, the sequence: PDFHGFRNM, (SEQ ID No 13); or, in another embodiment, the sequence: SPDFHGFRNMS, (SEQ ID No 14); or, in another embodiment, the sequence: SPDFHGFRNMSG, (SEQ ID No 15); or, in another embodiment, the sequence: GGSPDFHGFRNMSG, (SEQ ID No 16); or, in another embodiment, the sequence: LGGSPDFHGFRNMS, (SEQ ID No 51); or, in another embodiment, the sequence: YLGGSPDFH, (SEQ ID No 52); or, in another embodiment, the sequence: QTIYLGGSPDFHGFRNMSG, (SEQ ID No 53); or, in another embodiment, the sequence: GGSPDFHGFRNMSGNVHFEGKSDTLPICFSFSFSTPTGC (SEQ ID No 54); or, in another embodiment, the sequence: QTIYLGGSPDFHGFRNMSGNVHFEGKSDTLPICFSFSFSTPTGC (SEQ ID No 55). In another embodiment, the RBM of the protein is homologous to one of the above sequences.

In another embodiment, the present invention provides an isolated polypeptide, comprising an RBM of an MoMLV env protein. In one embodiment, the RBM corresponds to the following residues of the MoMLV env protein sequence (CCSGGSSPGCSRDC, SEQ ID No 76); or, in another embodiment, CSRDC (SEQ ID No 77); or, in another embodiment, CMLAHHGPSYWGLEYQSPFSSPPGPPCCSGGSSPGCSRDCEEP (SEQ ID No 78); or, in another embodiment, PSYWGLEYQSPFSSPPGPPCCSGGSSPGCSRDCEEPLTSLTPRC (SEQ ID No 79); or, in another embodiment, LEYQSPFSSPPGPPCCSGGSSPGCSRDCEEPLTSLTPRC (SEQ ID No 80); or, in another embodiment, CMLAHHGPSYWGLEYQSPFSSPPGPPCCSGGSSPGCSRDCEEPLTSLTPRC (SEQ ID No 81). In another embodiment, the RBM is homologous to one of the above sequences. In another embodiment, the RBM corresponds to a homologous to identical stretch of an env protein homologous to MoMLV env protein, even if the residue numbers in the homologous env protein are not the same as those in MoMLV env.

In another embodiment, the present invention provides an isolated polypeptide, comprising an RBM of an MoMLV env protein. In another embodiment, the RBM of the protein corresponds to residues 72-85 of SEQ ID No 40 (CCSGGSSPGCSRDC, SEQ ID No 50). In another embodiment, the RBM of the protein is homologous to an RBM sequence of the present invention.

In one embodiment, polypeptides of the present invention include, but are not limited to, fragments of native polypeptides from any animal species, including degradation products, synthetically synthesized peptides or recombinant peptides, variants and derivatives of native polypeptides and their fragments, provided that they have a biological activity in common with a respective native polypeptide. “Fragments” comprise regions within the sequence of a mature native polypeptide. The term “derivative” is meant to include amino acid sequence and glycosylation variants, and covalent modifications of a native polypeptide, whereas the term “variant” refers to amino acid sequence and glycosylation variants within this definition. In another embodiment, the agent of the invention comprises a peptidomimetic (tropically, synthetically synthesized peptides), such as peptoids and semipeptoids which are peptide analogs, which may have, for example, modifications rendering the peptides more stable while in a body or more capable of penetrating into cells. In one embodiment, such modifications include, but are not limited to N-terminal, C-terminal or peptide bond modification, including, but not limited to, backbone modifications, and residue modification, each of which represents an additional embodiment of the invention. Methods for preparing peptidomimetic compounds are well known in the art and are specified, for example, in Quantitative Drug Design, Hansch, Sammes, and Taylor, eds. (1990).

It is to be understood that any peptide of the present invention may, in one embodiment, be isolated, generated synthetically, obtained via translation of sequences subjected to any mutagenesis technique, as well as obtained via any protein evolution techniques, known to those skilled in the art.

Recombinant protein production is one means whereby peptides of the invention are produced. The recombinant proteins are then, in one embodiment, introduced into an organism. Any method of generating proteins or peptides known in the art represents a separate embodiment of the present invention.

Protein expression can be verified, in one embodiment, by methods including, but not limited to, HPLC, mass spectroscopy, GLC, immunohistochemistry, ELISA, RIA, or western blot analysis. When using a method that relies on the immunological properties of the protein in question, antibodies against the entire protein or a peptide derived from the protein can be raised and used. Alternatively, and according to another embodiment of the present invention, an expressed sequence tag (EST) encoding a known tag peptide sequence (for example HIS tag) can be inserted into the recombinant protein either on the 5′ or the 3′ end thus the HIS-tag proteins can be isolated using His-Tag Ni-column chromatography. Similarly, in still another preferred embodiment of the present invention, a polycistronic recombinant nucleic acid including an Internal Ribosome Entry Site (IRES) sequence residing between the sequence encoding the protein of interest and a sequence encoding a reporter protein is generated, so as to enable detection of a known marker protein. Additional marker proteins can be incorporated, or comprise the recombinant proteins, and as such encompass still further preferred embodiments of the present invention. Each method represents a separate embodiment of the present invention.

In another embodiment, the present invention provides a recombinant protein comprising an isolated polypeptide of the present invention. In one embodiment, the recombinant protein comprises sequence from an env protein of a lentivirus. In another embodiment, the recombinant protein comprises sequence from a retrovirus other than MMTV. In another embodiment, the recombinant protein comprises sequence from an env protein of a virus other than a retrovirus or lentivirus. Each protein represents a separate embodiment of the present invention.

In another embodiment, the present invention provides a recombinant viral particle, cell or packaging cell line comprising an isolated polypeptide of the present invention. In another embodiment, the present invention provides a nucleic acid encoding any protein or polypeptide of the present invention, each of which is considered a separate embodiment of the present invention.

In another embodiment, the present invention provides an isolated nucleic acid, the isolated nucleic acid encoding for a mutated RBM of an MoMLV env protein. In another embodiment, the present invention provides an isolated nucleic acid, the isolated nucleic acid encoding for a mutated RBM of an MMTV env protein. In another embodiment, the isolated nucleic acid has the sequence of SEQ ID No 41 (Example 1).

The present invention demonstrated that a mutation in an RBM of an env protein can expand or alter the tropism of a viral particle. A somatostatin (Sst) peptide (YASAGCKNFFWIKTFTSCYTAS, (SEQ ID No 9) was put in place of the RBM of Moloney MLV (MoMLV) env protein, creating the gene MoMLV-Sst-RBM1 (SEQ ID No 41; Example 1). Virus particles comprising MoMLV-Sst-RBM1 were able to infect cells expressing the somatostatin receptor type 2a (SstR2a), which is not usually a receptor for MoMLV. In addition, the ability of MoMLV to enter cells via its usual receptor was abrogated (Example 3). These findings demonstrated that replacement of the RBM of a viral env protein by a heterologous sequence is an effective strategy for changing the tropism of a viral particle. In addition, ability of MoMLV to enter cells via its usual receptor was abrogated (Example 3).

“Tropism,” in one embodiment, refers to the range of cells that a viral particle is capable of infecting. In another embodiment, “tropism” refers to the range of cells that a viral particle is capable of efficiently infecting. In another embodiment, “tropism” refers to the range of cells that a viral particle is capable of entering. In another embodiment, tropism refers to the range of cells that a viral particle is capable of efficiently entering.

In another embodiment, the isolated nucleic acid comprises a deletion of sequence encoding the RBM. In another embodiment, the isolated nucleic acid comprises a substitution (e.g. replacement) of heterologous sequence for all or part of the sequence encoding the RBM. In another embodiment, the isolated nucleic acid comprises an insertion of heterologous sequence into the sequence encoding the RBM. The heterologous sequence need not, in one embodiment, be the same length as the deleted sequence. In another embodiment, the isolated nucleic acid comprises any combination of insertion, deletion, and substitution mutations.

In one embodiment, an insertion, deletion, or substitution of the present invention is in nucleotides 400-438 of a nucleic acid sequence as set forth in SEQ ID No 2. An insertion, deletion, or substitution of the invention may, in one embodiment, encompass either all or part of an RBM. In another embodiment, the insertion, deletion, or substitution may encompass sequence both within and outside the RBM. In another embodiment, the insertion, deletion, or substitution encompasses sequence entirely outside the RBM. In another embodiment, a mutation outside the RBM is combined with a mutation inside the RBM.

In one embodiment, an insertion, deletion, or substitution of the present invention may encompass any number of nucleotides. Each size of insertion, deletion, or substitution represents a separate embodiment of the present invention. Any combination of any of the mutations described herein is included in the present invention.

Another embodiment of the present invention comprises a mutation entirely outside the RBM that indirectly affects the RBM by affecting overall conformation of the protein in such the conformational of the RBM is altered. In another embodiment, the present invention may comprise a point mutation, frameshift mutation, a mutation that introduces a stop codon, or any other type of mutation known in the art. In another embodiment, the present invention may comprise any combination of a mutation of the present invention. Techniques for the introduction of a mutation are well known in the art, for example in Molecular Cloning, (2001), Sambrook and Russell, eds, and in Molecular Biology: Current Innovations and Future Trends, (1995), A M Griffin and H G Griffin, eds. Each mutation, and each technique for introducing a mutation, represents a separate embodiment of the present invention.

The introduction of a mutation is verified, in one embodiment, by a method such as DNA sequencing. In another embodiment, introduction of the mutation is verified by methods including restriction enzyme analysis, electrophoretic mobility assay, tryptic peptide digest of the protein encoded for, altered enzyme activity in a cell-based or a cell-free assay, alteration in substrate or antibody-binding pattern, altered isoelectric point, direct amino acid sequencing, and any other of the known assay techniques useful for detecting mutations in a polynucleotide or protein. Such an assay can be provided in a single detection format or a multi-detection format such as an antibody chip array. Methods for verifying introduction of a mutation are well known in the art, and are described, for example, in Molecular Cloning (2001), Sambrook and Russell, eds, and in Molecular Biology: Current Innovations and Future Trends, (1995), A M Griffin and H G Griffin, eds. Each such method represents a separate embodiment of the present invention.

In another embodiment, the present invention provides an isolated polypeptide, comprising a mutated RBM of an MMTV env protein. In another embodiment, the mutated RBM is produced using a nucleic acid encoding for such. Each nucleic acid of the present invention encoding for a mutated RBM is used, and represents a separate embodiment of the invention.

In another embodiment, the RBM that is mutated corresponds to (SEQ ID No 11); or in another embodiment, (SEQ ID No 12); or in another embodiment, (SEQ ID No 13); or in another embodiment, (SEQ ID No 14); or in another embodiment, (SEQ ID No 15); or in another embodiment, (SEQ ID No 16); or in another embodiment, (SEQ ID No 51); or in another embodiment, (SEQ ID No 52); or in another embodiment, (SEQ ID No 53); or in another embodiment, (SEQ ID No 54); or in another embodiment, (SEQ ID No 55). Each possibility represents a separate embodiment of the present invention.

In another embodiment, the mutated RBM of the present invention comprises an insertion of all or part of a heterologous peptide. In one embodiment, the heterologous peptide interacts with a cellular molecule. In another embodiment, the heterologous peptide does not interact with a cellular molecule.

In one embodiment, the heterologous peptide is a somatostatin peptide (Example 1). In another embodiment, the heterologous sequence has a sequence corresponding to or homologous to the sequence YASAGCKNFFWKTFTSCYTAS (SEQ ID No 9). In another embodiment, the heterologous peptide corresponds to or is homologous to an Sst amino acid sequence or a homologue thereof, e.g. those disclosed in NCBI's Entrez protein database, having the Accession Number: NP_{—}001039. AAH32625, NP_{—}075945, NP_{—}035832, NP_{—}031771, AAH10770, NP_{—}001293, P01166, RIHUS1, P56469, or AAA60566.

Sst is a 14 amino acid peptide hormone that elicits a variety of effects on different cell types expressing a somatostatin receptor (SstR). A number of subtypes and isoforms of SstR, namely sst1, sst2A, sst2B, sst3, sst4, sst5, have been identified (Moller et al, Biochim Biophys Acta 1616:1-84 (2003).

In another embodiment, the heterologous peptide inserted into the RBM is a homologue of analogue of somatostatin-14, such as somatostatin-28, or one of the somatostatin (Sst) analogues cortistatin-14, cortistatin-17, a [Pro2, Met13] sst-28, prosomatostatin, octreotide, lanreotide, vapreotide, MK-678, RC160, SOM230, L-362, 855, BIM 23268, BIM 23052, CH-275, SDZ 222-100, KE 106, PTR 3173, sst 3-ODN-8, CYN-154806, BIM 23056, BIM 23627, SB-710411, PRL-2970, TT-232, Lan-7, KE 108, NVP-SRA880, VIP, 5-HT, or a homologue or variant thereof. In another embodiment, the heterologous sequence encodes for any SstR agonist or an SstR antagonist. In another embodiment, the heterologous sequence encodes for a somatostatin analogue that is an intermediate SRIF/octapeptide analogue, octapeptide, or cyclohexapeptide. The use of Sst and its analogues in the stimulation of SstR is well known in the art, and is described, for example, in Weckbecker G et al, Nature Reviews Drug Discover 2:999-1017, (2003) and Weckbecker G et al, Endocrinology 143: 4123-4130 (2002), and references cited therein. Each analogue or homologue of somatostatin or of a related protein represents a separate embodiment of the present invention.

In another embodiment, the heterologous peptide is encoded for by the Sst nucleotide sequence set forth in SEQ ID No 1 (Example 1). In another embodiment, the inserted peptide is encoded for by an Sst nucleotide sequence or a homologue thereof such as that disclosed in NCBI's Entrez nucleotide database, having the Accession Number: BC032625, BC010770, CB067463, NM4001048, E16440, J00306, AX951322, E16436, E16420, H40660, W56163, or J00306.

In another embodiment, the heterologous peptide comprises sequence from a peptide or peptide hormone other than somatostatin. In one embodiment, the heterologous peptide is adrenalin, a calcitonin gene-related peptide, adrenomedullin, amylin, calcitonin, or a homologue thereof. In another embodiment, the heterologous peptide is angiotensin or a homologue thereof. In another embodiment, the heterologous peptide is a chemokine. In another embodiment, the heterologous peptide is a growth factor. In another embodiment, the heterologous peptide is a cytokine. In another embodiment, the heterologous peptide is vasopressin, oxytocin or a homologue thereof. In another embodiment, the heterologous peptide is insulin or a homologue thereof. In another embodiment, the heterologous peptide is an orexin. In another embodiment, the heterologous peptide is glial cell line-derived neurotrophic factor or a homologue thereof. In another embodiment, the heterologous peptide is gonadotrophin-releasing hormone or a homologue thereof. In another embodiment, the heterologous peptide is follicle-stimulating hormone, a gonadotrophin, or a homologue thereof. In another embodiment, the heterologous peptide is prolactin or a homologue thereof. In another embodiment, the heterologous peptide is vasoactive intestinal polypeptide (VIP), neurophysin, bombesin, or a homologue thereof. In another embodiment, the heterologous peptide is glial cell line-derived neurotrophic factor or a homologue thereof. In another embodiment, the heterologous peptide is neurotensin or a homologue thereof. In another embodiment, the heterologous peptide is an endothelin, a sarafotoxin, or a homologue thereof. In another embodiment, the heterologous peptide is a member of the CGRP peptide family. In another embodiment, the heterologous peptide is tachykinin, a tachykinin-like peptide, or a homologue thereof. In another embodiment, the heterologous peptide is orphanin FQ/nociceptin peptide or a homologue thereof. In another embodiment, the heterologous peptide is somatomedin or a homologue thereof. In another embodiment, the heterologous peptide is a pro-thyrotropin-releasing hormone-derived peptide or a homologue thereof. In another embodiment, the heterologous peptide is a natriuretic peptide or a homologue thereof. In another embodiment, the heterologous peptide is formyl peptide or a homologue thereof. In another embodiment, the heterologous peptide is peptide YY, pancreatic polypeptide, or a homologue thereof. In another embodiment, the heterologous peptide is a follitropin, lutropin, choriogonadotropin, or a homologue thereof. In another embodiment, the heterologous peptide is neutrophil peptide or a homologue thereof. In another embodiment, the heterologous peptide is a peptide growth factor such as, for example, platelet-derived growth factor (PDGF), fibroblast growth factor-2 (FGF-2), or connective tissue growth factor (CTGF). In another embodiment, the heterologous peptide is a bradykinin-related peptide or a homologue thereof. In another embodiment, the heterologous peptide is a member of the Adenylate Cyclase-Activating Polypeptide (PACAP)/Glucagon Superfamily. In another embodiment, the heterologous peptide is a chemotaxis factor such as, for example, mouse chemerin and human TIG, stromal cell derived factor-1 alpha (SDF-1a), SLC, MCP-1 or ELC, or a homologue thereof. In another embodiment, the heterologous peptide is a chemotaxis and differentiation factor such as, for example, protease activated receptor-1 (PAR-1) ligand, or protease activated receptor-2 (PAR-2) ligand, or Flt3 ligand, or a homologue thereof. In another embodiment, the heterologous peptide is any peptide or peptide hormone known in the art that interacts with a protein of interest. Each peptide represents a separate embodiment of the present invention.

In one embodiment, the heterologous peptide interacts directly with a cellular molecule. In another embodiment, the peptide interacts with an epitope tag or other sequence engineered into a cellular molecule. In another embodiment, the peptide interacts with a cellular molecule, e.g., via an antibody or other adaptor molecule.

A variety of epitopes is used to tag a protein, while retaining at least part of the biological activity of the unmodified protein. Such epitopes is naturally-occurring amino acid sequences found in nature, artificially constructed sequences, or modified natural sequences. Recently, a variety of artificial epitope sequences have been described that have been shown to be useful for tagging and detecting recombinant proteins. In one embodiment, an artificial epitope sequence with the eight amino acid FLAG marker peptide (Asp-Tyr-Lys-Asp-Asp-Asp-Asp-Lys) (SEQ ID No 19), has been useful for detection as well as affinity purification of recombinant proteins, with antibodies recognizing the epitope readily available (Kunz D et al, J. Biol. Chem. 267:9101-9106, 1992).

Additional artificial epitope tags include an improved FLAG tag having the sequence Asp-Tyr-Lys-Asp-Glu-Asp-Asp-Lys (SEQ ID No 20), a nine amino acid peptide sequence Ala-Trp-Arg-His-Pro-Gln-Phe-Gly-Gly (SEQ ID No 21) referred to as the “Strep tag” (Schmidt T G M et al, Prot. Engineering 6:109-122, 1993), poly-histidine sequences, e.g., a poly-His of six residues which is sufficient for binding to IMAC beads, an eleven amino acid sequence from human c-myc recognized by monoclonal antibody 9E10, or an epitope represented by the sequence Tyr-Pro-Tyr-Asp-Val-Pro-Asp-Tyr-Ala-Ile-Glu-Gly-Arg (SEQ ID No 22) derived from an influenza virus hemagglutinin (HA) subtype, recognized by the monoclonal antibody 12CA5. Also, the Glu-Glu-Phe sequence (SEQ ID No 23) recognized by the anti-alpha-tubulin monoclonal antibody YL1/2, has been used as an affinity tag for purification of recombinant proteins (Stammers D K et al. FEBS Lett. 283:298-302, 1991).

Another commonly used artificial epitope is a poly-His sequence having six histidine residues (His-His-His-His-His-His) (SEQ ID No 24). Naturally occurring epitopes include the eleven amino acid sequence from human c-myc recognized by the monoclonal antibody 9E10 (Glu-Gln-Lys-Leu-Leu-Ser-Glu-Glu-Asp-Leu-Asn) (SEQ ID No 25) (Manstein et al. (1995) Gene 162:129-134).

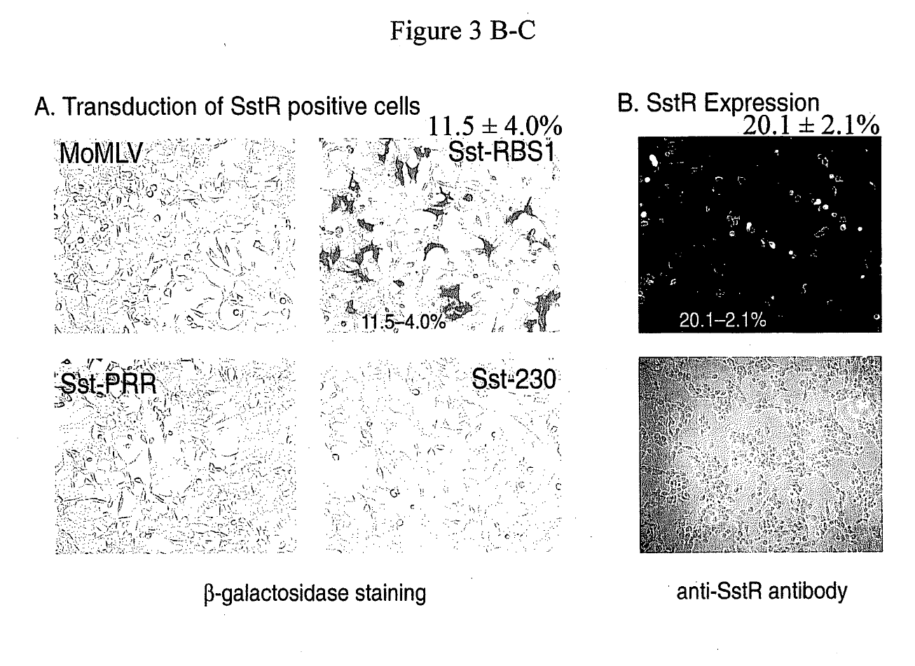

In one embodiment, the cellular molecule that interacts with the heterologous peptide is a somatostatin receptor (SstR) (FIG. 3). In another embodiment, the cellular molecule is the growth hormone secretagogue receptor, a receptor related to the SstR proteins (Weckbecker G et al, Nature Reviews Drug Discovery 2:999-1017, 2003). In another embodiment, the cellular molecule is a member of the Heptahelical receptor (HHR) family. HHRs are a family of receptors related to the SstR family (Patel R C et al, Proc Natl Acad Sci USA 99:3294-99, 2002). Each receptor or homologue or isoform thereof represents a separate embodiment of the present invention.

In another embodiment, the cellular molecule is any receptor for a peptide or peptide hormone that is known in the art. In one embodiment, the cellular molecule is a member of the opioid receptor family. In another embodiment, the cellular molecule is a member of the G-protein-coupled receptor family. In another embodiment, the cellular molecule is a member of the guanylyl cyclase-coupled receptor family. In another embodiment, the cellular molecule is a member of the Toll-like receptor family. In another embodiment, the cellular molecule is a member of the transmembrane tyrosine kinase receptor family. In another embodiment, the cellular molecule is a member of the transmembrane serine-threonine receptor family. In another embodiment, the cellular molecule is a member of the Ig receptor superfamily. In another embodiment, the cellular molecule is a member of the rhodopsin-like family. In another embodiment, the cellular molecule is a member of the transmitter-gated ion channel family. In another embodiment, the cellular molecule is a member of the cytokine receptor superfamily. In another embodiment, the cellular molecule is glycosylphosphatidylinositol (GPI)-anchored GDNF family receptor or a homologue thereof. In another embodiment, the cellular molecule is insulin receptor. IGF-1 receptor, or a homologue thereof. In another embodiment, the cellular molecule is receptor for an Adenylate Cyclase-Activating Polypeptide (PACAP)/Glucagon family member. In another embodiment, the cellular molecule is prepro-TRH160-169 (pST10) receptor or a homologue thereof. In another embodiment the cellular molecule is a phospholipase C (PLC)2-coupled receptor or a homologue thereof. In another embodiment, the cellular molecule is a receptor for a chemotaxis factor, such as, for example, chemerin receptor 23 (ChemR23), CXC chemokine receptor 4 (CXCR4), CC chemokine receptor 7 (CCR7), or CC chemokine receptor 5 (CCR5), or a homologue thereof. In another embodiment, the heterologous peptide is a chemotaxis and differentiation factor such as, for example, protease activated receptor-1 (PAR-1), or protease activated receptor-2 (PAR-2) ligand, or Flt3, or a homologue thereof. In another embodiment, the cellular molecule is a member of the integrin superfamily, such as, for example, alpha5beta7 integrin, or alpha3betaV integrin, or homologue thereof. In another embodiment, the cellular molecule is alpha-dystroglycan (a-DG), or homologure thereof. In another embodiment, the cellular molecule is an adrenergic or muscarinic receptor or a homologue thereof. In another embodiment, the cellular molecule is a steroidogenic factor or a homologue thereof. In another embodiment, the cellular molecule is a member of the IL-6 cytokine superfamily. In another embodiment, the cellular molecule is any protein known in the art that interacts specifically or preferentially with a peptide of interest. The use of peptide hormones and their receptors is well known in the art, and is described, for example, in the following review articles: Binder, E B et al, Pharmacol Rev 53: 453-486 (2001); Missale C et al, Physiol. Rev. 78: 189-225 (1998); Bowery, N G et al, Pharmiacol Rev 54: 247-264 (2002); Barnard, E A et al, Pharmacol Rev 50: 291-314 (1998); Poyner. D R et al, Pharmacol Rev 54: 733-246 (2002); and Mogil, J S et al, Pharmacol Rev 53: 381-415 (2001). Each peptide or receptor represents a separate embodiment of the present invention.

In one embodiment, the cellular molecule is any protein known in the art that is located on a surface of the target cell. In another embodiment, the cellular molecule is any protein known in the art that is located in a clathrin coated pit, caveloa, endocytic vesicle, or the like. Each cellular molecule represents a separate embodiment of the present invention.

In one embodiment, the cellular molecule occurs naturally in the target cell. In another embodiment, the target cell is engineered to comprise the cellular molecule. In one embodiment, the cellular molecule is a protein, glycoprotein, lipid, glycolipid, or any other molecule on the surface of the cell of interest. Each type of cellular molecule represents a separate embodiment of the present invention.

In another embodiment, the present invention provides a recombinant protein comprising the isolated polypeptide. In one embodiment, the recombinant protein comprises a sequence corresponding to or homologous to the sequence set forth in SEQ ID No 42 (Example 1).

In another embodiment of the present invention, the recombinant protein comprises an amino acid sequence from an MoMLV env protein or a homologue thereof, such as the sequence:

MARSTLSKPLKNKVNPRGPLIPLILLMLRGVSTASPGSSPHQVYNITWEV
TNGDRETVWATSGNHPLWTWWPDLTPDLCMLAHHGPSYWGLEYQSPFSSP
PGPPCCSGGSSPGCSRDCEEPLTSLTPRCNTAWNRLKLDQTTHKSNEGFY
VCPGPHRPRESKSCGGPDSFYCAYWGCETTGRAYWKPSSSWDFITVNNNL
TSDQAVQVCKDNKWCNPLVIRFTDAGRRVTSWTTGHYWGLRLYVSGQDPG
LTFGIRLRYQNLGPRVPIGPNPVLADQQPLSKPKPVKSPSVTKPPSGTPL
SPTQLPPAGTENRLLNLVDGAYQALNLTSPDKTQECWLCLVAGPPYYEGV
AVLGTYSNHTSAPANCSVASQHKLTLSEVTGQGLCIGAVPKTHQALCNTT
QTSSRGSYYLVAPTGTMWACSTGLTPCISTTILNLTTDYCVLVELWPRVT
YHSPSYVYGLFERSNRHKREPVSLTLALLLGGLTMGGIAAGIGTGTTALM
ATQQFQQLQAAVQDDLREVEKSISNLEKSLTSLSEVVLQNRRGLDLLFLK
EGGLCAALKEECCFYADHTGLVRDSMAKLRERLNQRQKLFESTQGWFEGL
FNRSPWFTTLISTIMGPLIVLLMILLFGPCILNRLVQFVKDRISVVQALV
LTQQYHQLKPIEYEP (SEQ ID No 40, Gen Bank Accession
# J02255).

in another embodiment, the recombinant protein comprises an amino acid sequence corresponding to or homologous to an MoMLV env amino acid sequence such as that disclosed in NCBI's Entrez protein database, having the Accession Number: P03385, NP_{—}057935, AAL69911, VCVWEM, AAB32464, AAB32-463, AAC82567, AAB59943, AAB59942, 0711245A, or AAA46517, or a homologue thereof. In another embodiment, the recombinant comprises a sequence from an env protein of other retrovirus or lentivirus. In another embodiment, the recombinant protein comprises a sequence from a protein other than a retroviral or lentiviral env protein.

In another embodiment, the recombinant protein comprises a cytoplasmic tail from a protein other than MoMLV env protein. In one embodiment, the cytoplasmic tail is from an env protein of a retrovirus or lentivirus other than MoMLV. In another embodiment, the recombinant protein comprises a cytoplasmic tail from a protein other than MMTV env protein. In another embodiment, the cytoplasmic tail is from a protein other than an env protein. A cytoplasmic tail, in one embodiment, refers to a portion of a transmembrane protein that is on the cytoplasmic side of a cellular membrane, or a fragment thereof.

In another embodiment, the cytoplasmic tail is from MoMLV env protein. Replacing the cytoplasmic tail of MMTV-Sst-RBM env protein with this cytoplasmic tail increased the infectivity of pseudotyped viruses comprising the mutant protein, MMTV-Sst-RBM-MoMLV-cyt.

In another embodiment, addition of a cytoplasmic tail from a protein other than MoMLV env protein increases infectivity of a virus comprising the mutant protein. In another embodiment, a cytoplasmic tail from a protein other than MoMLV env protein increases production of a virus comprising the mutant protein. In another embodiment, the cytoplasmic tail increases incorporation of the mutant protein into a virus. In another embodiment, the cytoplasmic tail increases incorporation of a nucleic acid into a virus. In another embodiment, the cytoplasmic tail alters specificity of incorporation of nucleic acid into a virus. In another embodiment, the cytoplasmic tail alters another desired characteristic of the mutant protein or a recombinant virus comprising same. Each cytoplasmic tail represents a separate embodiment of the present invention.

In another embodiment, the present invention provides a method for increasing the infectivity of an env protein, comprising pseudotyping the cytoplasmic tail of the env protein (Example 13). In another embodiment, sequence from the cytoplasmic tail of the different env protein replaces all or part of the sequence encoding the cytoplasmic tail of the env protein of the present invention. In another embodiment, sequence from the cytoplasmic tail of the different env protein is inserted into the gene encoding the env protein of the present invention. In one embodiment, the original env protein is a mild-type env protein. In another embodiment, the original env protein is a recombinant env protein. In another embodiment, the original env protein is a recombinant env protein of the present invention. Each possibility represents a separate embodiment of the present invention.

In another embodiment of the present invention, the recombinant protein comprises an amino acid sequence from an MMTV en protein or a homologue thereof, such as the sequence:

(SEQ ID No 10)

MPKHQSGSPIGSSDLLLSGKKQRPHLALRRKRRREMRKIINRKVRRMNLA
PIKEKTAWQHLQALIFEAEEVLKTSQTPQTSLTLFLALLSVLGPPPVTGE
SYWAYLPKPPWHPVGWGNTDPIRVLTNQTIYLGGSPDFHGFRNMSGNVHF
EGKSDTLPICFSFSFSTPTGCFQVDKQVFLSDTPTVDNNKPGGKGDKRRM
WELWLTTLGNSGANTKLVPIKKKLPPKYPHCQIAFKKDAFWEQDESAPPR
WLPCAFPDQGVSFSPKGALGLLWDFSLPSPSVDQSDQIKSKKDLFGNYTP
PVNKEVFIRWYEAGWVEPTWFWENSPKDPNDRDFTALVPHTELFRLVAAS
RYLILKRPGFQEHDMIPTSACVTYPHAILLGLPQLIDIEKRGSTFHISCS
SCRLTNCLDSSAYDYAAIIVKRPPYVLLPVDIGDEPWFDDSAIQTFRYAT
DLIRAKRFVAAIILGISALIAIITSFAVATTALVKEMQTATFVNNLHRNV
TLALSEQRIIDLKLEARLNALEEVVLELGQDVANLKTRMSTRCHANYDFI
CVTPLPYNASESWERTKAHLLGIWNDNELSYNIQELTNLISDMSKQFIID
TVDLSGLAQSFANGVKALNPLDWTQYFIFIGVGALLLVIVLMIFPIVFQC
LAKSLDQVQSDLNVLLLKKKKGGNAAPAAEMVELPRVSYT.

In another embodiment, the recombinant protein comprises an amino acid sequence corresponding to or homologous to an MMTV env amino acid sequence such as that disclosed in NCBI's Entrez protein database, having the Accession Number: S26388, VCMVMM, P03374, P10259, AAC82558, CAA25954, CAA25955, BAA03768, AAF31475, AAF31470, AAF64164 or AAC24861, or a homologue thereof. In another embodiment, the recombinant protein comprises a sequence from an MMTV env precursor disclosed herein. In another embodiment, the recombinant protein comprises a sequence from an en, protein of other retrovirus or lentivirus, or a precursor or homologue thereof. In another embodiment, the recombinant protein comprises a sequence from a protein other than a retroviral or lentiviral env protein.

In one embodiment, the mutant or variant RBM comprises a mutation in an RBM of an env gene. In another embodiment, the mutated RBM comprises a mutation at a site encoding an amino acid within 5 amino acids of an RBM of an env protein; or, in another embodiment, within 10 amino acids of the RBM; or, in another embodiment, within 20 amino acids of the RBM; or, in another embodiment, within 30 amino acids of the RBM; or, in another embodiment, within 40 amino acids of the RBM; or, in another embodiment, within 50 amino acids of the RBM. Each location represents a separate embodiment of the present invention.

In another embodiment, the present invention provides a recombinant viral particle, cell or packaging cell line comprising a recombinant protein of the invention. In another embodiment, the recombinant protein comprised in the recombinant viral particle alters the tropism of the viral particle. In another embodiment, the recombinant protein does not alter the tropism of the viral particle.

It will be appreciated by those skilled in the art that, in one embodiment, a gene encoding a recombinant protein of the present invention comprises additional mutations other than those affecting the RBM. In another embodiment, a nucleic acid molecule present in a recombinant viral particle or the present invention comprises additional mutations other than a mutation in a gene encoding for the recombinant protein of the invention. In another embodiment, for example, the nucleic acid molecule comprises a mutation in a virulence factor. In another embodiment, the nucleic acid molecule comprises a mutation in a non-coding sequence that affects the expression of an encoded protein. In another embodiment, the nucleic acid molecule comprises any other mutation other than a mutation in a gene encoding for the recombinant protein of the invention, whether the mutation was intentionally added or arose naturally. Each mutation or combination thereof represents a separate embodiment of the present invention.

In another embodiment, the present invention provides a recombinant viral particle, comprising: a. a mutated MMTV or MoMLV env protein comprising a mutated RBM; and b. a heterologous nucleic acid of interest. In this embodiment, the recombinant viral particle is used to deliver the heterologous nucleic acid of interest to a cell. Any embodiment listed herein for; the mutated RBM of an MMTV env protein is used in any recombinant viral particle of the present invention, and is to be considered a separate embodiment of the invention.

In another embodiment, a nucleic acid of interest of any method of the present invention encodes a biologically functional protein, i.e. a polypeptide or protein that affects the cellular mechanism of the target cell. In one embodiment, the biologically functional protein is a protein that is beneficial for normal growth of the cell or for maintaining the health of an animal or human. The biologically functional protein is, in another embodiment, a protein that improves the health of a animal or human by either supplying a missing protein, by providing increased quantities of a protein which is deficient in the animal or human or by providing a protein which inhibits or counteracts an undesired molecule which is present in the animal or human. The biologically functional protein is, in another embodiment, a protein that is useful for investigative studies directed to developing new gene therapies or studying cellular mechanisms.

The biologically functional protein is, in another embodiment, be a protein that is beneficial for normal growth or repair of the human body. The biologically functional protein is, in another embodiment, useful in fighting diseases such as cancer, atherosclerosis, sickle-cell anemia and the thalassemias. Examples of such biologically functional proteins are hemoglobin (alpha-, beta-, or gamma-globin), hematopoietic growth factors such as granulocyte-macrophage colony stimulating factor (GM-CSF), macrophage colony stimulating factor (M-CSF), granulocyte colony stimulating factor (G-CSF) and erythropoietin (EPO). In another embodiment, the biologically functional protein is tumor necrosis factor (TNF), a molecule that can be used to treat cancer or another condition involving a cancer cell or neoplastic cell. In another embodiment, the biologically functional protein is a tumor suppressor such as, for example, p53 or retinoblastoma (RB). In another embodiment, the biologically functional protein is one of various cytokines such as, for example, mast cell growth factor (MGS) and interleukins 1-11. The biologically functional protein is, in another embodiment, a selectable marker for antibiotic resistance such as neomycin resistance. In another embodiment, the biologically functional protein is a different type of selectable markers such as adenine phosphoribosyl transferase (APRT) in APRT-deficient cells, or the firefly luciferase gene. In another embodiment, the biologically functional protein is any protein known in the art, the presence of which is desired in a target cell. For many biologically functional proteins, DNA encoding the protein is commercially available. Each protein represents a separate embodiment of the present invention.

In another embodiment, the nucleic acid of interest can encode a recombinant protein comprising various domains and functions from a variety of sources. In another embodiment, the nucleic acid of interest can comprise a mutation of any sort in a wild-type gene sequence, thus encoding a mutated version of a biologically functional protein.

In another embodiment the nucleic acid of interest is transcribed into an RNA molecule that is able to hybridize to an mRNA or DNA of interest. Such an RNA molecule is hereinafter referred to as antisense RNA, and has, in one embodiment, utility in preventing or limiting the expression of overproduced, defective, or otherwise undesirable molecules. In one embodiment, the antisense RNA prevents or limits transcription of the mRNA of interest or an mRNA encoded by the DNA of interest. In another embodiment, the antisense RNA prevents translation of the mRNA of interest. In one embodiment, the mRNA or DNA of interest is a region of a gene that encodes for a polypeptide. In another embodiment, the mRNA or DNA of interest is non-coding region of a gene, such as, or example, a promoter or enhancer region. Each nucleic acid of interest represents a separate embodiment of the present invention.

In another embodiment, the protein of interest encoded for by the heterologous nucleic acid of interest is therapeutic. In another embodiment, the protein of interest is immunogenic. Each protein or interest represents a separate embodiment of the present invention.

In another embodiment, the nucleic acid of interest introduced by the recombinant viral particle is a non-coding regulatory sequence that does not encode an mRNA or protein product (e.g., promoter sequence, polyadenylation sequence, termination sequence, enhancer sequence, etc.). In another embodiment, the nucleotide sequence is a gene encoding a vaccine or antigen. In another embodiment, the heterologous nucleic acid of interest is a ribozyme, antisense gene, or other non-coding sequence. In another embodiment, the heterologous nucleic acid of interest is any embodiment of a nucleic acid disclosed herein, for which its introduction into a cell has utility. Each nucleic acid represents a separate embodiment of the present invention.

In another embodiment, the recombinant viral particle further comprises a retroviral genome or a lentiviral genome. The retroviral genome or lentiviral genome is, in one embodiment, a genome any retrovirus or lentivirus known in the art other than MMTV. In this embodiment, the virus from which the genome is derived is said to be “pseudotyped” with the recombinant protein of the invention, a term denoting the engineering of a virus to comprise a protein derived from a different virus. In one embodiment, the gene for the MMTV env protein is provided either in cis, e.g., on the same nucleic acid molecule, as the retroviral or lentiviral genome, or in trans, e.g., on a separate nucleic acid molecule. Methods for pseudotyping viruses are well known in the art, and are described, for example, in Steele T A, Proceedings of the Society for Experimental Biology and Medicine 223:118-127 (2000), in U.S. Pat. No. 6,448,390, and in references cited therein. Each method represents a separate embodiment of the present invention.

Any embodiment listed herein for an isolated nucleic acid, isolated polypeptide, or recombinant viral particle is used in any method of the invention, and is to be considered a separate embodiment of the invention.

Similarly, any embodiment for an RBM of the present invention is utilized in any method of the present invention. In another embodiment, an RBM homologous to an RBM of the present invention is utilized in a method of the present invention. In another embodiment, a non-RBM peptide sequence that is structurally similar to an RBM of the present invention is utilized in a method of the present invention. Each tripe of RBM represents a separate embodiment of the present invention.

As used herein, the term “contacting”, “contact” or “contacted” when in reference to a cell indicates, in one embodiment, both direct and indirect exposure of the cell to a nucleic acid, peptide, protein, vector, compound or composition of the invention. It is envisaged that, in another embodiment, that supply to the cell is indirect, such as via provision in a culture medium that surrounds the cell, or via parenteral administration in a body of a subject in need, whereby the agent ultimately contacts a cell via peripheral circulation (for further detail see, for example, Methods in Enzymology Vol. 1-317, Rubin and Dennis, eds, (1955-2003) and Current Protocols in Molecular Biology, Ausubel, et al. eds (1998), Molecular Cloning: A Laboratory Manual, Sambrook and Russell, eds., (2001), or other standard laboratory manuals). It is to be understood that any direct means or indirect means of intracellular access of a viral particle, nucleic acid, vector, or peptide of the invention represents an embodiment thereof.

In another embodiment, the virus whose genome is comprised in the recombinant viral particle is a lentivirus or retrovirus. In one embodiment, the virus is an integrating virus. In another embodiment, the virus is a non-integrating virus. In another embodiment, the virus is an MLV virus. In another embodiment, the MLV virus is ecotropic. In another embodiment, the virus is Moloney MLV (MoMLV). MoMLV is a virus that belongs to the ecotropic class of MLV viruses, those MLV viruses that can replicate in murine cells only. The present invention shows that MoMLV virus pseudotyped with MMTV env protein can be successfully utilized for delivering a nucleic acid of interest to a target cell (Example 1). In another embodiment, the virus is any virus for which introduction into a cell is deemed desirable for any reason.

In another embodiment, the genome is comprised in the recombinant viral particle is a genome of a virus other than the virus from which the mutated env protein was derived.

In another embodiment, the genome comprised in the recombinant viral particle comprises a disruption or deletion of a gene in the genome that encodes an env protein, the encoded protein herein referred to as the “endogenous env protein”. The disruption or deletion decreases, in one embodiment, the amount of the endogenous env protein in the viral particle. In another embodiment, the disruption or deletion eliminates the endogenous env protein in the viral particle.

In another embodiment, decreasing or eliminating the endogenous env protein in the viral particle decreases or eliminates entry or infection of a cell expressing a viral receptor for the endogenous env protein. In another embodiment, decreasing or eliminating the protein narrows the tropism of the recombinant viral particle. In another embodiment, decreasing or eliminating the protein increases the efficacy of the recombinant viral particle as a gene therapy vector by reducing or eliminating infection of cells other than the cells targeted for gene therapy. Each possibility represents a separate embodiment of the present invention.

In another embodiment, the present invention provides a method for delivering a nucleic acid of interest to a target cell, comprising contacting the target cell with a recombinant viral particle or liposome comprising (a) nucleic acid of interest or compound of interest; and (b) a mutated retroviral or lentiviral env protein comprising a heterologous peptide, whereby the heterologous peptide mediates uptake of the recombinant viral particle or liposome via a cellular target molecule, thereby delivering a nucleic acid of interest to a target cell.

In one embodiment, the heterologous peptide inserted into mutated env proteins fo the present invention functions via fusion of a membrane of the target cell with a membrane of the recombinant viral particle. In one embodiment, the fusion is pH dependent, as is the case with MMTV. In another embodiment, the fusion is pH independent. Each possibility represents a separate embodiment of the present invention.

Methods of preparing a liposome comprising a membrane-bound or transmembrane protein are well known in the art, and are described, for example, in Bach M, et al, Protein Eng. 2003 December; 16(12):1107-13; Giovagnoli S et al, AAPS Pharm Sci Tech. 2004 Dec. 31; 4(4):E69; Kakudo T et al, Biochemistry. 2004 May 18; 43(19):5618-28: Gabizon A, et al, Adv Drug Deliv Rev. 2004 Apr. 29; 56(8):1177-92; Lopez-Barcons L A, et al, J Biomed Mater Res. 2004 Apr. 1; 69A(1):155-63; Smith S A, et al, J Thromb Haemost. 2004 July; 2(7):1155-62; Rigaud J L et al, Biochemistry. 1988 Apr. 19; 27(8):677-88; and Levy D et al, Biochim Biophys Acta 1990 Jun. 27; 1025(2):179-90.

In one embodiment, any embodiment of a mutated RBM of the present invention is utilized in any method of the present invention. In another embodiment, a mutated RBM homologous to a mutated RBM of the present invention is utilized in a method of the present invention. In another embodiment, a non-RBM peptide sequence that is structurally similar to a mutated RBM of the present invention is utilized in a method of the present invention. Each type of RBM represents a separate embodiment of the present invention.

In one embodiment, the target cell of any method of the present invention is a cancer cell or neoplastic cell. “Neoplastic cell” refers, in one embodiment, to a cell whose normal growth control mechanisms are disrupted (typically by accumulated genetic mutations), thereby providing potential for uncontrolled proliferation. Thus, “neoplastic cell” can include, in one embodiment, both dividing and non-dividing cells. For purposes of the invention, neoplastic cells include cells of tumors, neoplasms, carcinomas, sarcomas, leukemias, lymphomas, and the like. In another embodiment, “neoplastic cells” can include central nervous system tumors, especially brain tumors. These include glioblastomas, astrocytomas, oligodendrogliomas, meningiomas, neurofibromas, ependymomas, Schwannomas, neurofibrosarcomas, etc. In another embodiment, “neoplastic cells” can include either benign or malignant neoplastic cells. In another embodiment, “neoplastic cells” can include any other type of cancer known in the art. It is desirable, in one embodiment, to utilize a compound toxic to the cancer cell or neoplastic cell as the compound of interest in order to weaken or eliminate cancer cells or neoplastic cells.

In one embodiment, the target cell is an infected cell. In another embodiment, the target cell is a pathogenic cell. In another embodiment, the target cell mediates autoimmunity or another disease state. In another embodiment, the target cell is deficient or lacking in the expression of a cellular gene necessary for a physiological function. The nucleic acid of interest restores, in another embodiment, the physiological function by replacing or compensating for the lack of expression of the cellular gene. Each target cell represents a separate embodiment of the present invention.

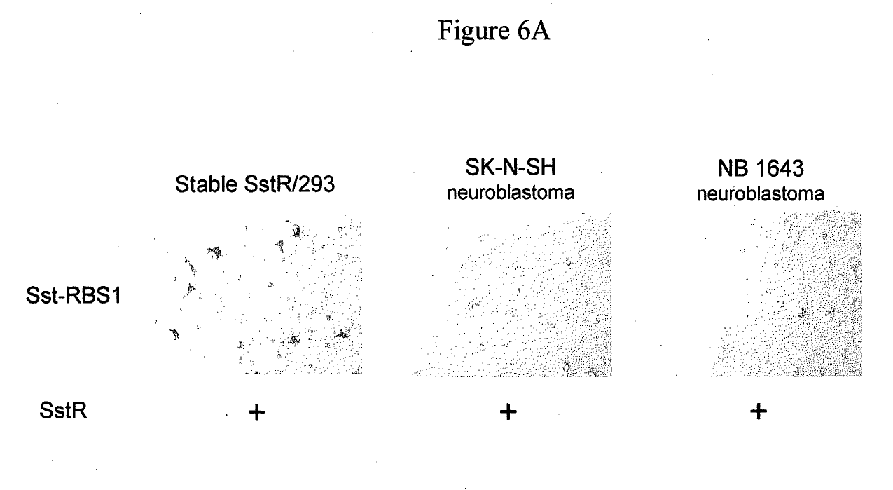

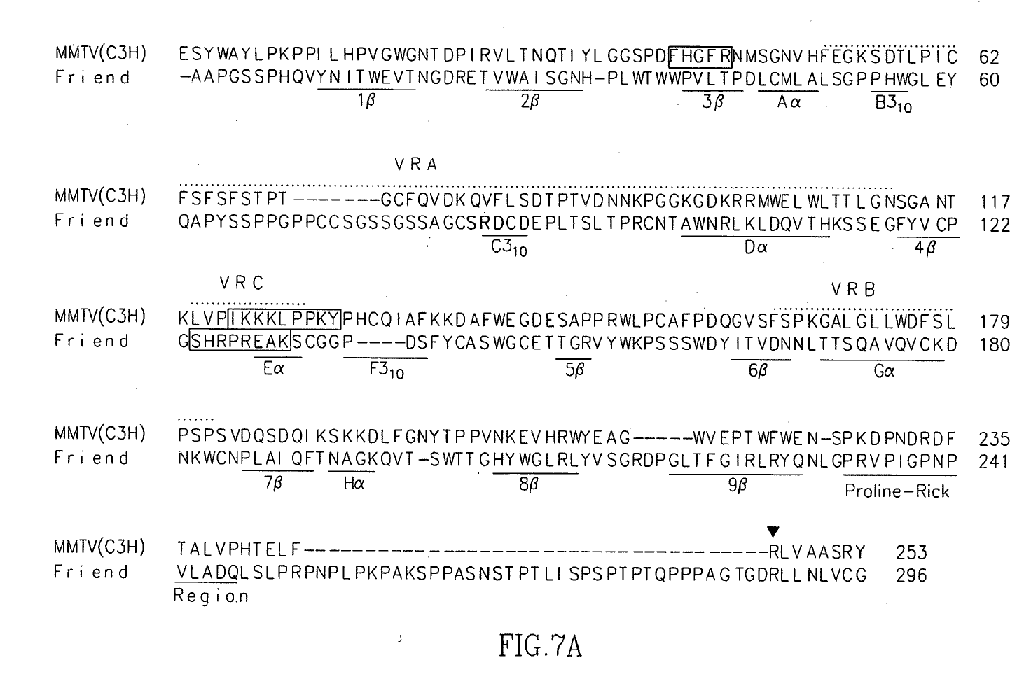

In one embodiment, the mutated retroviral or lentiviral env protein that is comprised in the recombinant viral particle or liposome is derived from a retrovirus or lentivirus resistant to lysosomal degradation. It was shown in the present invention that recombinant env proteins derived from MoMLV, which is susceptible to lysosomal degradation, were unable to infect cells in which cathepsins were activated (FIG. 6), showing that recombinant viral particles is degraded by lysosomal proteases. This finding demonstrated that utilization of an env protein that is resistant to lysosomal degradation can overcome the problem of degradation of a recombinant viral particle or liposome used for a gene delivery application.

Cathepsins are, in one embodiment, proteases that belong to the papain superfamily of lysosomal cysteine proteases Cathepsinis participate in many cellular functions. These proteases are localized in lysosomes and other intracellular compartments, and have maximal activity at acidic pH.

In one embodiment, a virus particle or protein is considered to be resistant to lysosomal degradation if it is able to remain essentially intact in a lysosome long enough to enter the cytoplasm of the target cell after being internalized in the lysosome. In another embodiment, “resistant to lysosomal degradation” refers to an ability to remain functional or replication-competent in a lysosome long enough to enter the cytoplasm of the target cell after being internalized in the lysosome. In another embodiment, the term refers to an ability to remain essentially intact for several minutes in a lysosome. In another embodiment, the term refers to an ability to remain essentially intact for several seconds in a lysosome. In another embodiment, the term refers to an ability to remain functional or replication-competent for several minutes in a lysosome. In another embodiment, the term refers to an ability to remain functional or replication-competent for several minutes in a lysosome. Functional is defined, in one embodiment, as able to introduce into the host cell a nucleic acid that is able to be copied, transcribed, or translated.

In one embodiment, the mutation of the RBM increases fusion of the recombinant viral particle or liposome with the target cell. In the present invention, insertion of an Sst peptide conferred upon the env proteins of MoMLV and MMTV the ability to mediate infection of cells expressing SstR (FIG. 3). In one embodiment, increasing fusion of the recombinant viral particle or liposome with the target cell reduces the dose of viral particles required to exert a biological effect desired for a medical (e.g., gene therapy) or scientific application.

In one embodiment, the increased fusion is mediated by the cellular molecule that interacts with the mutant or variant env protein. In another embodiment, the cellular molecule that interacts with the mutated env gene does not mediate fusion, but rather brings the viral particle into proximity of a different cellular molecule that mediates fusion.

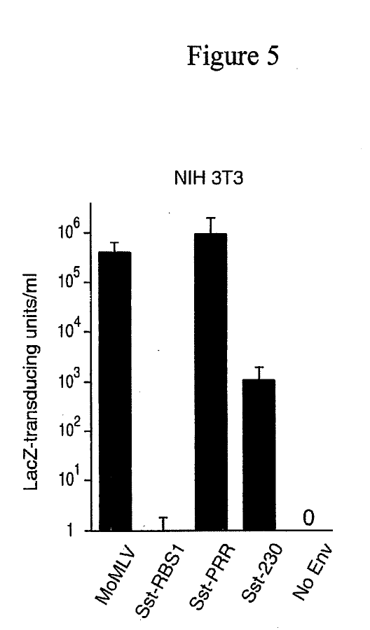

In another embodiment, deletion or substitution of all or part of the RBM diminishes or abrogates interaction or fusion with a cell other than the target cell. In another embodiment, insertion of a heterologous sequence into the RBM diminishes or abrogates interaction or fusion with a cell other than the target cell. The present invention has shown that substitution of a heterologous sequence for the RBM of MoMLV env protein eliminated its ability to enter cells via the natural MoMLV receptor (FIG. 5). Diminishing or abrogating interaction or fusion with a cell other than die target cell increases, in one embodiment, the number of viral particles that have access to the target cell. In another embodiment, diminishing or abrogating this interaction or fusion increases the efficacy of a viral particle used for gene therapy of another medical or research purpose. In another embodiment, diminishing or abrogating this interaction or fusion decreases the dose of a gene therapy vehicle or other viral particle used for medical or research purposes required to exert a desired biological effect. Each possibility represents a separate embodiment of the present invention.

In another embodiment, the mutant or variant retroviral or lentiviral env protein diminishes or abrogates interaction of the recombinant viral particle or liposome with a cellular molecule other than the cellular molecule utilized for uptake of the recombinant viral particle. The present invention shows that substitution of a heterologous sequence for the RBM of MoMLV env protein abrogates its interaction with the protein MCAT-1, the viral receptor for MoMLV.

In another embodiment, the present invention provides a method of treating or preventing a disease or disorder comprising a pathogenic cell that comprises a target cellular molecule, comprising contacting the pathogenic cell with a recombinant viral particle or liposome comprising a mutated retroviral or lentiviral env protein, wherein the retroviral or lentiviral env protein comprises an insertion of a peptide that interacts with the target cellular molecule, and whereby the target cellular molecule mediates entry or infection of the pathogenic cell by the recombinant viral particle, thereby treating or preventing a disease or disorder.

In one embodiment, the recombinant viral particle or liposome treats or prevents the disease or disorder by killing the pathogenic cell. In another embodiment, the recombinant viral particle or, liposome treats or prevents the disease or disorder by affecting the metabolism, growth, or any other characteristic of the pathogenic cell.

In one embodiment, the disease or disorder is caused by an overabundance or excessive proliferation of the pathogenic cell. In another embodiment, the pathogenic cell causes the disease or disorder by performing a pathogenic activity.

In other embodiments, the peptide inserted into the env protein is Sst or a related protein or homologue thereof. In this embodiment, the disease or disorder is any disease in which cells expressing SstR play a significant role, e.g., a disease or disorder for which and/or Sst analogues have been utilized as a treatment. Sst and or an Sst analogue has been used to treat the following diseases and disorders: diabetes type I and II; hypetsecretory tumors, such as growth hormone-secreting pituitary adenomas, gastrinomas, insulinomas, glucagonomas and vipomas; and gastrointestinal disorders, including gastric ulcers, pancreatitis, complications due to pancreatic surgery, pancreatic fistulae, acromegaly, diabetes type I and II; hypersecretory tumours, such as growth hormone-secreting pituitary adenomas, gastrinomas, insulinomas, glucagonomas and vipomas; and gastrointestinal disorders, acromegaly, gastroenteropancreatic tumours, chemotherapy-induced diarrhea, glucagonomas, insulinomas, carcinoid tumours, impaired secretion of growth hormone, gastritis, haemorrhagic pancreatitis, tissue damage caused by toxic agents, Cushing's disease, tumors of the thyroid, breast, prostate, gastrointestinal tract, colon, or pancreas, on small-cell lung cancer, neuro-endocrine tumors, malignant lymphoma, Graves' ophthalmopathy, diabetic retinopathy, diabetic nephropathy, various central nervous system and peripheral nervous system diseases, chronic pain, restenosis following angioplasty, graft vessel remodeling following organ transplantation, rheumatoid arthritis, inflammatory bowel disease, psoriasis, Graves' disease, multiple sclerosis, another immune-driven inflammatory disorder (Weckbecker G et al, Nature Reviews Drug Discovery 2:999-1017, 2003).

In other embodiments, the disease or disorder involves the secretion of growth hormone, prolactin, calcitonin, adrenocorticotropin, glucagon, insulin, interferon, gastric acid, glucagon-like peptide-1, amylase, ghrelin, gastric acid, bile, gastrin, secretin, 5-HT, dopamine, or any hormone known in the art whose secretion is modulated by an Sst. Sst analogues have been shove to modulate the secretion of these hormones, demonstrating that a significant number of cells that produce these hormones express a SstR. In this embodiment, secretion of the hormone of interest is modulated by targeting the recombinant viral particle or liposome to a cell secreting the hormone via the mutated retroviral or lentiviral env protein.

In another embodiment, the disease or disorder is any disease or disorder known in the art to involve a cell that expresses SstR or a related protein or homologue thereof. Each of the above diseases represents a separate embodiment of the present invention.

Different Sst analogues are available, and many of these exhibit differential affinities for different SstR's, enabling the targeting of a cell type expressing one or more particular SstR's by choosing one or more analogues specific to the SstR(s) expressed on the cell type. The use of Sst analogues is well known in the art, and is described, for example, in Weckbecker G et al, Nature Reviews Drug Discovery 2:999-1017, (2003). Each method for the use of Sst analogues represents a separate embodiment of the present invention.

In another embodiment, the cellular molecule that mediates uptake of the recombinant viral particle or liposome is routed to a cellular, compartment. In one embodiment, the cellular compartment has an acidic pH. In another embodiment, the cellular compartment does not have an acidic pH. In one embodiment, the cellular compartment is a lysosome. In another embodiment, the cellular compartment is any compartment known in the art with an acidic pH, e.g. a vacuole, endosome, or the like. Each type of compartment represents a separate embodiment of the present invention.

In one embodiment, routing of the cell molecule occurs after the cellular molecule interacts with the inserted peptide. In another embodiment, the routing occurs prior to interaction between the cellular molecule and the inserted peptide.

In one embodiment, routing refers to the movement of a molecule to a different location within the cell. In one embodiment, the movement is active. In another embodiment, the movement is passive. In another embodiment, the movement is mediated by diffusion or another process. In another embodiment, the movement is reversible or temporary. In another embodiment, the movement is irreversible or permanent. In another embodiment, routing refers to the uptake or internalization of a molecule into an intracellular vesicle.

In another embodiment, the present invention provides a method for delivering a compound of interest to a target cell, comprising contacting the target cell with a recombinant viral particle or liposome comprising: a. the compound of interest; and b. a mutant or variant retroviral or lentiviral env protein, comprising a mutation in a nucleic acid sequence encoding for an RBM of the mutant or variant retroviral or lentiviral env protein, whereby the mutant or variant RBM confers uptake of the recombinant viral particle or liposome via a cellular molecule, thereby delivering a compound of interest to a target cell.

In one embodiment, the compound of interest is cytotoxic. It is desirable, in one embodiment, to deliver a cytotoxic compound to a cancer cell or neoplastic cell such as a cancer cell or tumor cell, to an infected cell, or to a pathogenic cell. In another embodiment, the compound of interest is therapeutic. It is desirable, in one embodiment, to deliver a therapeutic compound to a cell that is failing to perform a beneficial function. In another embodiment, it is desirable to deliver a therapeutic compound to prevent, ameliorate or treat cell death by necrosis or apoptosis. Each method represents a separate embodiment of the present invention.

In one embodiment, the compound of interest is a drug or pharmaceutical agent. In another embodiment, the compound of interest is a wild type protein. In another embodiment, the compound of interest is a recombinant protein. Each type of compound of interest represents a separate embodiment of the present invention.

In another embodiment, the present invention provides a method of delivering a compound of interest to an acidified compartment of a target cell, comprising: a. chemically attaching the compound of interest to a mutant or variant MMTV env protein that is directed to the acidified compartment to form a mutant or variant MMTV env protein-compound complex; and b. contacting the target cell with the mutant or variant MMTV env protein-compound complex. In this method, the mutant or variant MMTV env protein is directed to the acidified compartment via interaction with a surface molecule of the target cell that is itself routed to the acidified compartment, thereby delivering a compound of interest to an acidified compartment of a target cell.

In one embodiment, the interaction with a surface molecule is mediated by a peptide inserted into the MMTV env protein. In one embodiment, the peptide comprises sequence from Sst. It is shown in the present invention that insertion of an Sst peptide into an env protein directs the env protein to a lysosomal compartment of a cell (Example 4). In another embodiment, the interaction of the mutated MMTV env protein with a surface molecule is mediated by any method of the present invention whereby a recombinant protein of the present invention interacts with a cellular molecule.

In another embodiment, the present invention provides a method of delivering a compound of interest to an acidified compartment of a target cell, comprising: a. chemically attaching the compound of interest to a recombinant protein to form a recombinant protein-compound complex, whereby the recombinant protein comprises an insertion of a heterologous peptide that results in routing of the recombinant protein to the acidified compartment; and b. contacting the target cell with the recombinant protein-compound complex. In this method, the recombinant protein is directed to the acidified compartment via interaction with a surface molecule of the target cell that is itself routed to the acidified compartment, thereby delivering a compound of interest to an acidified compartment of a target cell.

In one embodiment, the target cell is infected with a pathogen that resides in the acidified compartment. It is desirable, in one embodiment, to utilize a compound toxic to the pathogen as the compound of interest in order to combat the infection.

In another embodiment, the target cell exhibits a disease or disorder involving a lysosome, such as, for example, a lysosomal storage disease. In another embodiment, the disease is one or more of the following diseases: Fabry Disease, Farber Disease, Gaucher's Disease, GM1-Gangliosidosis, Krabbe Disease, Metachromaticleucodystrophy, Niemann-Pick Disease types A and B, Sandlhoff Disease, Tay Sachs Disease, Hurler Syndrome, Scheie Syndrome, Hunter Syndrome, Sanfilippo Syndrome, Morquio Syndrome, Maroteaux-Lamy Syndrome, Sly Sydrome, Pompe Disease, Aspartylglucosaminuria, Fucosidosis, Mannosidosis, Schindler Disease, Sialidosis, Galactosialidosis, Mucolipidosis types II and III, Multiple sulphatase deficiency, Pseudo-Hurler dystrophy, I-Cell disease, Niemann-Pick Disease type C1 & C2, Wolman Disease, Cystinosis, Infantile Sialic Acid Storage Disease, Salla Disease, Pycnodysostosis, Batten Disease, Ceroid Lipofuscinosis, or any other lysosomal disease known in the art. Each of these diseases represents a separate embodiment of the present invention.

In one embodiment, the target cell requires delivery of a therapeutic compound to the acidified compartment in order to enhance or improve its viability. In another embodiment, delivery of the therapeutic compound treats, ameliorates or prevents apoptosis or necrosis of the target cell.

In another embodiment, the activity of the compound of interest is enhanced or increased by acidic pH or by an enzyme in the acidified compartment. Such a compound exhibits, in one embodiment, reduced toxicity to cells other than the target cells. In another embodiment, the compound exhibits reduced systemic toxicity. In another embodiment, the compound exhibits increased toxicity for a cancer cell, a neoplastic cell, a diseased cell, or an infected cell. Increased toxicity occurs, in one embodiment, if the surface molecule is routed to the acidic compartment more efficiently in the target cell than in a healthy cell. It has been shown in the present invention that cancer cells efficiently deliver viral particles that interact with SstR to the lysosome (Example 4).

In another embodiment, increased toxicity occurs if the cancer, disease, or infection alters the pH of an acidic compartment of the cell. In another embodiment, increased toxicity occurs if the cancer, disease, or infection alters the expression or activity of an enzyme in the acidic compartment. Cathepsins have been shorten by the present invention to be activated in cancer cells (FIG. 6).

In another embodiment, the present invention provides a method of conferring upon a protein of interest an affinity for a TfR, comprising engineering the protein of interest to comprise an RBM of MMTV env, thereby conferring upon a protein an affinity for a TfR. In another embodiment, any protein can be engineered to comprise an RBM. The RBM is, in one embodiment, be inserted using any of the subcloning techniques described herein. Each technique represents a separate embodiment of the present invention. The present invention demonstrated that the RBM of MMTV env binds to TfR1 by showing that mutating the RBM abrogated binding of MMTV env to TfR1 (Example 10).

In one embodiment, the TfR is a mouse TfR. In another embodiment, the TfR is a human TfR. In another embodiment, the TfR is a TfR from another species. In another embodiment, the RBM of MMTV env is modified so that it can interact with a TfR of a species other than mouse. In one embodiment, the modification confers upon a virus comprising the protein of interest the ability to infect, enter, or bind to human cells. In another embodiment, the modification confers upon MMTV env protein an ability to bind to, neutralize, or detect a human TfR or a TfR of a species other than mouse. Many applications exist for the detection, binding to, or neutralization of TfR. In one embodiment, detection of TfR is used to diagnose a disease that affects expression level or abundance of TfR. The disease is an anemia, malarial infection, or any other disease known in the art to affect expression level or abundance of TfR. In another embodiment, detection of TfR (either cell surface-bound TfR or soluble TfR) is used to detect a pathogen that expresses TfR or a homologue thereof. The pathogen is Trypanosoma, or any other pathogen known in the art to expresses TfR or a homologue thereof. Each application represents a separate embodiment of the present invention.

In another embodiment, neutralizing TfR is used to treat a disease or disorder, caused by excess iron loading. In another embodiment, excess iron loading contributes to the progress of the disease or disorder. In another embodiment, excess iron loading contributes to the maintenance of the disease state or disorder. TfR mediates the uptake of iron into cells of the body, known as “iron loading”; thus, in one embodiment, its neutralization reduces iron loading. In another embodiment, the disease or disorder is a cancer, a neoplasia, atherosclerosis, arrhythmia/cardiomyopathy, arthropathy, cirrhosis, rheumatoid arthritis, diabetes, pancreas necrosis, osteoporosis, Parkinson's disease, or any disease or disorder known in the art in which excess iron loading is involved. Each disease or disorder represents a separate embodiment of the present invention.

In another embodiment, neutralizing TfR is used to treat an infection by a pathogen that requires excess iron loading. In another embodiment, neutralizing TfR is used to treat an infection sensitive to a decrease in intracellular iron concentrations. In one embodiment, the pathogen is Listeria Monocytogenes, Salmonella Typhimurium, viral hepatitis, leprosy, or any other pathogen known in the art to require intracellular iron.

In another embodiment, neutralizing TfR is used to treat an infection by a pathogen that expresses TfR or a homologue thereof. Neutralizing the pathogen's TfR decreases, in one embodiment, the viability of the pathogen. In another embodiment, neutralization decreases or abrogates pathogenicity of the pathogen. In one embodiment, the pathogen is Trypanosoma or any other pathogen known in the art to express TfR or a homologue thereof.

In another embodiment, the present invention provides a method of conferring upon a viral particle an increased ability to infect a cell expressing a TfR, comprising pseudotyping the viral particle with a protein comprising an RBM of MMTV env, thereby conferring upon a viral particle an increased ability to infect a cell expressing a TfR. The cell expressing TfR is, in one embodiment, a cancer cell or neoplastic cell. In one embodiment, the cancer cell is a neuroblastoma cell. Many types of cancer cells and neoplastic cells are known to express TfR.

In another embodiment, the present invention provides a method for enhancing an ability of a recombinant retroviral or lentiviral particle to infect a target cell, comprising contacting the target cell with an inhibitor of a lysosomal protease, whereby the inhibitor of a vacuolar enzyme prevents or impedes intracellular degradation of the recombinant retroviral or lentiviral particle, thereby enhancing delivery of a recombinant retroviral or lentiviral particle to a target cell.

The present invention demonstrates that inhibiting cathepsins increases the ability of MoMLV-based vectors to infect cells (Example 4).

In another embodiment, the vacuolar enzyme is a lysosomal enzyme. In another embodiment the vacuolar enzyme is a protease. In another embodiment, the vacuolar enzyme is a cathepsin. In another embodiment, the vacuolar enzyme is any enzyme that resides in a vacuolar compartment or acidic compartment of a cell. Each enzyme represents a separate embodiment of the present invention.

Cathepsins and strategies to inhibit cathepsins are well known in the art, and are reviewed, for example, in Bromme D et al, Curr Pharm Des. 8:1639-58 (2002); Baldwin E T et al, Proc. Natl. Acad. Sci, USA 90:6796-6800 (1993); and Mixuochi T et al Immunol. Lett, 43:189-193 (1994). Each strategy) represents a separate embodiment of the present invention.

In another embodiment, the inhibitor of a vacuolar enzyme is cathepsin inhibitor I Z-Phe-Gly-NHO-Bz (CATI-1) (Demuth et al. (1996) Biochim. Biophys. Acta., 1295:179-186). CATI-1 can be purchased from Calbioclhem, La Jolla, Calif. In another embodiment, the inhibitor is (quinoline-2-carboxylic acid {(S)-3-methyl-1-[(2,2,4-trideuterio)-3-oxo-1-(1-oxy-pyridine-2-sulfonyl)-azepan-4-ylcarbamoyl]-butyl}amide); quinoline-2-carboxylic acid {(S)-3-methyl-1-[3-oxo-1-(1-oxy-pyridine-2-sulfony)-azepan-4-ylcarbamoyl]-butyl}-amide; or N-(1-naphthalenesulfonyl)-L-isoleucyl-L-tryptophanal. In another embodiment, the inhibitor is any cathepsin inhibitor, such as, for example, those disclosed in U.S. Pat. Nos. 6,605,589, 6,597,615, 6,534,498, 6,458,760, 5,955,491, 5,716,980, 5,698,519, 5,639,781, 5,550,138, 5,498,728, and 4,760,130, and US Patent Application 20010056180. Each cathepsin inhibitor represents a separate embodiment of the present invention.

In another embodiment of the present invention, the retroviral or lentiviral particle comprises an env protein that is sensitive to lysosomal degradation. In another embodiment, the env protein is MoMLV env protein (Example 4). In another embodiment, the env protein is a MLV env protein. In another embodiment, the en, protein is a gamma retrovirus env protein. In another embodiment, the env protein is from any virus known in the art. Each env protein represents a separate embodiment of the present invention.

In various embodiments, the inhibitor of a vacuolar enzyme is hirulog; an apis mellifera chymotrypsin/cathpsin G inhibitor; Z-Phe-Gly-NHO-Bz-p-Me; cystatin B; calpain inhibitor II; calpeptin; 3,4 dichloroisocoumarin; NapSul-Ile-Trp-CHO; leupeptin; pepstatin A; Z-F-FMK, or any other inhibitor known in the art. The use of inhibitors of vacuolar enzymes is well known in the art, and is described, for example, in Friedrich B et al, Eur. J. Cancer, 35: 138-144 (1999) Each inhibitor represents a separate embodiment of the present invention.

In another embodiment, the present invention provides a method for enhancing delivery of a nucleic acid sequence in a recombinant retroviral or antiviral particle to a target cell, comprising contacting the target cell with a. the recombinant retroviral or lentiviral particle; and b. an inhibitor of intra-vacuolar acidification, whereby the inhibitor of intra-vacuolar acidification prevents or impedes intracellular degradation of the recombinant retroviral or lentiviral particle, thereby enhancing delivery of a recombinant retroviral or lentiviral particle to a target cell.

In one embodiment, inhibiting intra-vacuolar acidification enhances delivery of a nucleic acid sequence by reducing or eliminating activity of a protease. In one embodiment, the protease is a cathepsin. Caspases, which are dependent on acidic pH for maximal activity, were shown by the present invention to prevent infection of cells by viral particles by degrading the viral particles (Example 4). Caspases are known to require acidic pH for optimum function.

In one embodiment, the inhibitor of intra-vacuolar acidification is bafilomycin. In another embodiment, the inhibitor of intra-vacuolar acidification is concanamycin, ouabain, monensin, nigericin, concanomycin A, or any other inhibitor of intra-vacuolar acidification known in the art. The use of inhibitors of intra-vacuolar acidification is well known, in the art, and is described, for example, in Drose S et al, Journal of Experimental Biology, 200:1-8 (1997). Each inhibitor represents a separate embodiment of the present invention.

In another embodiment, the present invention provides an isolated nucleic acid encoding for a heparin-binding motif of an MMTV env protein, the isolated nucleic acid having a nucleotide sequence selected from the following sequences:

(SEQ ID No 27)

ATAAAGAAGAAGTTGCCCCCCAAATAT;

(SEQ ID No 28)

CCTATAAAGAAGAAGTTGCCCCCCAAATATCCT;

(SEQ ID No 29)

GTCCCTATAAAGAAGAAGTTGCCCCCCAAATATCCTCAC;

(SEQ ID No 30)

CTGGTCCCTATAAAGAAGAAGTTGCCCCCCAAATATCCTCACTGC;

(SEQ ID No 31)

AAACTGGTCCCTATAAAGAAGAAGTTGCCCCCCAAATATCCTCACTGC;

(SEQ ID No 32)

CCTGGGGGAAAGGGTGATAAAAGGCGTATGTGGGAACTCTGGTTGACTAC
TT;

(SEQ ID No 56)

AAGAAGAAGTTGCCCCCCAAA;

(SEQ ID No 57)

ACAAAACTGGTCCCTATAAAGAAGAAGTTGCCCCCCAAATATCCT;

(SEQ ID No 58)

CCTGGGGGAAAGGGTGATAAAAGGCGTATGTGGGAACTCTGGTTGACTAC
TTTGGGGAACTCAGGGGCCAATACAAAACTGGTCCCTATAAAGAAGAAGT
TGCCCCCCAAATATCCT;

(SEQ ID No 59)

AAGGGTGATAAAAGGCGTATGTGGGAACTCTGGTTGACTACTTTGGGGAA
CTCAGGGGCCAATACAAAACTGGTCCCTATAAAGAAGAAGTTGCCCCCCA
AA;

(SEQ ID No 60)

CCTGGGGGAAAGGGTGATAAAAGGCGTATGTGGGAACTCTGGTTGACTAC
TTTGGGGAACTCAGGG;

(SEQ ID No 61)

AAACCTGGGGGAAAGGGTGATAAAAGGCGTATGTGGGAACTCTGGTTGAC
TACTTTGGGGAACTCAGGGGCCAATACAAAACTGGTCCCTATAAAGAAGA
AGTTGCCCCCCAAATATCCTCACTGCCAGATCGCCTTTAAGAAGGACGCC
TTCTGGGAGGGAGACGAGTCTGCTCCTCCACGGTGGTTGCCT;
and

(SEQ ID No 82)

AAGGGTGATAAAAGGCGT

In another embodiment, the sequence of the isolated nucleic acid is homologous to one of the above nucleotide sequences.

In another embodiment, the present invention provides recombinant nucleic acid molecule comprising a heterologous nucleotide, the heterologous nucleotide corresponding to an isolated nucleic acid of the present invention that encodes for a heparin-binding motif.

An HBM is, in one embodiment, a region of an env protein that mediates interaction with a heparin molecule. In another embodiment, binding to heparin contributes to infection. In another embodiment, binding to heparin attaches a viral particle to a cell without contributing to infection. In another embodiment, an HBM binds to a heparin molecule that is not associated wait a target cell.

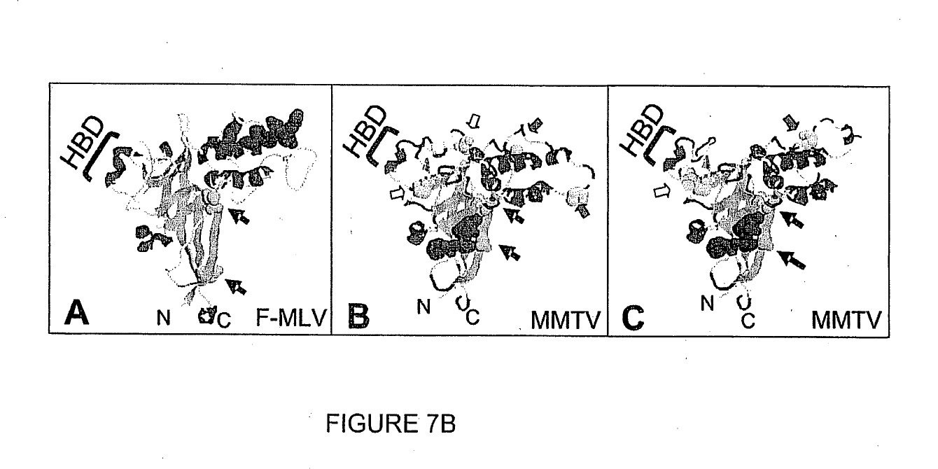

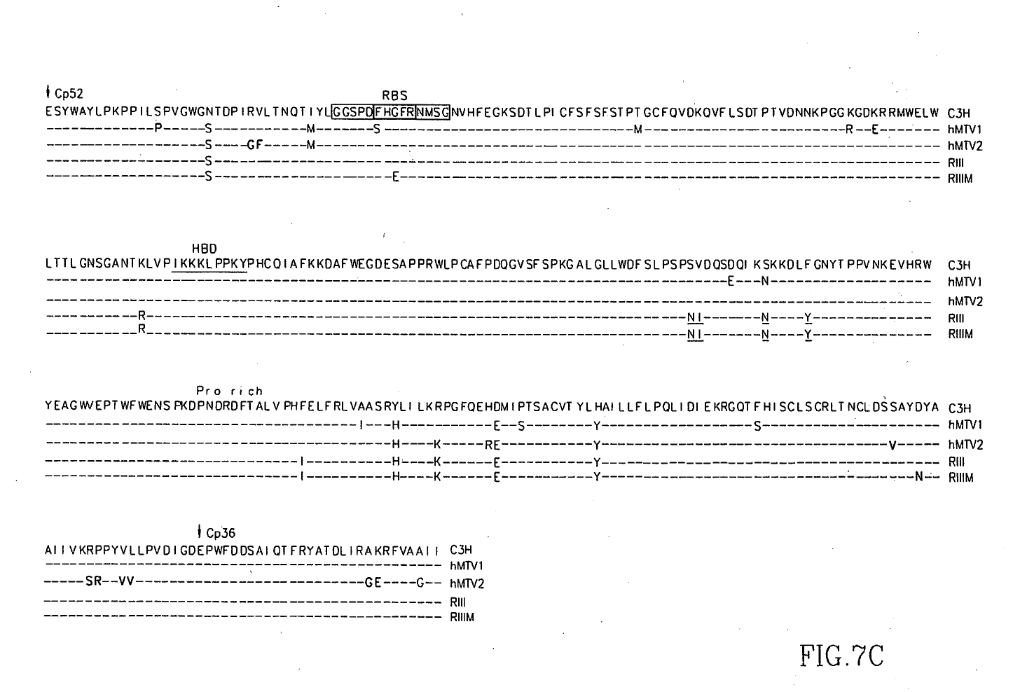

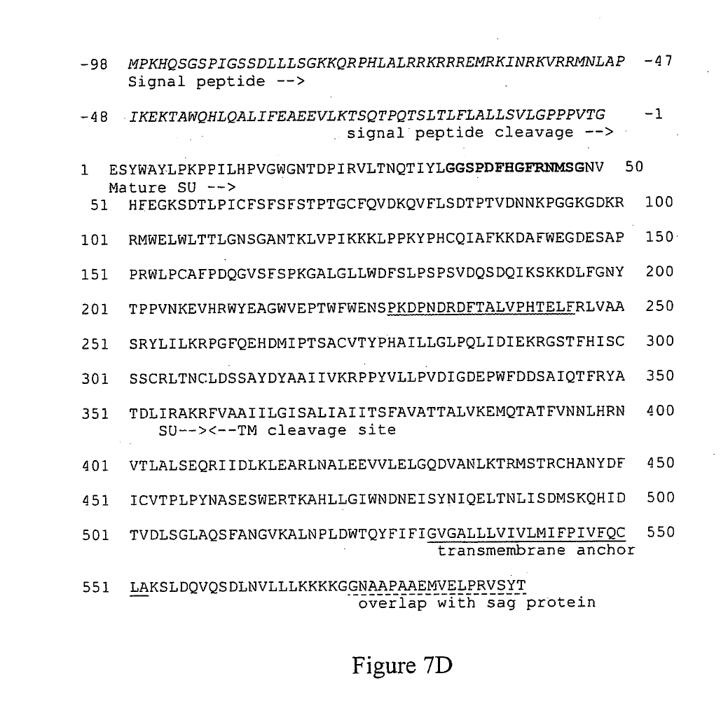

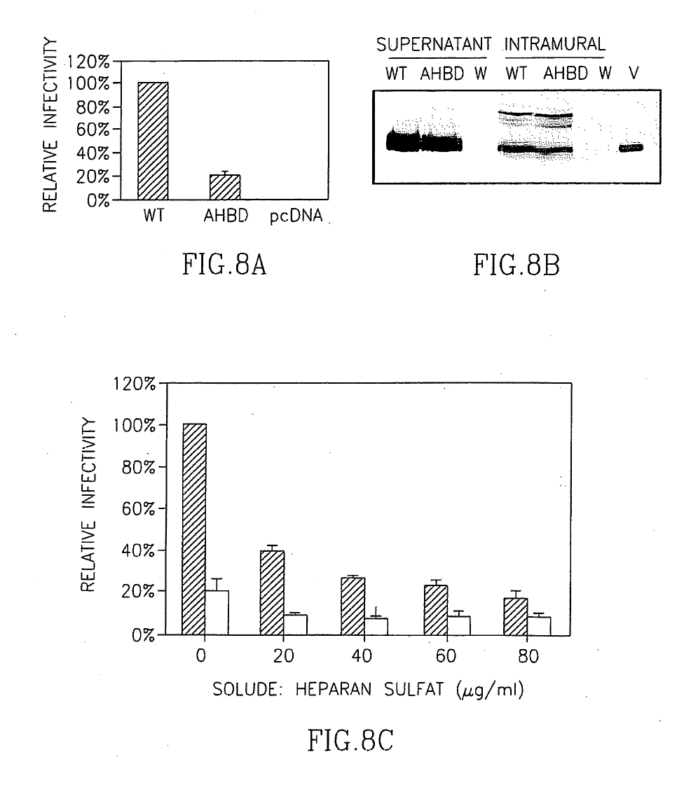

The present invention identified an HBM in MMTV env protein by sequence and structural alignment with Friend ecotropic MLV (F-MLV) env protein, and confirmed the identification by an alignment of the predicted structures of MMTV env protein and F-MLV env protein (FIG. 7). The HBM was shown to be necessary for infection by the finding, of the present invention that deletion of the HBM sharply reduced the infectivity of MMTV (FIG. 8).

In one embodiment, the heparin molecule is on the surface of the target cell. In another embodiment, the heparin molecule resides in an internal membrane of the target cell.

In another embodiment, the present invention provides an isolated polypeptide comprising a heparin-binding motif (HBM) of an MMTV env protein.

In another embodiment, the present invention provides a method of decreasing or abrogating binding of a viral particle to a cell with heparan sulfate proteoglycan molecules on its surface, comprising contacting the cell or viral particle with an agent that blocks, binds to, or interacts with an HBM of an MMTV env protein, the HBM having a sequence selected from the sequences set forth in SEQ ID No 33-8 and 62-69, thereby decreasing or abrogating binding of a viral particle to a cell with heparan sulfate proteoglycan molecules on its surface.

In one embodiment, the HBM of the env protein corresponds to the following residues of MMTV env protein: IKKKLPPKY (SEQ ID No 33); or, in another embodiment, the residues: PIKKKLPPKYP (SEQ ID No 34); or, in another embodiment, the residues: Val Pro Ile Lys Lys Lys Leu Pro Pro Lys Tyr Pro His (SEQ ID No 35); or, in another embodiment, the residues: Leu Val Pro Ile Lys Lys Lys Leu Pro Pro Lys Tyr Pro His Cys (SEQ ID No 36); or, in another embodiment, the residues: Lys Leu Val Pro Ile Lys Lys Lys Leu Pro Pro Lys Tyr Pro His Cys Gin (SEQ ID No 37); or, in another embodiment, the residues: Thr Lys Leu Val Pro Ile Lys Lys Lys Leu Pro Pro Lys Tyr Pro His Cys Gln Ile (SEQ ID No 38); or, in another embodiment, the residues: KKKLPPK (SEQ ID No 62); or, in another embodiment, the residues: TKLVPIKKKLPPKYP (SEQ ID No 63); or, in another embodiment, the residues: PGGKGDKRRMWELWLTTLGNSGANTKLVPIKKKLPPKYP (SEQ ID No 64); or, in another embodiment, the residues: KGDKRRMWELIAILTTLGNSGANTKLVPIKKKLPPK (SEQ ID No 65); or, in another embodiment, the residues: KGDKRR (SEQ ID No 66); or, in another embodiment, the residues: PGGKGDKRRMWELWLTTLG (SEQ ID No 67); or, in another embodiment, the residues: PGGKGDKRRMWELWLTTLGNSG (SEQ ID No 68); or, in another embodiment, the residues: KPGGKGDKRRMWELWLTTLGNSGANTKLVPIKKKLPPKYPHCQIAFKKDAFWEGDESAPPRWLP (SEQ ID No 69) In another embodiment, the HBM of the protein corresponds to a group of residues of MMTV env protein approximately centered around residues 122-133 of SEQ ID No 10. In another embodiment, the HBM of the protein is homologous to an HBM sequence of the present invention.

In another embodiment, the present invention provides an isolated nucleic acid, comprising a nucleic acid sequence encoding for a mutated HBM of an MMTV or MoMLV env protein. Each type of mutation described herein for the RBM is performed on the HBM, and each represents a separate embodiment of the present invention.

In another embodiment, the present invention provides a method of decreasing or abrogating binding of a viral particle to a cell, comprising contacting the cell or viral particle with an agent that blocks, binds to, or interacts with an RBM of an MMTV env protein. In another embodiment, the present invention provides a method of decreasing or abrogating binding of a viral particle to a cell, comprising contacting the cell or viral particle with an agent that blocks, binds to, or interacts with an HBM of an MMTV env protein. Blocking, binding to, or interacting with the RBM or HBM decreases or abrogates, in these embodiments, interaction between the RBM or HBM and a cellular molecule, thereby decreasing or abrogating binding of a viral particle to a cell. In one embodiment, the cellular molecule is TfR, heparin, or any other molecule known in the art that interacts with the RBM or HBM. In another embodiment, this method is used to decrease or abrogate entry of a target cell by a viral particle. The agent is an antibody or other any other type of compound or composition. Each type of agent represents a separate embodiment of the present invention.

In another embodiment, the present invention provides a method of conferring upon a viral particle an increased ability to infect a cell comprising heparin, comprising pseudotyping the viral particle with a protein comprising an HBM of MMTV env, thereby conferring upon a viral particle an increased ability to infect a cell comprising heparin.

Findings of the present invention also show that recombinant viral env proteins of the present invention can utilize cellular targets that are internalized via an endocytic uptake pathway different from the pathway utilized by the wild-type env protein from which the recombinant env protein was derived (Example 16). Wild-type ecotropic MoMLV enters cells via a non-clathrin-mediated endocytic pathway using the natural ecotropic MLV receptor, cationic amino acid transporter. Wild-type MMTV enters cells via clathrin coated pits (clathrin mediated endocytosis), using its natural receptor, mouse Transferrin Receptor-1. Prior to the findings of the present invention, it was believed that viral env proteins that ordinarily utilize one endocytic pathway cannot be engineered to enter cells via a different endocytic pathway.

The possibility of doing so was shown in the present invention using Somatostatin (Sst-14). Sst-14 utilizes a family of five different receptors; which all are G protein-coupled receptors (GPCRs) but differ in their endocytosis, desensitization and recycling following Sst-14 binding. GPCR are 7-transmembrane proteins whose signaling can be down-regulated. The GPCRs are re-sensitized by uncoupling their association with bound ligand in the following process: Phosphorylation of their intracellular domains during activation recruits cytoplasmic proteins called arresting. Arrestin binding cross-links the uncoupled GPCR to components of the endocytic machinery such as AP-2 and clathrin. The receptor is endocytosed, stripped of ligand, dephosphorylated and recycled to the plasma membrane.

GPCRs have been categorized into two classes based on the isoforms of arrestins that they bind:

Class A: do not bind visual arrestins; bind nonvisual arrestins with greater affinity for β-arrestin-2 than for β-arrestin-1; and are directed to clathrin-coated pits by β-arrestins but the arrestins do not internalize with the receptors.

Class B: bind visual arrestin: bind β-arrestin-1 and β-arrestin-2 with similar affinities; and β-arrestins internalize into early endosomes in complex with GPCRs but not clathrin. Somatostatin receptors SSTR2A and SSTR3 are class B receptors and SSTR5 is a class A receptor

In another embodiment, the present invention provides a method for delivering a nucleic acid of interest or compound of interest to a target cell via a clathrin-independent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-dependent endocytosis, and wherein the mutated version of a wild-type env protein comprises an insertion of a heterologous peptide that binds a cellular surface protein that capable of being internalized via a clathrin-independent endocytosis, thereby delivering a nucleic acid of interest or compound of interest to a target cell via a clathrin-independent endocytosis.

In another embodiment, the present invention provides a method for delivering a nucleic acid of interest or compound of interest to a target cell via an endocytosis pathway that leads to caveosomes, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-dependent endocytosis, and wherein the mutated version of a wild-type env protein comprises an insertion of a heterologous peptide that binds a cellular surface protein that capable of being internalized via a clathrin-independent endocytosis, thereby delivering a nucleic acid of interest or compound of interest to a target cell via an endocytosis pathway that leads to caveosomes.

In another embodiment, the present invention provides a method for delivering a nucleic acid of interest or compound of interest to a target cell via a clathrin-independent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via an endocytosis pathway that leads to endosomes or an acidic compartment, and wherein the mutated version of a wild-type env protein comprises an insertion of a heterologous peptide that binds a cellular surface protein that capable of being internalized via a clathrin-independent endocytosis, thereby delivering a nucleic acid of interest or compound of interest to a target cell via a clathrin-independent endocytosis.

In another embodiment, the present invention provides a method for delivering a nucleic acid of interest or compound of interest to a target cell via a clathrin-dependent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-independent endocytosis, and wherein the mutated version of a wild-type env protein comprises an insertion of a heterologous peptide that binds a cellular surface protein that capable of being internalized via a clathrin-dependent endocytosis, thereby delivering a nucleic acid of interest or compound of interest to a target cell via a clathrin-dependent endocytosis.

In another embodiment, the present invention provides a method for delivering a nucleic acid of interest or compound of interest to a target cell via a clathrin-dependent endocytosis, comprising contacting the target cell with a recombinant viral particle comprising (a) a nucleic acid of interest or compound of interest; and (b) a mutated version of a wild-type env protein, wherein viruses containing the wild-type env protein are internalized via an endocytosis pathway that leads to caveosomes, and wherein the mutated version of a wild-type env protein comprises an insertion of a heterologous peptide that binds a cellular surface protein that capable of being internalized via a clathrin-dependent endocytosis, thereby delivering a nucleic acid of interest or compound of interest to a target cell via a clathrin-dependent endocytosis.

In another embodiment, the present invention provides a method for delivering a nucleic acid of interest or compound of interest to a target cell via an endocytosis pathway that leads to endosomes or an acidic compartment, comprising contacting the target cell with a recombinant viral particle comprising (a) a nucleic acid of interest or compound of interest; and (b) a mutated version of a wild-type env protein, wherein viruses containing the wild-type env protein are internalized via a clathrin-independent endocytosis, and wherein the mutated version of a wild-type env protein comprises an insertion of a heterologous peptide that binds a cellular surface protein that capable of being internalized via a clathrin-dependent endocytosis, thereby delivering a nucleic acid of interest or compound of interest to a target cell via an endocytosis pathway that leads to endosomes or an acidic compartment.

In another embodiment, the present invention provides a method for enhancing an ability of a recombinant viral env protein to mediate infection of a target cell, wherein said recombinant viral env protein is derived from a wild-type viral env protein that is capable of mediating internalization via a clathrin-independent endocytosis, comprising engineering said recombinant viral env protein to comprise a heterologous peptide, whereby said heterologous peptide binds a cellular surface protein that capable of being internalized via a clathrin-dependent endocytosis, thereby enhancing an ability of a recombinant viral env protein to mediate infection of a target cell.

In another embodiment, the present invention provides a method for enhancing an ability of a recombinant viral env protein to mediate infection of a target cell, wherein said recombinant viral env protein is derived from a wild-type viral env protein that is capable of mediating internalization via a clathrin-dependent endocytosis, comprising engineering said recombinant viral env protein to comprise a heterologous peptide, whereby said heterologous peptide binds a cellular surface protein that capable of being internalized via a clathrin-independent endocytosis, thereby enhancing an ability of a recombinant viral env protein to mediate infection of a target cell.

In another embodiment, the present invention provides a method for enhancing an ability of a wild-type viral env protein to mediate infection of a target cell, wherein said wild-type viral env protein is capable of mediating internalization via a clathrin-independent endocytosis, comprising engineering said wild-type viral env protein to comprise a heterologous peptide, whereby said heterologous peptide binds a cellular surface protein that capable of being internalized via a clathrin-dependent endocytosis, thereby enhancing an ability of a wild-type viral env protein to mediate infection of a target cell.

In another embodiment, the present invention provides a method for enhancing an ability of a wild-type viral env protein to mediate infection of a target cell, wherein said wild-type viral env protein is capable of mediating internalization via a clathrin-dependent endocytosis, comprising engineering said wild-type viral env protein to comprise a heterologous peptide, whereby said heterologous peptide binds a cellular surface protein that capable of being internalized via a clathrin-independent endocytosis, thereby enhancing an ability of a wild-type viral env protein to mediate infection of a target cell.

In another embodiment, the clathrin-dependent endocytosis of the above methods is an endocytosis via a clathrin-coated pit. In another embodiment the clathrin-dependent endocytosis is any other type of clathrin-dependent endocytosis known in the art. In another embodiment, the clathrin-independent endocytosis of the above methods is an endocytosis via a caveolae or a caveolin-coated pit. In another embodiment, the clathrin-independent endocytosis is any other type of clathrin-independent endocytosis known in the art. Each possibility represents a separate embodiment of the present invention.

In another embodiment, the recombinant viral env protein or wild-type viral env protein of one of the above methods is sensitive to degradation by acidic pH in endosomes or lysosomes. In another embodiment, the recombinant viral env protein or wild-type viral env protein is not sensitive to degradation by acidic pH in endosomes or lysosomes. In another embodiment, the recombinant viral env protein or wild-type viral env protein of one of the above methods fuses with cellular membranes in a pH-dependent fashions. In another embodiment, the recombinant viral env protein or wild-type viral env protein of one of the above methods fuses with cellular membranes in a pH-independent fashion. Each possibility represents a separate embodiment of the present invention.

In another embodiment, the present invention provides a method of conferring upon a protein an affinity for heparin, comprising engineering the protein of interest to comprise an HBM of MMTV env, as set forth in SEQ ID No 33-38 and 62-69, thereby conferring upon a protein an affinity for heparin. In principle, any protein can be engineered to comprise an HBM. In one embodiment, the HBM may be inserted using any of the techniques described above for subcloning. Each technique represents a separate embodiment of the present invention.

In another embodiment, the present invention provides a composition comprising an isolated nucleic acid, polypeptide, vector, cell, or packaging cell line of the present invention. In one embodiment, the composition comprises a liposome or other vehicle for introducing the isolated nucleic acid into a cell or for introducing the nucleic acid into a patient.

In another embodiment, the present invention provides a method for delivering a nucleic acid of interest to a target cell, comprising contacting the target cell with a recombinant viral particle or liposome comprising: (a) a nucleic acid of interest; and (b) a mutant or variant MMTV env protein, comprising a mutation in a nucleic acid sequence encoding for the MMTV env protein, whereby the mutation in the MMTV env protein mediates uptake of the recombinant viral particle or liposome via a cellular molecule and via fusion of a membrane of the target cell with a membrane of the recombinant viral particle, thereby delivering a nucleic acid of interest to a target cell. In the case of MMTV env protein, the mutation need not be in the RBM, RBD, or HBD to change the binding specificity of the virus. Any type of mutation of the present invention may be utilized in mutating MMTV env protein, and each type represents an embodiment of the present invention.

In another embodiment, the present invention provides a method of delivering a compound of interest to an acidified compartment of a target cell, comprising a. chemically attaching the compound of interest to a mutated MMTV env protein that is directed to the acidified compartment to form a mutated MMTV env protein-compound complex; and b. contacting the target cell with the mutated MMTV env protein-compound complex. In this method, the mutated MMTV env protein is directed to the acidified compartment via interaction with a surface molecule of the target cell that is itself routed to the acidified compartment, thereby delivering a compound of interest to an acidified compartment of a target cell. In the case of MMTV env protein, the mutation need not be in the RBM, RBD, or HBD to direct the protein to an acidified compartment of a target cell.

In another embodiment, the present invention provides a method for delivering a nucleic acid of interest to a mouse target cell, comprising contacting the target cell with a recombinant viral particle or liposome comprising (a) an MMTV env protein, comprising a receptor-binding motif as set forth in SEQ ID No 11-16 and 51-55; (b) a genome of a virus other than 4MTV; and (c) the nucleic acid of interest, whereby an interaction between the receptor-binding motif and the target cell mediates intracellular uptake of the recombinant viral particle or liposome, thereby delivering a nucleic acid of interest to a target cell.

In another embodiment, the present invention provides a method for delivering a nucleic acid of interest to a target cell, comprising contacting the target cell with a recombinant viral particle or liposome comprising (a) a nucleic acid of interest; and (b) a mutant or variant retroviral or lentiviral env protein, comprising a mutation in a nucleic acid sequence encoding for a receptor-binding motif of the mutant or variant retroviral or lentiviral env protein, whereby the mutation in the nucleic acid sequence mediates uptake of the recombinant viral particle or liposome via a cellular molecule and via fusion of a membrane of the target cell with a membrane of the recombinant viral particle or liposome, thereby delivering a nucleic acid of interest to a tar-et cell.

In another embodiment, methods of the present invention further comprise pseudotyping an env protein of the present invention with a cytoplasmic tail of a different env protein. In another embodiment, sequence from the cytoplasmic tail of the different env protein replaces all or part of the sequence encoding the cytoplasmic tail of the env protein of the present invention. In another embodiment, the sequence is inserted into the gene encoding the env protein of the present invention. In one embodiment, such pseudotyping increases infectivity of a virus comprising the mutant protein. In another embodiment, a cytoplasmic tail from a protein other than MoMLV env protein increases production of a virus comprising the mutant protein. In another embodiment, the cytoplasmic tail increases incorporation of the mutant protein into a virus. In another embodiment, the cytoplasmic tail increases incorporation of a nucleic acid into a virus. In another embodiment, the cytoplasmic tail alters specificity of incorporation of nucleic acid into a virus. In another embodiment, the cytoplasmic tail alters any other desired characteristic of the mutant protein or a recombinant virus comprising same. Each possibility represents a separate embodiment of the present invention.

### EXPERIMENTAL DETAILS SECTION

### Example 1

### Replacement of the RBM of MoMLV env Protein with an Sst Peptide Confers Upon Pseudotyped Virus Ability to Infect Cells Expressing SstR

### Materials and Experimental Methods

Construction of Mutant MoMLV Env Sequences

Recombinant MoMLV Env sequences were produced using the QuikChange site site-directed mutagenesis kit (Strategene, Inc.) MoMLV-Sst-RBM1 was produced by replacing nucleotide bases 412-454 with a nucleotide encoding a somatostatin sequence, TACGCGTCGGCTGGCTGCAAGAATTTCTTCTGGAAGACTTTCACTAGTTGCGCGTATACCGCGTCC (SEQ ID No 1) into a MoMLV env gene (SEQ ID No 39, as delineated hereinabove), yielding the sequence:

ATGGCGCGTTCAACGCTCTCAAAACCCCTTAAAAATAAGGTTAACCCGCG
AGGCCCCCTAATCCCCTTAATTCTTCTGATGCTCAGAGGGGTCAGTACTG
CTTCGCCCGGCTCCAGTCCTCATCAAGTCTATAATATCACCTGGGAGGTA
ACCAATGGAGATCGGGAGACGGTATGGGCAACTTCTGGCAACCACCCTCT
GTGGACCTGGTGGCCTGACCTTACCCCAGATTTATGTATGTTAGCCCACC
ATGGACCATCTTATTGGGGGCTAGAATATCAATCCCCTTTTTCTTCTCCC
CCGGGGCCCCCTTACGCGTCGGCTGGCTGCAAGAATTTCTTCTGGAAGAC
TTTCACTAGTTGCGCGTATACCGCGTCCGAAGAACCTTTAACCTCCCTCA
CCCCTCGGTGCAACACTGCCTGGAACAGACTCAAGCTAGACCAGACAACT
CATAAATCAAATGAGGGATTTTATGTTTGCCCCGGGCCCCACCGCCCCCG
AGAATCCAAGTCATGTGGGGGTCCAGACTCCTTCTACTGTGCCTATTGGG
GCTGTGAGACAACCGGTAGAGCTTACTGGAAGCCCTCCTCATCATGGGAT
TTCATCACAGTAAACAACAATCTCACCTCTGACCAGGCTGTCCAGGTATG
CAAAGATAATAAGTGGTGCAACCCCTTAGTTATTCGGTTTACAGACGCCG
GGAGACGGGTTACTTCCTGGACCACAGGACATTACTGGGGCTTACGTTTG
TATGTCTCCGGACAAGATCCAGGGCTTACATTTGGGATCCGACTCAGATA
CCAAAATCTAGGACCCCGCGTCCCAATAGGGCCAAACCCCGTTCTGGCAG
ACCAACAGCCACTCTCCAAGCCCAAACCTGTTAAGTCGCCTTCAGTCACC
AAACCACCCAGTGGGACTCCTCTCTCCCCTACCCAACTTCCACCGGCGGG
AACGGAAAATAGGCTGCTAAACTTAGTAGACGGAGCCTACCAAGCCCTCA
ACCTCACCAGTCCTGACAAAACCCAAGAGTGCTGGTTGTGTCTAGTAGCG
GGACCCCCCTACTACGAAGGGGTTGCCGTCCTGGGTACCTACTCCAACCA
TACCTCTGCTCCAGCCAACTGCTCCGTGGCCTCCCAACACAAGTTGACCC
TGTCCGAAGTGACCGGACAGGGACTCTGCATAGGAGCAGTTCCCAAAACA
CATCAGGCCCTATGTAATACCACCCAGACAAGCAGTCGAGGGTCCTATTA
TCTAGTTGCCCCTACAGGTACCATGTGGGCTTGTAGTACCGGGCTTACTC
CATGCATCTCCACCACCATACTGAACCTTACCACTGATTATTGTGTTCTT
GTCGAACTCTGGCCAAGAGTCACCTATCATTCCCCCAGCTATGTTTACGG
CCTGTTTGAGAGATCCAACCGACACAAAAGAGAACCGGTGTCGTTAACCC
TGGCCCTATTATTGGGTGGACTAACCATGGGGGGAATTGCCGCTGGAATA
GGAACAGGGACTACTGCTCTAATGGCCACTCAGCAATTCCAGCAGCTCCA
AGCCGCAGTACAGGATGATCTCAGGGAGGTTGAAAAATCAATCTCTAACC
TAGAAAAGTCTCTCACTTCCCTGTCTGAAGTTGTCCTACAGAATCGAAGG
GGCCTAGACTTGTTATTTCTAAAAGAAGGAGGGCTGTGTGCTGCTCTAAA
AGAAGAATGTTGCTTCTATGCGGACCACACAGGACTAGTGAGAGACAGCA
TGGCCAAATTGAGAGAGAGQCTTAATCAGAGACAGAAACTGTTTGAGTCA
ACTCAAGGATGGTTTGAGGGACTGTTTAACAGATCCCCTTGGTTTACCAC
CTTGATATCTACCATTATGGGACCCCTCATTGTACTCCTAATGATTTTGC
TCTTCGGACCCTGCATTCTTAATCGATTAGTCCAATTTGTTAAAGACAGG
ATATCAGTGGTCCAGGCTCTAGTTTTGACTCAACAATATCACCAGCTGAA
GCCTATAGAGTACGAGCCA (SEQ ID No 41; inserted se-
quence underlined).

The above nucleotide sequence encodes the amino acid sequence:

MARSTLSKPLKLNKVNPRGPLIPLILLMLRGVSTASPGSSPHQVYNVFWE
VTNGDRFETVWATSGNHPLWTWWPDLTPDLCMLAHHGPSYWGLEYQSPFS
SPPGPPYASAGCKNFFWKTFTSCYTASEEPLTSLTPRCNTAWNRLKLDQT
THKSNEGFYVCPGPHRPRESKSCGGPDSFYCAYWGCETTGRAYWKPSSSW
DFITVNNNLTSDQAVQVCKDNKWCNPLVIRFTDAGRRVTSWTTGHYWGLR
LYVSGQDPGLTFGIRLRYQNLGPRVPIGPNPVLADQQPLSKPKPVKSPSV
TKPPSGTPLSPTQLPPAGTENRLLNLVDGAYQALNLTSPDKTQECWLCLV
AGPPYYEGVAVLGTYSNHTSAPANCSVASQHKLTLSEVTGQGLCIGAVPK
THQALCNTTQTSSRGSYYLVAPTGTMWACSTGLTPCISTTILNLTTDYCV
LVELWPRVTYHSPSYVYGLFFRSNRHKREPVSLTLALLLGGLTMGGIAAG
IGTGTTALMATQQFQQLQAAVQDDLREVEKSISNLEKSLTSLSEVVLQNR
RGLDLLFLKEGGLCAALKEECCFYADHTGLVRDSMAKLRERLNQRQKLFE
STQGWFEGLFNRSPWFTTLISTIMGPLIVLLMILLFGPCILNRLVQFVKD
RISVVQALVLTQQYHQLKPIEYEP (SEQ ID No 42; inserted
sequence underlined).

Sst-PRR (described below) had the sequence:

(SEQ ID No 87)

MARSTLSKPLKNKVNPRGPLIPLILLMLRGVSTASPGSSPHQVYNITWEV
TNGDRETVWATSGNHPLWTWWPDLTPDLCMLAHHGPSYWGLEYQSPFSSP
PGPPCCSGGSSPGCSRDCEEPLTSLTPRCNTAWNRLKLDQTTHKSNEGFY
VCPGPHRPRESKSCGGPDSFYCAYWGCETTGRAYWKPSSSWDFITVNNNL
TSDQAVQVCKDNKWCNPLVIRFTDAGRRVTSWTTGHYWGLRLYVSGQDPG
LTFGTRLRYQNLGPRVPIGPNPVLADQQPLSKPKPVKSPSVTKPPSGGGG
AGCKNFFWKTFTSCSGGGTPLSPTQLPPAGTENRLLNLVDGAYQALNLTS
PDKTQECWLCLVAGPPYYBGVAVLGTYSNHTSAPANCSVASQHKLTLSEV
TGQGLCIGAVPKTHQALCNTTQTSSRGSYYLVAPTGTMWACSTGLTPCLS
TTILNLTTDYCVLVELWPRVTYHSPSYVYGLFERSNRHKREPVSLTLALL
LGGLTMGGIAAGIGTGTTALMATQQFQQLQAAVQDDLRFVEKSISNLEKS
LTSLSEVVLQNRRGLDLLFLKEGGLCAALKEECCFYADHTGLVRDSMAKL
RERLNQRQKLFESTQGWEEGLFNRSPWFTTLISTIMGPLIVLLMILLFGP
CILNRLVQFVDRISVVQALVLTQQYHQLKPIEYEP.

MoMLV Sst-230 env (described below) had the sequence:

(SEQ ID No 88)

MARSTLSKPLKNKVNPRGPLIPLILLMLRGVSTASPGSSPHQVYNITWEV
TNGDRETVWATSGNHPLWTWWPDLTPDLCMLAHHGPSYWGLEYQSPFSSP
PGPPCCSGGSSPGCSRDCEEPLTSLTPRCNTAWNRLKLDQTTHKSNEGFY
VCPGPHRPRESKSCGGPDSFYCAYWGCETTGRAYWKPSSSWDFITVNNNL
TSDQAVQVCKDNKWCNPLVIRFTDAGRRVTSWTTGHYWGLPLYVSGQDPG
LTFGIRLRYQNLGAGCKNFFWKTFTSCPRVPIGPNPVLADQQPLSKPKPV
KSPSVTKPPSGTPLSPTQLPPAGTENRLLNLVDGAYQALNLTSPDKTQEC
WLCLVAGPPYYEGVAVLGTYSNHTSAPANCSVASQHKLTLSEVTGQGLCI
GAVPKTHQALCNTTQTSSRGSYYLVAPTGTMWACSTGLTPCISTTILNLT
TDYCVLVELWPRVTYHSPSYVYGLFFRSNRHKREPVSLTLALLLGGLTMG
GIAAGIGTGTTALMATQQFQQLQAAVQDDLREVEKSISNLEKSLTSLSEV
VLQNRRGLDLLFLKEGGLCAALKEECCFYADHTGLVRDSMAKLRERLNQR
QKLFESTQGWFEGLENRSPWFTTLISTIMGPLIVLLMLLLFGPCILNRLV
QFVKDRISVVQALVLTQQYHQLKPIEYEP.

MoMLV Sst-N env, another construct used in the experiments below, had the sequence:

(SEQ ID No 89)

MARSTLSKPLKNKVNPRGPLIPLILLMLRGVSTSGGGGAGCKNFFWKTFT
SCSGGGASPGSSPHQVYNITWEVTNGDRETVWATSGNHPLWTWWPDLTPD
LCMLAHHGPSYWGLEYQSPFSSPPGPPCCSGGSSPGCSRDCEEPLTSLTP
RCNTAWNRLKLDQTTHKSNEGFYVCPGPHRPRESKSCGGPDSFYCAYWGC
ETTGPAYWKPSSSWDFITVNNNLTSDQAVQVCKDNKWCNPLVIRFTDAGR
RVTSWTTGHYWGLRLYVSGQDPGLTFGIRLRYQNLGPRVPIGPNPVLADQ
QPLSKPKPVKSPSVTKPPSGTPLSPTQLPPAGTENRLLNLVDGAYQALNL
TSPDKTQECWLCLVAGPPYYEGVAVLGTYSNHTSAPANCSVASQHKLTLS
EVTGQGLCIGAVPKTHQALCNTTQTSSRGSYYLVAPTGTMWACSTGLTPC
ISTTILNLTTDYCVLVELWPRVTYHSPSYVYGLFERSNRHKREPVSLTLA
LLLGGLTMGGLAAGIGTGTTALMATQQFQQLQAAVQDDLREVEKSISNLE
KSLTSLSEVVLQNRRGLDLLFLKEGGLCAALKEECCFYADHTGLVRDSMA
KLRERLNQRQKLFESTQGWFEGLFNRSPWFTTLISTLMGPLIVLLMILLF
GPCILNRLVQFVKDRISVVQALVLTQQYHQLKPIEYEP.

The following primers were used to produce MoMLV-Sst-RBM1. First, the nucleotide sequence encoding the RBM (SEQ ID No: 76) of the MoMLV env gene (SEQ ID No 39) was replaced by a short sequence containing a unique Mlul restriction enzyme site using the ExSite mutagenesis kit (Stratagene) with the following primers: TACGCGTCCGAAGAACCTTTAACCTCCCTC (SEQ ID No 83) and AGGGGGCCCCGGGGGAGAAG (SEQ ID No 84), each of which contains an Mlul restriction enzyme site. The resulting mutated nucleic acid was digested with Mlul, then ligated to a Sst peptide-encoding fragment generated by annealing two oligonucleotides:

(SEQ ID No 85)

CGCGTCGGCTGGCTGCAAGAATTTCTTCTGGAAGACTTTCACTAGTTGCG
CGTATAC
and

(SEQ ID No 86)

CGCGGTATACGCGCAACTAGTGAAAGTCTTCCAGAAGAAATTCTTGCAGC
CAGCCGA.

Production of Mutant Viruses

A plasmid encoding wild-type env, gag and pol proteins was constructed by inserting the MoMLV gag pol and env genes into expression vector pcDNA3 (Invitrogen) as described in Zavorotinskaya T. et al (J. Virol. 73:5034-42). Plasmids encoding mutant env proteins and wild type gag and pol proteins were constructed by inserting the NdeI-EcoRI restriction fragment of the mutated MoMLV sequence, containing nucleotide 5403 through the env protein stop codon, in place of the NdeI-EcoRI restriction fragment of the wild-type MoMLV sequence of the gag-pol-env plasmid. Coding sequences on the resulting plasmids were co-expressed with the pBAG plasmid to generate mutant MoMLV viruses expressing an Escherichia coli beta-galactosidase (beta-gal) protein, as described in Zavorotinskaya T et al (J Virol 73: 5034-42). The pBAG plasmid encodes a packageable MoMLV genome lacking gag, pol, or env sequences but comprising a beta-gal gene under the control of the retroviral 5′ long terminal repeat, and comprising the neomycin resistance gene under the simian virus 40 promoter.

Transfection of 293 Cells to Express SstR2

HEK 293 cells (human embryonic kidney 293 cells) were transfected with a vector encoding the human somatostatin receptor type 2 (SstR2) by standard DNA transfection techniques. Transfection was performed by calcium phosphate precipitation as described in Molecular Cloning, (2001), Sambrook and Russell, eds.

Antibodies

Polyclonal goat anti-MMTV or SU antiserum is described in Dzuris, J. L. et al, Virol. 263: 418-426 (1999).

Detection of Infected Cells

For virus infection assays, 2×10⁵ cells were grown on 6-well plates for 1 day and serially diluted pseudovirus supernatants containing polybrene (8 μg/ml) were incubated with cells at 37° C. for two hours. Residual virus was removed by aspiration and replaced with regular growth medium. Forty-eight hours later, the cells were fixed in 0.25% glutaraldehyde in phosphate buffered saline. Beta-gal expression, detected by staining with chromogenic substrate (X-Gal [5-bromo-4-chloro-3-indolyl-D-galactopyranoside]), was used as a marker for cells infected by the recombinant viral particles. Positive cells were identified as blue cells under a light microsope and the virus titer was calculated from the quantification of the end-point dilution. Infectivity data are presented as lacZ-forming units (LFU) per ml of supernatant.

Detection of SstR

Cells were also stained with anti-SstR antibody and a fluorescent secondary antibody that reacts with the anti-SstR antibody. Cells were then observed microscopically.

### Results

Mutant MoMLV genes were designed in which the heterologous peptide sequence of human somatostatin (Sst) replaced part of the natural RBM of Env (MoMLV-Sst-RBM1, SEQ ID No 41-42), or the Sst sequence was inserted into the Proline-Rich Region (PRR; MoMLV-Sst-PRR; SEQ ID No 87) or the C-terminal end (MoMLV-Sst-230; SEQ ID No 88) of the N-terminal Domain (NTD) (FIG. 3A-B). Mutant MoMLV particles were produced that expressed the mutant env protein as their only Env.

The mutant MoMLV viral particles were incubated with 293 cells transfected with SstR, and the percentage of infected cells was quantitated. While recombinant viral particles containing wild-type MoMLV, Sst-PRR, or Sst-230 did not detectably infect the cells, the MoMLV-Sst-RBM1 viral particles infected 11.5+/−4.0% of the cells (FIG. 3C), approximately 57% of the fraction of SstR-expressing cells, (20.1+/−2.1%; FIG. 3D). Thus, replacement of the RBM with Sst conferred upon MoMLV env the ability to enter and infect cells that express SstR.

### Example 2

### Infection by MoMLV-Sst-RBM1 Recombinant Viral Particle is Mediated by Interaction with SstR on Target Cells

### Materials and Experimental Methods

Purified recombinant Sst-14 was obtained from Sigma-Aldrich. Inc.

### Results

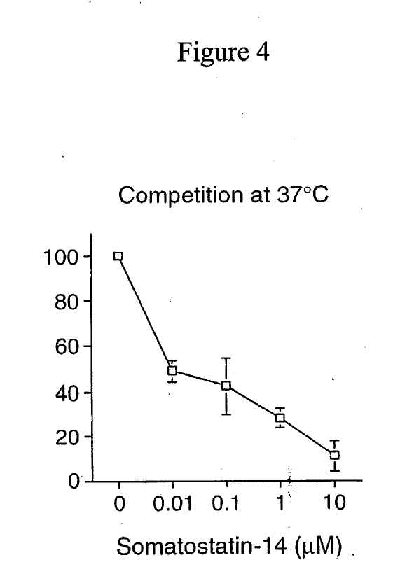

To test whether entry of the MoMLV-Sst-RBM1 recombinant viral particle into SstR-transfected 293 cells involved interaction with SstR, the transfected 293 cells were incubated with different concentrations of purified recombinant Sst-14 prior to addition of the recombinant viral particle. Pre-incubation of the cells with the recombinant Sst-14 facilitated blocking of the SstR on the surface of the cells prior to addition of the recombinant viral particle. Sst-14 inhibited infection of the cells in a dose-dependent manner (FIG. 4). Since infection requires viral entry, these results indicated that the MoMLV-Sst-RBM1 recombinant viral particle entered SstR-expressing 293 cells via interaction between the viral env protein and SstR.

### Example 3

### MoMLV-Sst-RBM1 is Unable to Enter Cells Through the Natural MoMLV Receptor

### Materials and Experimental Methods

3T3 Cells

Mouse NIH 3T3 cells were obtained from the ATCC.

Quantitation of Infection of NIH 3T3 Cells.

Infection of NIH 3T3 cells was measured in beta-gal-transducing units, which were quantified by end point dilution titration on the NIH 3T3 cells.

### Results

Mouse NIH 3T3 cells express the cationic amino acid transporter (ATRC-1; also delineated as CAT-1), the natural MoMLV receptor. The ability of wild-type MoMLV and the mutant MoMLV viruses to infect NIH 3T3 cells was tested. While wild-type MoMLV, Sst-PRR, and Sst-230, infected the cells, infection by the MoMLV-Sst-RBM1 mutant virus was decreased to undetectable levels, and increase of at least 5 orders of magnitude (FIG. 5). Sst-PRR infected roughly the same number of cells as wild-type MoMLV, and Sst-230 exhibited reduced, but measurable infection of the cells. Thus, replacement of the RBM with Sst conferred sharply reduced or abolished the ability of MoMLV to enter cells via the natural MoMLV receptor.

### Example 4

### MoMLV-Sst-RBM1 Viral Particles are Unable to Infect Human Neuroblastoma Cells Due to Cathepsin Activation

### Materials and Experimental Methods

Cells

SK-N-SH cells and NB 1643 cells were obtained from were obtained from Dr. Peter Houghton at St. Jude Children's Research Hospital, Memphis, Tenn. Sst-transfected cells were used as a positive control, as described in the above Examples.

### Results

In order to determine whether the MoMLV-Sst-RBM1 recombinant virus can enter neuroblastoma cells, the recombinant virus was incubated with 2 different Sst-expressing neuroblastoma cell lines, SK-N-SH and NB 1643. Neither neuroblastoma cell line was measurably infected (FIG. 6A), despite the presence of SstR.

The mutant MoMLV viruses may have been degraded in the lysosomes of the neuroblastoma cell lines by proteases such as cathepsins. In order to ascertain whether cathepsins played a role in protecting cells from MoMLV infection, NIH 3 T3 cells were incubated with wild-type MoMLV in the presence of different amounts of Cathepsin Inhibitor III, an inhibitor of cathepsins B, S, and L (CalBiochem-EMD Biosciences, San Diego, Calif.). The inhibitor increased infection of the cells in a dose-dependent manner, indicating that cathepsins played a role in protecting cells from MoMLV infection (FIG. 6B).

### Example 5

### Identification of an HBM and an RBM on MMTV env Via Sequence and Structural Alignment with Other Proteins

### Materials and Experimental Methods

Sequence Alignment

Segments within the putative MMTV RBM that were likely to have a β strand or α-helical structure were manually aligned with the known β and α helices of the Friend 57 strain of F-MLV RBM sequence (Davey, R A et al, J Virol. 71: 8096-8102, 1997; Davey, R A et al, J Virol. 73: 3758-3763, 1999; Jinno-Oue, A et al., J. Virol. 75: 12439-12445, 2001). The preliminary alignment was then submitted to Swiss-Model analysis for modeling of the MMTV structure. The WhatCheck and Tracelog reports from the Swiss-Model analysis identified residues likely to be misaligned, after which the F-MLV alignment was repeated with the misaligned residues moved one position amino- or carboxy-terminal. Additional adjustment of the alignment was made based on the results of the second set of models. Further adjustment of the position of residues several positions in either direction gave similar model coordinates.

### Results

In order to produce similar mutations in MMTV env protein to those generated in the MoMLV env protein, it was necessary to characterize the structure of MMTV env, including identification of the RBM. Consequently, alignment of the amino acid sequences of MMTV (C3H strain) and the Friend 57 strain of F-MLV was performed. First, the location of the receptor-binding motif (RBM) of MMTV env was delineated. In order to identify the RBM, a putative proline-rich region was first identified at residues 230 to 245 of the MMTV SU (FIG. 7A). This identification was facilitated by the location of the positively charged residue in position 46 (Arg), followed by hydrophobic amino acids (Leu-Val-Ala-Ala), a feature conserved in the beginning of the C-terminal domain of the murine gamma-retroviruses and bovine leukemia virus (BLV) SU. The sequence amino-terminal to the PRR was designated as the putative RBM of MMTV Env, by analogy to the known RBM of F-MLV. An RBM may also be referred to as a “Receptor Binding Domain (RBD).” The final alignment shown in FIG. 7A was used to generate the model shown in FIG. 7B, based on the structure of F-MLV env (Fass, D et al. Science 277: 1662-1666, 1997). Because of its location in the N-terminal portion of the protein, the RBD is in some cases referred to as the “N-terminal domain (NTD).”

The alignment shows the α helices and β strand regions of F-MLV, with the corresponding regions of MMTV that fold into similar structures, according to the SwissModel algorithms. Also shorn are the positions of the variable regions of F-MLV (VRA, VRB and VRC). In the three-dimensional model, MMTV contains similar regions (compare the left and center panels of FIG. 7B). The VRA, VRB and VRC regions of the F-MLV NTD are thought to be stabilized by thiol bonds. Although corresponding cysteine residues are not apparent in the linear alignment shown in FIG. 7A, there are two paired cysteine residues with the potential for disulfide bonding that lie at the base of VRA and VRC in the three dimensional model of MMTV env (dark and light short arrows in FIG. 7B). Similarly, although the putative N-linked glycosylation sites in MMTV do not align with F-MLV in the linear, alignment, they are found in regions similar to those in F-MLV in the model (long arrows).

A HBM has been found in env of both the Friend 57 strain (boxed sequence in FIG. 7A) and the PVC-211 variant of F-MLV. Analysis of the alignment revealed an HBM between residues 122 and 130 in MMTV SU (I₁₂₂KKKLPPKY₁₃₀) (FIG. 7A). The MMTV sequences were similar to defined mammalian consensus sequences (XBBXBX and XBBBXXBX, where X is any amino acid and B is a basic amino acid) (FIGS. 7A and C). On both the linear alignment (FIG. 7A) and the three-dimensional model, the MMTV HBM mapped to a region corresponding to that of the F-MLV HBM (compare region labeled “HBD” in left and center panels of FIG. 7B). Thus, linear- and three-dimensional alignment of MMTV env sequences with F-MLV env sequences identified an RBM and an HBM in MMTV env.

MMTV-env like sequences are found in primary human breast cancer tissue. To confirm the identification of the HBM based on the structural modeling of the C3H MMTV env, this sequence was compared with another isolate of MMTV and with the breast cancer sequences. The nearly canonical (standard) HBM in the MMTV env (I₁₂₂KKKLPPKY₁₃₀) was conserved among two isolates of wild-type MMTV (the RIII strain and the C3H strain), an MMTV virus adapted to the breast cancer cell line (the RIIIM strain), and two MMTV-like elements (h-MTVs) isolated from primary breast cancer samples (FIG. 7C). This finding confirmed the identification of an HBM in MMTV env.

### Example 6

### The HBM of MMTV env is Not Necessary for Virus Infection

### Materials & Experimental Methods

Cloning and Sequencing MMTV env Genes

Genomic DNA from the MCF-7/vp5 and MR/C1 cell lines was amplified by PCR using primers specific for MMTV env (P1, 5′-CTTGTGTTTTTCCACAGGATG (SEQ ID No 47); P2, 5′-TGCGAATTCCTATCGCTTGGCTCGAATTAAATC) (SEQ ID No 48) and directly sequenced. To clone the env genes, PCR primers were designed that included the same fragment of MMTV genomic proviral DNA present in pENV_{C3H}, the vector expressing the C3H env protein (as described in Dzuris, J L et al. Virol. 263: 418-426, 1999. The amplified fragments were cloned into pcDNA3.1 (Invitrogen. Inc.) to generate pENV_{RIII} (from MR/C1 cells) and pENV_{RIIIM} (from MCF-7/vp5 cells).

Site-Directed Mutagenesis

Plasmid pENV_{C3H} was the template for mutagenesis, using the QuickChange™ XL Site-Directed Mutagenesis Kit (Stratagene, Inc.). The Phe₄₀ codon was mutated to a Ser₄₀, Tyr₄₀ or Ala₄₀, and the Gly₄₂ codon to Glu₄₂ in 4 separate operations. To generate the HBM_{K-A} mutation in the pENV_{C3H}, the Lys₁₂₃, Lys₁₂₄, and Lys₁₂₅ codons, were mutated to Ala.

Cell Lines

293T human kidney epithelial cells were grown in Dulbecco minimal essential medium (DMEM)+10% fetal bovine serum (FBS). Normal mouse mammary gland (NmuMG) epithelial cells were grown in DMEM+10% FBS and 10 mg/ml insulin. The MCF-7/vp5 (obtained from A. Vaidya; derived by adapting MMTV(RIII) on MCF-7 human breast cancer cells) and MR/C1 (MMTV_{RIII}-infected mink lung) cell lines were grown in DMEM+10% FBS, 10 mg/ml insulin and 1 mM sodium pyruvate.

Pseudotyped Virus Preparation

MMTV Env-pseudotyped MoMLV viruses were made by transient co-transfection of 293T cells with pHit111 (comprising MoMLV genome and β-galactosidase marker), pHit60 (expressing MoMLV gag/pol genes) (Soneoka, Y et al, Nucl. Acids Res. 23: 628-633, 1995) and the pENV-based plasmids as described (Golovkina, T V et al. J. Virol. 72: 3066-3071, 1998). 2×10⁵ NMuMG cells were incubated with diluted pseudovirus supernatants containing polybrene (8 mg/ml) at 37° C. for two hours.

Antibody blocking studies were performed by pre-incubating pseudovirus for 10 minutes (min) prior to addition to cells with polyclonal goat anti-MMTV antisera diluted 1:2000 or hybridoma cell supernatants diluted 1:5. Heparan sulfate competition studies were performed by pre-incubating viruses with the indicated amounts of heparan sulfate (ICN Biochemicals; #97040) at 37° C. for one hour, then adding 8 mg/ml Polybrene and adding the mixture to infect NMuMG cells. After incubation for one hour, cells were washed and fresh media added.

### Results

To determine whether mutations in the HBM identified in the present invention had an effect on infection efficiency, pseudotyped viruses were generated containing mutations in the HBM. FIG. 8A shows that the relative infectivity of the HBM_{K-A} pseudotyped virus was decreased to about 20% of the wild type virus, showing that these three lysine residues in the HBM were important for infectious titer. The loss of infection was not due to a reduction in the levels of Env protein expression or incorporation into particles, as evidenced by Western blotting of transfected cell Sensates and purified pseudovirus (FIG. 8B).

To confirm that HBD_{K-A} mutation reduced MMTV infectivity through the loss of interaction with this proteoglycan, soluble heparan sulfate was added to the pseudovirus prior to infection. Treatment of wild-type virus with soluble heparan sulfate for one hour at 37° C. resulted in a dose-dependent decrease in infectious titer (FIG. 8C). The wild type virus titer at saturating levels of heparan sulfate was similar to the untreated HBD_{K-A} pseudovirus. In contrast, treatment of the DHBM pseudovirus with soluble heparan sulfate caused a minor decrease in the infectious titer, which was not further decreased with additional heparan sulfate. These findings showed that the HBM mediated MMTV infection by binding heparan sulfate.

### Example 7

### Comparison of Different MMTV env Sequences Confirms the Identification of the MMTV env Protein RBM

The env sequences from two isolates of wild-type MMTV (the RIII strain and the C3H strain) were compared to the other env sequences described in Example 7 to identify sequence variations that affected receptor binding. Most of the polymorphisms in the h-MMTV and RIIIM sequences were not unique to these viruses, but instead are found in other strains of MMTV. Moreover, none of the polymorphisms were found in all the human cell-associated viruses. However, 2 single-nucleotide alterations that resulted in non-conservative amino acid changes were found in the RBM identified in Example 5 (FIG. 7C). One alteration (Phe₄₀ to Ser₄₀) was identified in the sequence from one human breast cancer sample (h-MMTV1) but not the other (h-MMTV2). The second polymorphism (Gly₄₂ to Glu₄₂) was found in the env sequence from the RIIIM strain, adapted to MCF-7 cells, but not the parental virus. Only one other polymorphism was found in the RIIIM virus close to the C terminus of env (a semi-conservative Asp to Asn change).

Phe₄₀ and Gly₄₂ are in a five amino acid stretch of polar and hydrophobic residues directly adjacent to a glycosylation site, features often found at sites of protein-protein interaction in soluble proteins. The three-dimensional model of MMTV env (FIG. 7B) revealed that Ser₄₀ and Glu₄₂ were located on the outer surface of the molecule and formed a concave surface, consistent with a role in receptor interaction (circled, space-filled atoms). Thus, these findings confirmed identification the MMTV env protein RBM.

### Example 8

### The RBM of MMTV env is Necessary for Infectivity of MMTV

To determine the importance in infectivity of the RBM identified in the present invention, the Ser₄₀ and Glu₄₂ polymorphisms identified above were introduced into pENV_{C3H}, the wild-type MMTV Env construct used for producing pseudoviruses. Phe₄₀ was also changed to Ala₄₀ (nonconservative) and Tyr₄₀ (conservative).

To demonstrate that the mutant Env proteins were efficiently expressed, proteolytically processed, and incorporated into virions, total cell extracts from wild-type and mutant pENV_{C3H}-transfected 293T cells and purified pseudoviruses were analyzed by Western blot for the presence of env. The mutant envelope proteins were processed into mature SU and TM and stably integrated into virions to the same extent as wild-type Env (FIG. 9, top panel).

The pseudoviruses comprising the wild-type and variant envelope proteins were next tested for their ability to infect NMuMG cells. The Ser₄₀ and Ala₄₀ mutations completely abolished infectivity, the Glu₄₂ mutation did not significantly affect infection, and the conservative Tyr₄₀ mutant modestly decreased infection levels (FIG. 8, bottom panel). These findings confirmed the identification of the RBM and demonstrated that the RBM was necessary for infection.

### Example 9

### The RBM of MMTV env is Necessary for MMTV Binding to Mouse Cells

### Materials & Experimental Methods

Virus Binding Assay

100 ml of transfected cell supernatants were centrifuged at 25,000 rpm for 2 hours, and virus pellets were resuspended in 1.5 ml phosphate-buffered saline (PBS), pH 7.4+2% FBS and 1 mM EDTA. 0.5 ml of concentrated virus stock containing a fixed number of virus particles, as determined by Western blot analysis, were incubated with 2.5×10⁵ NMuMG cells in the presence of 8 mg/ml polybrene, 4° C., 1 hour. For heparan sulfate binding inhibition, the virus preparations were pre-incubated with 100 mg/ml heparan sulfate, 30 min, 37° C. Cells were washed, resuspended in 100 ml PBS+1% FBS, then incubated with 100 ml of goat anti-MMTV antisera, 1:100 dilution, 4° C., 30 min. Cells were washed and incubated with 100 ml FITC-conjugated rabbit anti-goat antibody, then washed and resuspended in 2% paraformaldehyde and subjected to FACS analysis. Data were acquired on a FACScan cytometer (Becton Dickinson, Mountainview, Calif.) and analyzed using CellQuest software (Becton Dickinson Immunocytometry Systems).

### Results

To investigate the role of the RBM identified in the present invention in virus binding to cells, virus-cell binding assays were performed. NMuMG cells were incubated with equal amounts of wild type or Ser₄₀ pseudovirus, then stained with anti-MMTV antibodies and analyzed by FACS to detect bound virus When wild-type virus was used (FIG. 10A), two distinct populations of cells bound high levels (mean channel fluorescence [MCF]=66.1; arrow 3 in FIG. 10), and low levels (MCF=9.8; arrow 1) of virus. Cells stained with anti-MMTV antibodies in the absence of virus had an MCF of 6.2 (arrow NV), demonstrating specificity of staining. In contrast, cells bound low, but not high levels of the Ser₄₀ pseudovirus (MCF=18.7: arrow 2).

To determine whether either of the populations (low and high virus-binding) resulted from non-receptor-mediated binding (i.e. through proteoglycan interactions with the HBD in the MMTV Env), binding assays were performed in the presence of 100 mg/ml heparan sulfate (FIG. 10B). While the high-binding population was still seen with the wild type pseudovirus (MCF=78.9), the number of cells in the population was diminished by about 2.5-fold. In contrast, fluorescence intensity of the low-virus binding populations of both wild type and Ser40 viruses were reduced to background levels. These results indicated that low-level binding was due to proteoglycan interactions, while high-level binding was due to specific receptor-binding interactions, further confirming the identification of the RBM of MMTV env and showing that the RBM was necessary for virus binding to cells.

### Example 10

### The RBM of MMTV env is Necessary for MMTV Binding to Mouse TfR1

### Materials & Experimental Methods

Generation of TRH3 Cells

TRH3 cells (a clonal isolate of 293T cells stabley expressing mouse TfR1) were generated by co-transfecting 293T cells with plasmids expressing TfR (Ross, S R et al, Proc. Natl. Acad. Sci. USA 99: 12386-12390, 2002) and pSV2neo (Esnault C et al Nucl Acids Res, 30: 11, 2002) comprising the neomycin resistance gene, followed by selection in G418 (100 mg/ml) and fluorescence activated cell sorting (FACS) for TfR expression using FACStar Plus (Becton Dickinson, Inc.) to select clonal isolates.

TRF1 Blocking Assay

TRH3 cells were incubated with rat anti-mTfRF1 antibody and et or Ser₄₀ MMTV_{C3H} under conditions of 1,200×g, room temperature, for 2 hours, in some cases pre-treating with goat anti-MMTV antiserum, (1 mg/ml MOPC-315; RDI, Flanders, N.J.) as described (Ross, S R, et al, Proc. Natl. Acad. Sci. USA 99: 12386-12390, 2002). Cells were washed with PBS+1% FBS, then stained with PE-conjugated rat-anti-mouse CD71 (TfR1) (PharMingen). 293T cells, which do not express TfR, were used as a specificity control. Data were acquired using a FACScan cytometer (Becton Dickinson) and analyzed using CELLQUEST software (Becton Dickinson Immunocytometry) after excluding dead cells by forward scatter/side scatter properties. P values were calculated using Student's T test: *—p≦:0.05; **—p≦0.005 (compared to TRH3 cells not bound to virus).

### Results

To determine whether the wild-type or Ser₄₀ pseudotyped virus interacted with TfR1, surface recognition of TfR1 by a monoclonal antibody was assessed in the presence and absence of the viruses (FIG. 11). TRH3 cells, which stably express TfR1, were incubated with pseudovirus at room temperature for 2 hours, then shifted to 4° C. and washed with azide-containing buffer to prevent internalization of TfR1. Surface recognition of TfR1 was then assessed by FACS analysis. Wild type, but not Ser₄₀, virus significantly decreased recognition of TfR1 (FIG. 11). Incubation of the wild type virus with anti-MMTV antisera blocked this effect, showing its dependence upon env protein. As a positive control for down-regulation of TfR1, TRH3 cells were incubated with the rat C2 monoclonal antibody (Ross, S R et al, Proc. Natl. Acad. Sci. USA 99: 12386-12390, 2002). These findings demonstrated that binding of MMTV to cells via the RBM identified in the present invention was mediated at least in part by interaction with TfR1.

### Example 11

### Monoclonal Antibodies that Block Infection Recognize the RBM

### Materials & Experimental Methods

Expression and Purification of RBM-GST Fusion Protein

A fragment encoding the RBM of the MMTV_{C3H} Env (amino acids 35 to 48; boxed sequence in FIG. 7C) was cloned into the BamHI site of pGEX-2T (Promega Biotech. Inc.) Protein was isolated from JTM109 bacteria transformed with MMTV_{C3H} Env-pGEX-2T by affinity purification with glutathione agarose beads (Pharmacia, Inc.) according to the manufacturer's instructions.

Western Blotting

Equal volumes of pseudovirus supernatants were resolved on 10% denaturing polyacrylamide gels. Proteins were transferred to nitrocellulose membrane and probed with goat anti-MMTV (1:3000 dilution), mouse anti-SU hybridoma supernatants (1:5 dilution) or rabbit anti-GST (1:3,000 dilution) and secondary antibodies, followed by detection with by Enhanced Chemiluminescence (ECL) kit (Amersham Biosciences, Inc.). Polyclonal rabbit anti-Glutathione S-transferase (GST) antibody was obtained from Sigma, Inc. (St. Louis, Mo.).

### Results

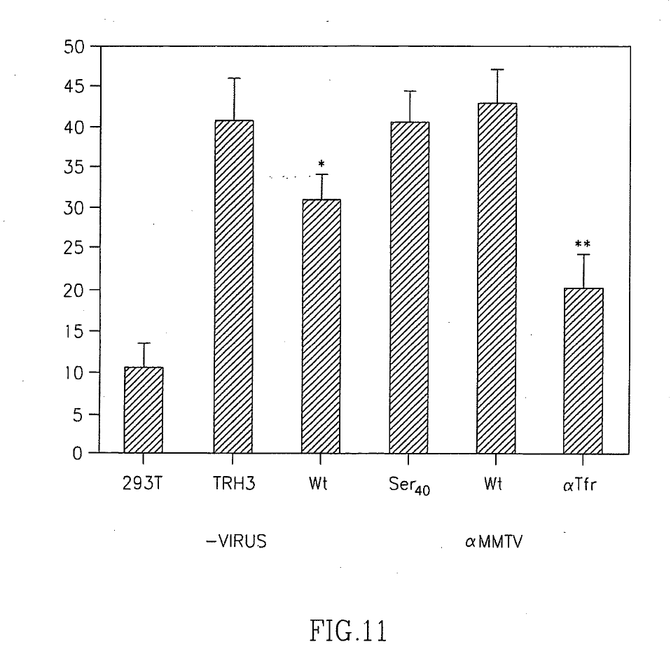

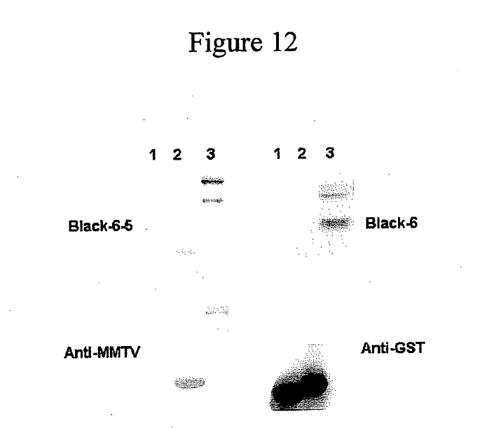

Four anti-Env monoclonal antibodies were tested for their ability to block infection and recognize the RBM identified in the present invention. Two of the antibodies, Black 6-5D and Black 8-6, blocked infection, while the other two, Black 6 and 2F10, were unable to block infection although they recognized MMTV Env by Western blot analysis (FIG. 12).

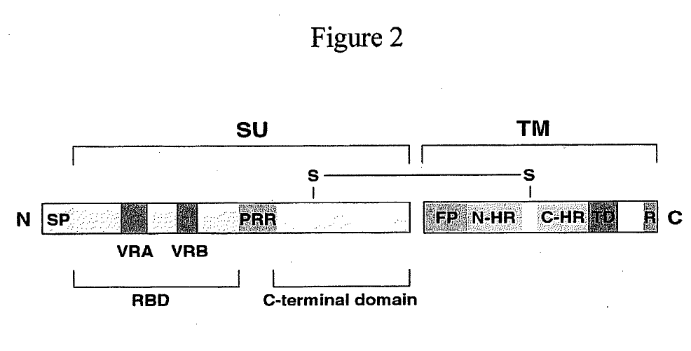

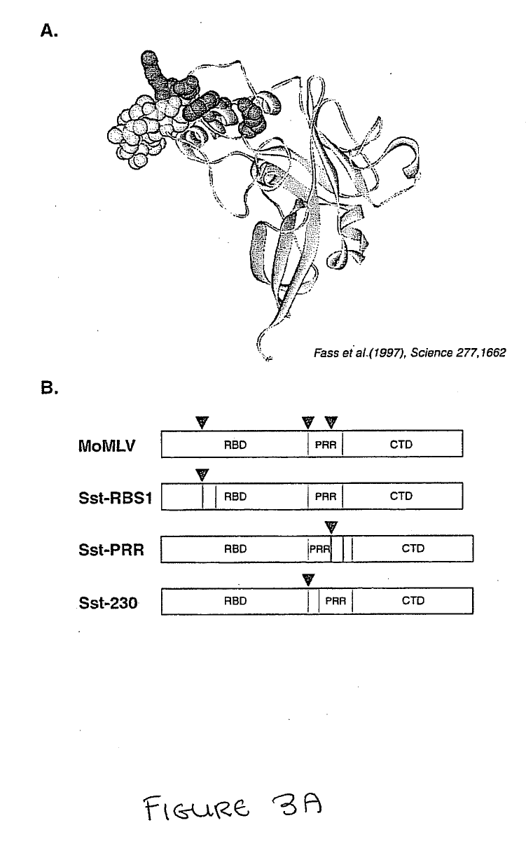

To determine if the antibodies bound the RBM, they were used to probe an RBM-GST fusion protein in Western blot analysis. Both Black 6-5D (FIG. 12B) and Black 8-6 (not shown) neutralizing monoclonal antibodies specifically bound RBM-GST but not GST alone; while the Black 6 (FIG. 12B) and 2F10 (not shown) antibodies did not bind RBM-GST. These findings demonstrate that the RBM of MMTV env identified in the present invention directly participates in receptor binding.

### Example 13

### Pseudotyping of MMTV env with the MoMLV env Cytoplasmic Tail Increases Infectivity

### Materials & Experimental Methods

Construction of MMTV-MoMLV Tail Chimera

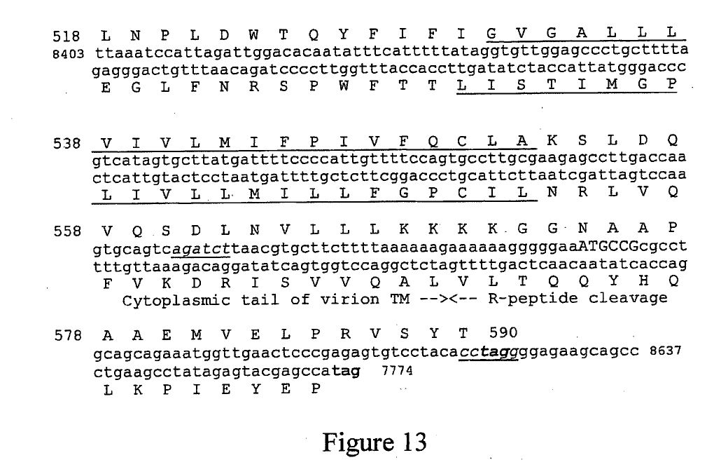

To create the MMTV-MoMLV tail chimera, the MMTV env gene was digested with Bgl II and AvrII (FIG. 13), and the corresponding sequence from MoMLV env was ligated into the plasmid.

### Results

The MMTV-Sst-RBM env protein described above was further modified by replacing (pseudotyping) its cytoplasmic tail with the corresponding portion of the MoMLV cytoplasmic tail, and the infectivity of the tail chimera was compared to MMTV-Sst-RBM env. The tail chimera exhibited a significant increase in infectivity relative to MMTV-Sst-RBM env.

These finding show that pseudotyping the cytoplasmic tail of env proteins, e.g. recombinant env proteins of the present invention, represents a method for increasing their infectivity.

### Example 14

### Point Mutation of Arg 95 of MoMLV-Sst-RBM1 Enhances Viral Infectivity

### Materials & Experimental Methods

Oligonucleotide-directed mutagenesis (QuikChange® kit) was used to construct variations in the nucleotide sequence encoding the MoMLV-Sst-RBM1. To produce R95D, the codon for R95 was mutated to encode aspartate. To produce W100A, the codon for W100 was mutated to encode alanine. Infectivity was assayed as described in Example 1.

### Results

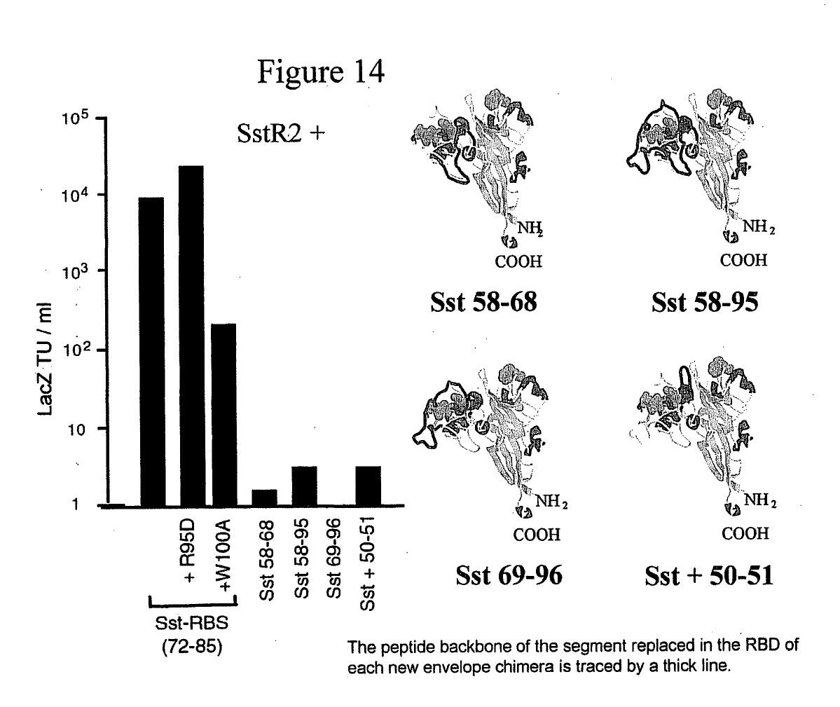

The effect of several mutations on the ability of MoMLV-Sst-RBM1 to infect SstR-transfected cells was determined. Modification of MoMLV-Sst-RBM1 with the R95D mutation increased infection by a two-fold margin, while modification with the W100A mutation slightly decreased infectivity (FIG. 14).

Thus, mutation of Arg 95 to Asp improves infectivity of MoMLV vectors containing a heterologous peptide.

### Example 15

### Replacement of Different Stretches of MoMLV-env by Sst Sequence also Confers Infectivity of SstR-Expressing Cells

### Materials & Experimental Methods

Additional mutagenesis was used to construct variants of MoMLV-Sst in which the Sst sequence was present in a different location. To produce MoMLV-Sst 58-68, MoMLV-Sst 58-95, MoMLV-Sst-69-96, and MoMLV-Sst 50-51, the codons encoding the indicated amino acids were replaced codons encoding Sst-14. Infectivity was assayed as described in Example 1.

### Results

The next experiment tested the ability to infect SstR-transfected cells of variants of MoMLV-Sst, in which the Sst sequence was present in a different location than MoMLV-Sst-RBM1. MoMLV-Sst-RBM1 exhibited the greatest efficiency of infection by a factor of several hundred-fold (FIG. 14).

Thus, a heterologous peptide need not exactly replace the RBM of MoMLV env protein in order to confer infectivity of cells containing surface proteins that interact with the peptide; rather, it can be present near the RBM in the 3-dimensional structure of the protein. However, exact- or near-exact replacement of the RBM with the heterologous sequence confers the highest infectivity of the variants tested.

### Example 16

### Env Proteins of Viruses Ordinarily Internalized by Clathrin-Independent Endocytosis Mediate Internalization by Interaction with Cellular Proteins that are Internalized via Clathrin-Dependent Endocytosis, and Vice-Versa

### Materials & Experimental Methods

Stable Transfection of HEK 293 Cells

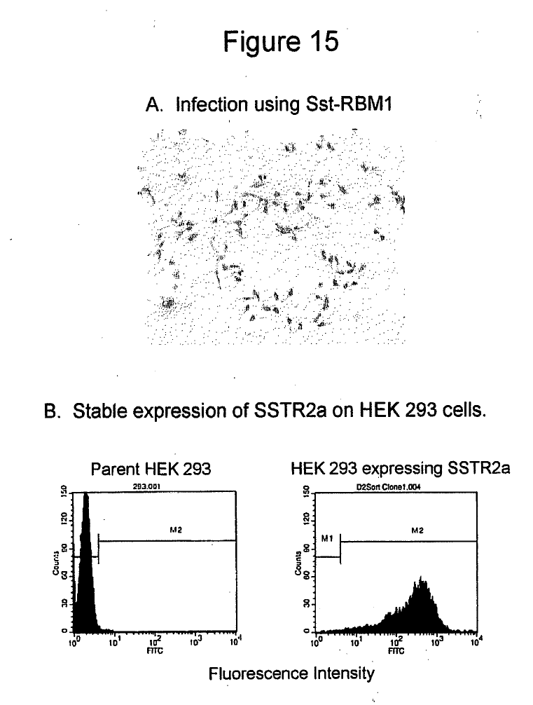

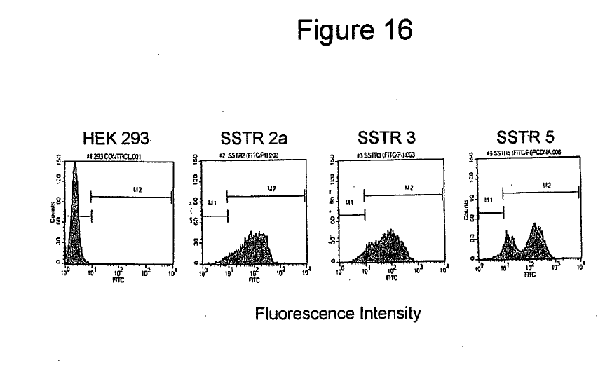

Mammalian expression plasmids containing cDNAs for human SSTR2a, SSTR3 and SSTR5 and the resistance gene for neomycin analog G418 were purchased from Affymetrix (California). These plasmids encode a nine amino acid HA epitope tag fused to the amino-terminus (corresponding to the extracellular domain) of each of the SSTR. Each cDNA was transfected into a separate population of HEK 293 cells (human embryonic kidney 293 cells) using standard CaPO₄ precipitation. Forty-eight hours later, cells were placed in growth medium containing 1 mg/ml G418 and maintained in this medium for four weeks to select for stable transfectants. Each population was then detached from culture plates, incubated with monoclonal mouse anti-HA antibody, then fluorescein-conjugated anti-mouse antibody, and sorted by FACS (fluorescence activated cell sorting; FIGS. 15-16) for cells exhibiting high levels of SSTR expression. The high fluorescence cells were cultured for several weeks, after which the sorting was repeated. High fluorescence cells were cultured to establish three populations of cells, each of which stably expressed one of SSTR2(, SSTR3 or SSTR5.

Stocks of retroviral vectors pseudotyped with the chimeric Sst-RBS envelope proteins and carrying a virus genome (including β-gal) were diluted serially and exposed overnight to cells from each of the three populations. Infection was measured as described in Example 1.

### Results

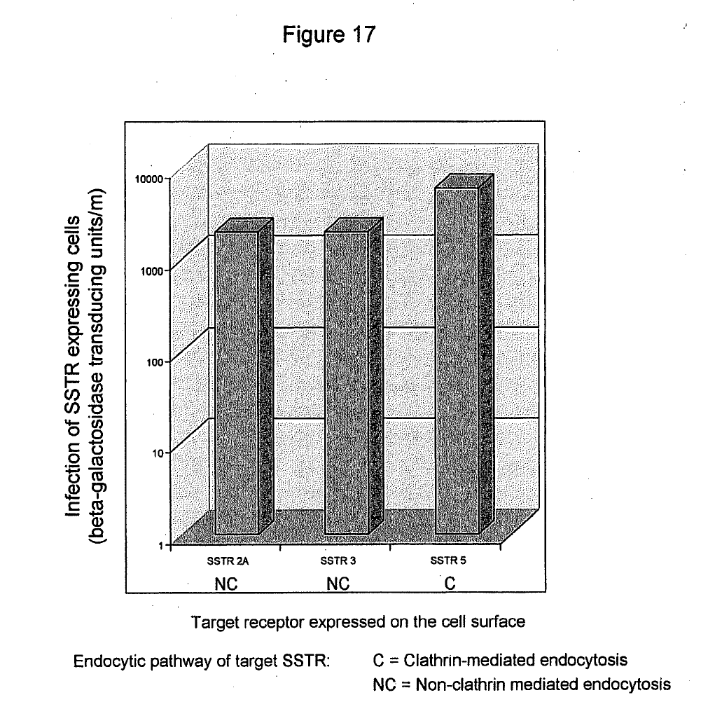

Sst-RBS envelope protein pseudotyped virus infected cell populations expressing not only SSTR2α, but also SSTR3 and SSTR5 (FIG. 17). Thus, although MoMLV env ordinarily mediates internalization by clathrin-independent endocytosis, recombinant env proteins derived from MoMLV env can enter cells using the clathrin mediated endocytic pathways at least as efficiently as clathrin-independent endocytic pathways.

These findings show that recombinant viral env proteins of the present invention can utilize cellular targets that are internalized via an endocytic uptake pathway different from the pathway utilized by the wild-type env protein from which the recombinant env protein was derived,

What is claimed is:

1. An isolated nucleic acid encoding for a receptor-binding motif of an MMTV env protein, said isolated nucleic acid having a nucleotide sequence selected from the sequences set forth in SEQ ID No 3-8, 17, 18, and 26.

2. A recombinant nucleic acid molecule comprising a heterologous nucleotide, said heterologous nucleotide corresponding to the isolated nucleic acid of claim 1.

3. A vector, cell, or packaging cell line comprising the recombinant nucleic acid molecule of claim 2.

4. An isolated nucleic acid encoding an MMTV env protein, said isolated nucleic acid comprising a mutation in a receptor-binding motif (RBM) of said env protein, said RBM having a nucleic acid sequence selected from the sequences set forth in SEQ ID No 3-8, 17, 18, and 26.

5. The isolated nucleic acid of claim 4, wherein said mutation comprises a replacement of all or part of said receptor-binding motif with a heterologous sequence encoding for a peptide that interacts with a cellular molecule.

6. The isolated nucleic acid of claim 4, further comprising a replacement of a sequence encoding a cytoplasmic tail of said MMTV env protein with a sequence encoding a cytoplasmic tail of a protein other than said MMTV env protein.

7. A vector, cell, or packaging cell line comprising the isolated nucleic acid of claim 5.

8. An isolated polypeptide encoded for by the isolated nucleic acid of claim 1.

9. An isolated polypeptide encoded for by the isolated nucleic acid of claim 2.

10. A vector, cell, or packaging cell line comprising the isolated polypeptide of claim 7.

11. An isolated polypeptide encoded for by the isolated nucleic acid of claim 4.

12. A recombinant viral particle, cell or packaging cell line comprising the isolated polypeptide of claim 10.

13. A recombinant viral particle, comprising
a. the isolated polypeptide of claim 11; and
b. a heterologous nucleic acid of interest.

14. A method for delivering a nucleic acid of interest or compound of interest to a target cell, comprising contacting said target cell with a recombinant viral particle or liposome comprising:
a. a nucleic acid of interest or compound of interest; and
b. a mutated retroviral or lentiviral env protein comprising a heterologous peptide;
whereby said heterologous peptide mediates uptake of said recombinant viral particle or liposome via a cellular target molecule, thereby delivering a nucleic acid of interest to a target cell.

15. The method of claim 14, wherein said mutated retroviral or lentiviral env protein is derived from a retrovirus or lentivirus resistant to lysosomal degradation.

16. The method of claim 14, whereby presence of said heterologous peptide diminishes or abrogates interaction of said retroviral or lentiviral env protein with a cellular molecule other than said cellular target molecule.

17. A method for enhancing an ability of a recombinant retroviral or lentiviral particle to infect a target cell, comprising contacting said target cell with an inhibitor of a lysosomal protease, whereby said inhibitor of a vacuolar enzyme prevents or impedes intracellular degradation of said recombinant retroviral or lentiviral particle, thereby enhancing delivery of a recombinant retroviral or lentiviral particle to a target cell.

18. An isolated nucleic acid encoding for a heparin-binding motif of an MMTV env protein, said isolated nucleic acid having a nucleotide sequence selected from the sequences set forth in SEQ ID No 27-32, 56-61, and 82.

19. A recombinant nucleic acid molecule comprising a heterologous nucleotide, said heterologous nucleotide corresponding to the isolated nucleic acid of claim 18.

20. A vector, cell, or packaging cell line comprising the recombinant nucleic acid molecule of claim 19.

21. An isolated polypeptide encoded bid the recombinant nucleic acid molecule of claim 19.

22. A vector, cell, or packaging cell line comprising the isolated polypeptide of claim 21.

23. An isolated nucleic acid encoding for a receptor-binding motif of an MoMLV env protein, said isolated nucleic acid having a nucleotide sequence selected from the sequences set forth in SEQ ID No 70-75.

24. A recombinant nucleic acid molecule comprising a heterologous nucleotide, said heterologous nucleotide corresponding to the isolated nucleic acid of claim 23.

25. A vector, cell, or packaging cell line comprising the recombinant nucleic acid molecule of claim 24.

26. An isolated nucleic acid encoding a mutated MoMLV env protein, said isolated nucleic acid comprising a mutation in a receptor-binding motif (RBM) of said env protein, said RBM having a nucleic acid sequence selected from the sequences set forth in SEQ ID No 70-75.

27. The isolated nucleic acid of claim 26, wherein said mutation comprises a replacement of all or part of said receptor-binding motif with a heterologous sequence encoding for a peptide that interacts with a cellular molecule.

28. A vector, cell, or packaging cell line comprising the isolated nucleic acid of claim 26.

29. An isolated polypeptide encoded for by the isolated nucleic acid of claim 26.

30. A vector, cell, or packaging cell line comprising the isolated polypeptide of claim 26.

31. A recombinant viral particle, comprising the isolated polypeptide of claim 29 and a heterologous nucleic acid of interest.

32. A method for delivering a nucleic acid of interest to a target cell, comprising contacting said target cell with a recombinant viral particle comprising a nucleic acid of interest and the isolated polypeptide of claim 29, whereby said isolated polypeptide mediates uptake of said recombinant viral particle via a cellular molecule, thereby delivering a nucleic acid of interest to a target cell.

33. A method for delivering a nucleic acid of interest or compound of interest to a tar-et cell via a clathrin-independent endocytosis, comprising contacting said target cell with a recombinant viral particle comprising:
a. a nucleic acid of interest or compound of interest; and
b. a mutated version of a wild-type env protein, wherein viruses containing said wild-type env protein are internalized via a clathrin-dependent endocytosis, and wherein said mutated version of a wild-type env protein comprises an insertion of a heterologous peptide that binds a cellular surface protein that capable of being internalized via a clathrin-independent endocytosis,
thereby delivering a nucleic acid of interest or compound of interest to a target cell via a clathrin-independent endocytosis.

34. A method for delivering a nucleic acid of interest or compound of interest to a target cell via a clathrin-dependent endocytosis, comprising contacting said target cell with a recombinant viral particle comprising:
a. a nucleic acid of interest or compound of interest; and
b. a mutated version of a wild-tape env protein, wherein viruses containing said wild-type env protein are internalized via a clathrin-independent endocytosis, and wherein said mutated version of a wild-type env protein comprises an insertion of a heterologous peptide that binds a cellular surface protein that capable of being internalized via a clathrin-dependent endocytosis,
thereby delivering a nucleic acid of interest or compound of interest to a target cell via a clathrin-dependent endocytosis.
